SEQUENCE LISTING (31 sequences)

SEQ ID NO: 1 - protein, 617 aa, Artificial Sequence
  li-HAEP amino acid sequence
MDDQRDLISNHEQLPILGNRPREPERCSRGALYTGVSVLVALLLAGQATTAYFLYQQQGRLDKLTITSQNLQLESLRMKLPKSAKPVSQMRMATPLLMRPMSMDNMLLGPVKNVTKYGNMTQDHVMHLLTRSGPLEYPQLKGTFPENLKHLKNSMDGVNWKIFESWMKQWLLFEMSKNSLEEKKPTEAPPKEPLDMEDLSSGLGVTRQELGQVTLGGGDSQIMYNYPAMLGHAGDMAGYAGTLQSLGAEIAVEQAALQSAWQGDTGITYQAWQAQWNQAMEDLVRAYHAMSSTHEANTMAMMARDTAEAAKWGGNTEQQWNFAGIEAAASAIQGNVTSIHSLLDEGKQSLTKLAAAWGGSGSEAYQGVQQKWDATATELNNALQNLARTISEAGQAMASTEGNVTGMFANNGGTENLTVQPERLGVLASHHDNAAVDASSGVEAAAGLGESVAITHGPYCSQFNDTLNVYLTAHNALGSSLHTAGVDLAKSLRIAAKIYSEADEAWRKAIDGLFTNNDDSFVTTQPEALAAAAANLQGIGTTMNAQNAAAAAPTTGVVPAAADEVSALTAAQFAAHAQMYQTVSAQAAAIHEMFVNTLVASSGSYAATEAANAAAAG

SEQ ID NO: 2 - DNA, 1854 bp, Artificial Sequence
  li-HAEP DNA sequence
atggacgatcagcgggacctgatctccaatcacgagcagctgccaatcctgggaaacaggcctagagagccagagaggtgctctcgcggcgccctgtacaccggcgtgagcgtgctggtggccctgctgctggcaggacaggccaccacagcctacttcctgtatcagcagcagggcaggctggataagctgaccatcacatcccagaatctgcagctggagtctctgcgcatgaagctgccaaagtccgccaagcccgtgtctcagatgaggatggccacccctctgctgatgcgcccaatgtctatggacaacatgctgctgggccccgtgaagaacgtgaccaagtacggcaatatgacacaggatcacgtgatgcacctgctgaccaggagcggaccactggagtatcctcagctgaagggcacatttcctgagaacctgaagcacctgaagaattctatggacggcgtgaactggaagatcttcgagagctggatgaagcagtggctgctgtttgagatgagcaagaattccctggaggagaagaagccaaccgaggcaccacctaaggagccactggacatggaggatctgagctccggcctgggcgtgaccaggcaggagctgggacaggtgacactgggaggaggcgacagccagatcatgtacaactatcctgccatgctgggacacgcaggcgatatggcaggatacgcaggcacactgcagagcctgggagcagagatcgcagtggagcaggccgccctgcagtccgcctggcagggcgacaccggaatcacataccaggcctggcaggcccagtggaatcaggccatggaggatctggtgcgggcctatcacgccatgtctagcacccacgaggccaacacaatggccatgatggccagagataccgcagaggcagcaaagtggggaggaaatacagagcagcagtggaacttcgcaggaatcgaggcagcagcaagcgccatccagggcaatgtgacctctatccacagcctgctggacgagggcaagcagtccctgacaaagctggcagcagcatggggaggctctggcagcgaggcatatcagggcgtgcagcagaagtgggatgccaccgccacagagctgaacaatgccctgcagaacctggcaaggaccatcagcgaggcaggacaggcaatggcatccaccgagggcaatgtgacaggcatgttcgccaacaatggcggcaccgagaacctgacagtgcagcctgagcgcctgggcgtgctggcatcccaccacgacaatgccgccgtggatgcctcctctggagtggaggcagcagcaggactgggagagagcgtggccatcacccacggcccatactgctcccagtttaacgacaccctgaacgtgtacctgacagcacacaacgcactgggcagctccctgcacacagcaggagtggatctggcaaagtccctgaggatcgccgccaagatctactctgaggccgacgaggcctggagaaaggccatcgatggcctgttcaccaacaatgacgatagctttgtgaccacacagccagaggccctggcagcagcagcagcaaacctgcagggcatcggcaccacaatgaacgcccagaatgcagcagcagcagcaccaaccacaggagtggtgcctgccgccgccgacgaggtgagcgccctgaccgccgcccagttcgccgcccacgcccagatgtaccagacagtgtctgcccaggcagcagcaatccacgagatgtttgtgaataccctggtggcctctagcggctcctatgcagcaacagaggcagcaaacgcagcagcagccggctga

SEQ ID NO: 3 - protein, 636 aa, Artificial Sequence
  li-HAEPA amino acid sequence
MDDQRDLISNHEQLPILGNRPREPERCSRGALYTGVSVLVALLLAGQATTAYFLYQQQGRLDKLTITSQNLQLESLRMKLPKSAKPVSQMRMATPLLMRPMSMDNMLLGPVKNVTKYGNMTQDHVMHLLTRSGPLEYPQLKGTFPENLKHLKNSMDGVNWKIFESWMKQWLLFEMSKNSLEEKKPTEAPPKEPLDMEDLSSGLGVTRQELGQVTLGGGDSQIMYNYPAMLGHAGDMAGYAGTLQSLGAEIAVEQAALQSAWQGDTGITYQAWQAQWNQAMEDLVRAYHAMSSTHEANTMAMMARDTAEAAKWGGNTEQQWNFAGIEAAASAIQGNVTSIHSLLDEGKQSLTKLAAAWGGSGSEAYQGVQQKWDATATELNNALQNLARTISEAGQAMASTEGNVTGMFANNGGTENLTVQPERLGVLASHHDNAAVDASSGVEAAAGLGESVAITHGPYCSQFNDTLNVYLTAHNALGSSLHTAGVDLAKSLRIAAKIYSEADEAWRKAIDGLFTNNDDSFVTTQPEALAAAAANLQGIGTTMNAQNAAAAAPTTGVVPAAADEVSALTAAQFAAHAQMYQTVSAQAAAIHEMFVNTLVASSGSYAATEAANAAAAQDAYNAGGGHNGVFDFPDSG

SEQ ID NO: 4 - DNA, 1911 bp, Artificial Sequence
  li-HAEPA DNA sequence
atggacgatcagcgggacctgatctccaatcacgagcagctgccaatcctgggaaacaggcctagagagccagagaggtgctctcgcggcgccctgtacaccggcgtgagcgtgctggtggccctgctgctggcaggacaggccaccacagcctacttcctgtatcagcagcagggcaggctggataagctgaccatcacatcccagaatctgcagctggagtctctgcgcatgaagctgccaaagtccgccaagcccgtgtctcagatgaggatggccacccctctgctgatgcgcccaatgtctatggacaacatgctgctgggccccgtgaagaacgtgaccaagtacggcaatatgacacaggatcacgtgatgcacctgctgaccaggagcggaccactggagtatcctcagctgaagggcacatttcctgagaacctgaagcacctgaagaattctatggacggcgtgaactggaagatcttcgagagctggatgaagcagtggctgctgtttgagatgagcaagaattccctggaggagaagaagccaaccgaggcaccacctaaggagccactggacatggaggatctgagctccggcctgggcgtgaccaggcaggagctgggacaggtgacactgggaggaggcgacagccagatcatgtacaactatcctgccatgctgggacacgcaggcgatatggcaggatacgcaggcacactgcagagcctgggagcagagatcgcagtggagcaggccgccctgcagtccgcctggcagggcgacaccggaatcacataccaggcctggcaggcccagtggaatcaggccatggaggatctggtgcgggcctatcacgccatgtctagcacccacgaggccaacacaatggccatgatggccagagataccgcagaggcagcaaagtggggaggaaatacagagcagcagtggaacttcgcaggaatcgaggcagcagcaagcgccatccagggcaatgtgacctctatccacagcctgctggacgagggcaagcagtccctgacaaagctggcagcagcatggggaggctctggcagcgaggcatatcagggcgtgcagcagaagtgggatgccaccgccacagagctgaacaatgccctgcagaacctggcaaggaccatcagcgaggcaggacaggcaatggcatccaccgagggcaatgtgacaggcatgttcgccaacaatggcggcaccgagaacctgacagtgcagcctgagcgcctgggcgtgctggcatcccaccacgacaatgccgccgtggatgcctcctctggagtggaggcagcagcaggactgggagagagcgtggccatcacccacggcccatactgctcccagtttaacgacaccctgaacgtgtacctgacagcacacaacgcactgggcagctccctgcacacagcaggagtggatctggcaaagtccctgaggatcgccgccaagatctactctgaggccgacgaggcctggagaaaggccatcgatggcctgttcaccaacaatgacgatagctttgtgaccacacagccagaggccctggcagcagcagcagcaaacctgcagggcatcggcaccacaatgaacgcccagaatgcagcagcagcagcaccaaccacaggagtggtgcctgccgccgccgacgaggtgagcgccctgaccgccgcccagttcgccgcccacgcccagatgtaccagacagtgtctgcccaggcagcagcaatccacgagatgtttgtgaataccctggtggcctctagcggctcctacgcagcaacagaggcagcaaacgcagcagcagcccaggacgcttacaacgcaggaggcggacacaacggcgtctttgactttcccgattccggctga

SEQ ID NO: 5 - protein, 315 aa, Artificial Sequence
  li-EsxH amino acid sequence
MDDQRDLISNHEQLPILGNRPREPERCSRGALYTGVSVLVALLLAGQATTAYFLYQQQGRLDKLTITSQNLQLESLRMKLPKSAKPVSQMRMATPLLMRPMSMDNMLLGPVKNVTKYGNMTQDHVMHLLTRSGPLEYPQLKGTFPENLKHLKNSMDGVNWKIFESWMKQWLLFEMSKNSLEEKKPTEAPPKEPLDMEDLSSGLGVTRQELGQVTLGAGAMSQIMYNYPAMLGHAGDMAGYAGTLQSLGAEIAVEQAALQSAWQGDTGITYQAWQAQWNQAMEDLVRAYHAMSSTHEANTMAMMARDTAEAAKWGG

SEQ ID NO: 6 - DNA, 948 bp, Artificial Sequence
  li-EsxH DNA sequence
atggatgaccaacgcgacctcatctctaaccatgagcaattgcccatactgggcaaccgccctagagagccagaaaggtgcagccgtggagctctgtacaccggtgtttctgtcctggtggctctgctcttggctgggcaggccactactgcttacttcctgtaccagcaacagggccgcctagacaagctgaccatcacctcccagaacctgcaactggagagccttcgcatgaagcttccgaaatctgccaaacctgtgagccagatgcggatggctactcccttgctgatgcgtccaatgtccatggataacatgctccttgggcctgtgaagaacgttaccaagtacggcaacatgacccaggaccatgtgatgcatctgctcacgaggtctggacccctggagtacccgcagctgaaggggaccttcccagagaatctgaagcatcttaagaactccatggatggcgtgaactggaagatcttcgagagctggatgaagcagtggctcttgtttgagatgagcaagaactccctggaggagaagaagcccacagaggctccacctaaagagccactggacatggaagacctatcttctggcctgggagtgaccaggcaggaactgggtcaagtcaccctgggagctggagctatgtcccagattatgtacaactatccagcaatgttggggcatgccggggatatggccggctatgcaggcacccttcaatccctgggagccgaaattgccgtagagcaggctgcccttcagagtgcatggcaaggcgatactggtatcacataccaagcgtggcaggcacagtggaatcaggcaatggaagatttggtgcgagcttatcatgccatgagttccacacacgaagccaacaccatggcgatgatggctagggataccgccgaagctgccaagtggggaggatga

SEQ ID NO: 7 - protein, 218 aa, Artificial Sequence
  TfR1-118-EsxH amino acid sequence
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAGAGAMSQIMYNYPAMLGHAGDMAGYAGTLQSLGAEIAVEQAALQSAWQGDTGITYQAWQAQWNQAMEDLVRAYHAMSSTHEANTMAMMARDTAEAAKWGG

SEQ ID NO: 8 - DNA, 657 bp, Artificial Sequence
  TfR1-118-EsxH DNA sequence
atgatggatcaagctagatcagcattctctaacttgtttggtggagaaccattgtcatatacccggttcagcctggctcggcaagtagatggcgataacagtcatgtggagatgaaacttgctgtagatgaagaagaaaatgctgacaataacacaaaggccaatgtcacaaaaccaaaaaggtgtagtggaagtatctgctatgggactattgctgtgatcgtctttttcttgattggatttatgattggctacttgggctattgtaaaggggtagaaccaaaaactgagtgtgagagactggcaggaaccgagtctccagtgagggaggagccaggagaggacttccctgcaggagctggagctatgtcccagattatgtacaactatccagcaatgttggggcatgccggggatatggccggctatgcaggcacccttcaatccctgggagccgaaattgccgtagagcaggctgcccttcagagtgcatggcaaggcgatactggtatcacataccaagcgtggcaggcacagtggaatcaggcaatggaagatttggtgcgagcttatcatgccatgagttccacacacgaagccaacaccatggcgatgatggctagggataccgccgaagctgccaagtggggaggatga

SEQ ID NO: 9 - protein, 175 aa, Artificial Sequence
  SP-EsxH-MITD amino acid sequence
MARVTAPRTVLLLLSGALALTETWAMSQIMYNYPAMLGHAGDMAGYAGTLQSLGAEIAVEQAALQSAWQGDTGITYQAWQAQWNQAMEDLVRAYHAMSSTHEANTMAMMARDTAEAAKWGGVGIVAGLAVLAVVVIGAVVATVMCRRKSSGGKGGSYSQAASSDSAQGSDVSLTA

SEQ ID NO: 10 - DNA, 528 bp, Artificial Sequence
  SP-EsxH-MITD DNA sequence
atggctcgggtcacggcgccccgaaccgtcctcctgctgctctcgggagccctggccctgaccgagacctgggccatgtcccagattatgtacaactatccagcaatgttggggcatgccggggatatggccggctatgcaggcacccttcaatccctgggagccgaaattgccgtagagcaggctgcccttcagagtgcatggcaaggcgatactggtatcacataccaagcgtggcaggcacagtggaatcaggcaatggaagatttggtgcgagcttatcatgccatgagttccacacacgaagccaacaccatggcgatgatggctagggataccgccgaagctgccaagtggggaggagtgggcattgttgctggcctggctgtcctagcagttgtggtcatcggagctgtggtcgctactgtgatgtgtaggaggaagagctcaggtggaaaaggagggagctactctcaggctgcgtccagcgacagtgcccagggctctgatgtgtctctcacagcttga

SEQ ID NO: 11 - protein, 232 aa, Homo sapiens
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVPM

SEQ ID NO: 12 - DNA, 699 bp, Homo sapiens
atgcacaggaggagaagcaggagctgtcgggaagatcagaagccagtcatggatgaccagcgcgaccttatctccaacaatgagcaactgcccatgctgggccggcgccctggggccccggagagcaagtgcagccgcggagccctgtacacaggcttttccatcctggtgactctgctcctcgctggccaggccaccaccgcctacttcctgtaccagcagcagggccggctggacaaactgacagtcacctcccagaacctgcagctggagaacctgcgcatgaagcttcccaagcctcccaagcctgtgagcaagatgcgcatggccaccccgctgctgatgcaggcgctgcccatgggagccctgccccaggggcccatgcagaatgccaccaagtatggcaacatgacagaggaccatgtgatgcacctgctccagaatgctgaccccctgaaggtgtacccgccactgaaggggagcttcccggagaacctgagacaccttaagaacaccatggagaccatagactggaaggtctttgagagctggatgcaccattggctcctgtttgaaatgagcaggcactccttggagcaaaagcccactgacgctccaccgaaagagtcactggaactggaggacccgtcttctgggctgggtgtgaccaagcaggatctgggcccagtccccatgtga

SEQ ID NO: 13 - protein, 118 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPA

SEQ ID NO: 14 - DNA, 354 bp, Homo sapiens
atgatggatcaagctagatcagcattctctaacttgtttggtggagaaccattgtcatatacccggttcagcctggctcggcaagtagatggcgataacagtcatgtggagatgaaacttgctgtagatgaagaagaaaatgctgacaataacacaaaggccaatgtcacaaaaccaaaaaggtgtagtggaagtatctgctatgggactattgctgtgatcgtctttttcttgattggatttatgattggctacttgggctattgtaaaggggtagaaccaaaaactgagtgtgagagactggcaggaaccgagtctccagtgagggaggagccaggagaggacttccctgca

SEQ ID NO: 15 - protein, 9 aa, Mycobacterium tuberculosis
GYAGTLQSL

SEQ ID NO: 16 - protein, 15 aa, Mycobacterium tuberculosis
STHEANTMAMMARDT

SEQ ID NO: 17 - protein, 20 aa, Mycobacterium tuberculosis
MTEQQWNFAGIEAAASAIQG

SEQ ID NO: 18 - protein, 18 aa, Mycobacterium tuberculosis
MSFVTTQPEALAAAAANL

SEQ ID NO: 19 - protein, 20 aa, Mycobacterium tuberculosis
QDAYNAGGGHNGVFDFPDSG

SEQ ID NO: 20 - DNA, 7129 bp, Artificial Sequence
  pFlap-SP1b2m-GFP-WPREm
  misc_feature (3195)..(3200): BamHI
  misc_feature (3953)..(3958): XhoI
ggcgttgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcccgacattgattattgactagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccacggcgcgcccttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacggaaaccctgcagggaattccccagctgtagttataaacagaagttctccttctgctaggtagcattcaaagatcttaatcttctgggtttccgttttctcgaatgaaaaatgcaggtccgagcagttaactggcgggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgagcgatcggatccccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagctcgagaattcccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcggtacctttaagaccaatgacttacaaggcagctgtagatcttagccactttttaaaagaaaaggggggactggaagggctaattcactcccaacgaagacaaaatcgtcgagagatgctgcatataagcagctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagttctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcttgctgcttcgcgatgtacgggccagatatacgc

SEQ ID NO: 21 - DNA, 800 bp, Artificial Sequence
  SP1-beta2 microglobulin promoter
cggccgccagtgtgctggaattcgcccttggcgcgcccttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacggaaaccctgcagggaattccccagctgtagttataaacagaagttctccttctgctaggtagcattcaaagatcttaatcttctgggtttccgttttctcgaatgaaaaatgcaggtccgagcagttaactggcgggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgagcgatcggatccgccac

SEQ ID NO: 22 - DNA, 471 bp, Artificial Sequence
  BCUAG promoter
gatctgttaacttccccgaagtagcaatgtatttcccagaaaaggaataatccttctgggaattctggcgaggtttccgggaaagcagcaccgcccttggccgtcctgccaatttcactttctagtttcactttcccttttgtaactaaaatgtaaatgacataggaaaactgaaagggagaagtgaaagtgggaaattcctctgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgagtaacaactccgccccattgacgcaaatgggcggtaggcgtgtacggtgggaggtctatataa

SEQ ID NO: 23 - DNA, 605 bp, Woodchuck hepatitis virus
aattcccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggg

SEQ ID NO: 24 - protein, 332 aa, Artificial Sequence
  human li - EsxH
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVPMGAGAMSQIMYNYPAMLGHAGDMAGYAGTLQSLGAEIAVEQAALQSAWQGDTGITYQAWQAQWNQAMEDLVRAYHAMSSTHEANTMAMMARDTAEAAKWGG

SEQ ID NO: 25 - DNA, 7408 bp, Artificial Sequence
  pFlap-SP1b2m-hli-EsxH-WPREm
  misc_feature (87)..(164): SV40 ORI
  misc_feature (233)..(868): HIV1-5LTR
  misc_feature (870)..(1086): HIV-1 psi
  misc_feature (1533)..(1766): RRE
  misc_feature (2288)..(2411): cPPT-CTS
  misc_feature (2461)..(2691): SP1
  misc_feature (2693)..(3208): b2m-promotor
  misc_feature (3213)..(3218): BamHI
  misc_feature (3225)..(3920): Human li
  misc_feature (3933)..(4223): EsxH
  misc_feature (4224)..(4229): XhoI
  misc_feature (4230)..(4834): WPRE-mutated
  misc_feature (4904)..(5165): LTR truncated
  misc_feature (5202)..(5429): bGH PolyA
  misc_feature (5605)..(6396): Kan/neoR
  misc_feature (6700)..(7328): ColE1 origin
cgcgttgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcccgacattgattattgactagttggaagggctaattcactcccaacgaagacaagatatccttgatctgtggatctaccacacacaaggctacttccctgattagcagaactacacaccagggccagggatcagatatccactgacctttggatggtgctacaagctagtaccagttgagccagagaagttagaagaagccaacaaaggagagaacaccagcttgttacaacctgtgagcctgcatgggatggatgacccggagagagaagtgttagagtggaggtttgacagccgcctagcatttcatcacggtggcccgagagctgcatccggagtacttcaagaactgctgatatcgagcttgctacaagggactttccgctgggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggctgatacgcgtggagttcccttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacggaaaccctgcagggaattccccagctgtagttataaacagaagttctccttctgctaggtagcattcaaagatcttaatcttctgggtttccgttttctcgaatgaaaaatgcaggtccgagcagttaactggcgggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgagcgatcggatccgccaccatgcacaggaggagaagcaggagctgtcgggaagatcagaagccagtcatggatgaccagcgcgaccttatctccaacaatgagcaactgcccatgctgggccggcgccctggggccccggagagcaagtgcagccgcggagccctgtacacaggcttttccatcctggtgactctgctcctcgctggccaggccaccaccgcctacttcctgtaccagcagcagggccggctggacaaactgacagtcacctcccagaacctgcagctggagaacctgcgcatgaagcttcccaagcctcccaagcctgtgagcaagatgcgcatggccaccccgctgctgatgcaggcgctgcccatgggagccctgccccaggggcccatgcagaatgccaccaagtatggcaacatgacagaggaccatgtgatgcacctgctccagaatgctgaccccctgaaggtgtacccgccactgaaggggagcttcccggagaacctgagacaccttaagaacaccatggagaccatagactggaaggtctttgagagctggatgcaccattggctcctgtttgaaatgagcaggcactccttggagcaaaagcccactgacgctccaccgaaagagtcactggaactggaggacccgtcttctgggctgggtgtgaccaagcaggatctgggcccagtccccatgggagctggagctatgtcccagattatgtacaactatccagcaatgttggggcatgccggggatatggccggctatgcaggcacccttcaatccctgggagccgaaattgccgtagagcaggctgcccttcagagtgcatggcaaggcgatactggtatcacataccaagcgtggcaggcacagtggaatcaggcaatggaagatttggtgcgagcttatcatgccatgagttccacacacgaagccaacaccatggcgatgatggctagggataccgccgaagctgccaagtggggaggatgactcgagaattcccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggggtacctttaagaccaatgacttacaaggcagctgtagatcttagccactttttaaaagaaaaggggggactggaagggctaattcactcccaacgaagacaagatcgtcgagagatgctgcatataagcagctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagttctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttcttgactcttcgcgatgtacgggccagatatacgcg

SEQ ID NO: 26 - DNA, 7063 bp, Artificial Sequence
  pFlap-SP1b2m-hTfR-EsxH-WPREm
  misc_feature (87)..(164): SV40 ORI
  misc_feature (233)..(868): HIV1-5LTR
  misc_feature (870)..(1086): HIV-1 psi
  misc_feature (1533)..(1766): RRE
  misc_feature (2288)..(2411): cPPT-CTS
  misc_feature (2461)..(2691): SP1
  misc_feature (2693)..(3208): b2m-promotor
  misc_feature (3213)..(3218): BamHI
  misc_feature (3225)..(3575): hTfR
  misc_feature (3588)..(3878): EsxH
  misc_feature (3879)..(3884): XhoI
  misc_feature (3885)..(4489): WPRE-mutated
  misc_feature (4559)..(4820): LTR truncated
  misc_feature (4857)..(5084): bGH PolyA
  misc_feature (5260)..(6051): Kan/neoR
  misc_feature (6355)..(6983): ColE1 origin
cgcgttgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcccgacattgattattgactagttggaagggctaattcactcccaacgaagacaagatatccttgatctgtggatctaccacacacaaggctacttccctgattagcagaactacacaccagggccagggatcagatatccactgacctttggatggtgctacaagctagtaccagttgagccagagaagttagaagaagccaacaaaggagagaacaccagcttgttacaacctgtgagcctgcatgggatggatgacccggagagagaagtgttagagtggaggtttgacagccgcctagcatttcatcacggtggcccgagagctgcatccggagtacttcaagaactgctgatatcgagcttgctacaagggactttccgctgggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggctgatacgcgtggagttcccttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacggaaaccctgcagggaattccccagctgtagttataaacagaagttctccttctgctaggtagcattcaaagatcttaatcttctgggtttccgttttctcgaatgaaaaatgcaggtccgagcagttaactggcgggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgagcgatcggatccgccaccatggatcaagctagatcagcattctctaacttgtttggtggagaaccattgtcatatacccggttcagcctggctcggcaagtagatggcgataacagtcatgtggagatgaaacttgctgtagatgaagaagaaaatgctgacaataacacaaaggccaatgtcacaaaaccaaaaaggtgtagtggaagtatctgctatgggactattgctgtgatcgtctttttcttgattggatttatgattggctacttgggctattgtaaaggggtagaaccaaaaactgagtgtgagagactggcaggaaccgagtctccagtgagggaggagccaggagaggacttccctgcaggagctggagctatgtcccagattatgtacaactatccagcaatgttggggcatgccggggatatggccggctatgcaggcacccttcaatccctgggagccgaaattgccgtagagcaggctgcccttcagagtgcatggcaaggcgatactggtatcacataccaagcgtggcaggcacagtggaatcaggcaatggaagatttggtgcgagcttatcatgccatgagttccacacacgaagccaacaccatggcgatgatggctagggataccgccgaagctgccaagtggggaggatgactcgagaattcccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggggtacctttaagaccaatgacttacaaggcagctgtagatcttagccactttttaaaagaaaaggggggactggaagggctaattcactcccaacgaagacaagatcgtcgagagatgctgcatataagcagctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagttctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttcttgactcttcgcgatgtacgggccagatatacgcg

SEQ ID NO: 27 - protein, 4 aa, Artificial Sequence
  linker
GGGD

SEQ ID NO: 28 - protein, 4 aa, Artificial Sequence
  linker
NNGG

SEQ ID NO: 29 - protein, 4 aa, Artificial Sequence
  linker
NNDD

SEQ ID NO: 30 - DNA, 720 bp, Artificial Sequence
  Nucleotidic sequence of Green Fluorescent Protein (GFP) gene (codon optimized)
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 31 - protein, 239 aa, Artificial Sequence
  Amino-Acid sequence of Green Fluorescent Protein (GFP) gene
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

(19) United States
(12) Patent Application Publication
(10) Pub. No.: US 2025/0319173 A1
(43) Pub. Date: Oct. 16, 2025

(54) LENTIVIRAL VECTORS TARGETING ANTIGENS TO MHC-II PATHWAY AND INDUCING PROTECTIVE CD8+ AND CD4+ T-CELL IMMUNITY IN A HOST

(71) Applicants: INSTITUT PASTEUR, PARIS CEDEX 15 (FR); THERAVECTYS, PARIS CEDEX 15 (FR)

(72) Inventors: Pierre CHARNEAU, PARIS (FR); Laleh MAJLESSI, PARIS (FR); Jodie LOPEZ, PARIS (FR); Francois ANNA, PARIS (FR); Catherine BLANC, PARIS (FR); Fanny MONCOQ, PARIS (FR)

(21) Appl. No.: 18/549,286

(22) PCT Filed: Mar. 11, 2022
(86) PCT No.: PCT/EP2022/056390

§ 371 Date: Sep. 6, 2023

(30) Foreign Application Priority Data
Mar. 12, 2021 (EP) 21305317.6

(51) Int. Cl. A61K 39/04; A61K 38/00; A61K 39/00; A61K 39/385; A61P 31/06; C07K 14/705; C07K 14/74; C12N 5/071; C12N 5/09; C12N 15/86

(52) U.S. Cl. CPC A61K 39/04; A61K 39/385; A61P 31/06; C07K 14/70539; C07K 14/70582; C12N 5/0686; C12N 5/0694; C12N 15/86; A61K 38/00; A61K 2039/55561; A61K 2039/572; A61K 2039/575; A61K 2039/605; C07K 2319/03; C12N 2740/16022; C12N 2740/16034; C12N 2740/16043; C12N 2740/16052; C12N 2740/16222; C12N 2760/20222; C12N 2830/48

(57) ABSTRACT

A recombinant lentiviral vector genome comprising a polynucleotide encoding a fusion polypeptide, wherein said fusion polypeptide comprises, arranged from N-terminal to C-terminal ends: a first polypeptide comprising (i) an MHC-ll-associated light invariant chain (li), or (ii)) the transmembrane domain of the transferrin receptor (TfR) and at least one antigenic polypeptide of a pathogen. The invention also relates to a lentiviral vector and pharmaceutical compositions comprising it.

### FIELD OF THE INVENTION

The invention relates to lentiviral vectors designed to provide a new generation of vectors leveraged to route immunogens not only to MHC-I but also to MHC-II pathways, and to induce both CD4⁺ and CD8⁺ T-cell responses.

In particular, the invention relates to such lentiviral vectors expressing antigen(s) selected for their interest in eliciting an immunological response in a host, in particular a mammalian host, especially a human host in need thereof wherein the immunological response encompasses a CD4+ T-cell response. The antigens may be expressed from an insert in the lentiviral backbone of the vector consisting of a polynucleotide encoding a fusion polypeptide comprising an MHC-II pathway-addressing molecule fused with a single antigen or multiple antigens.

The lentiviral vector of the invention is provided for use in the design of immunological compositions, preferably of a vaccine candidate, in particular a vaccine suitable for a mammalian host, especially a human host.

### BACKGROUND OF THE INVENTION

Lentiviral Vectors (LV) provide one of the most efficient vaccine platforms, relied on their outstanding potential of gene transfer to the nuclei of the host cells, including notably Antigen Presenting Cells (APC). Such nuclear transfer of genes initiates expression of antigens which readily access the Major Histocompatibility Complex Class-I (MHC-I) presentation machinery, i.e., proteasome, for further triggering of CD8⁺ T cells. In net contrast with their substantial ability at routing the endogenously produced antigens into the MHC-I pathway, viral vectors, including LV, are barely effective or inoperative in delivery of non-secreted antigens to the endosomal MHC-II compartment (MIIC) and unable to trigger CD4⁺ T cells. Although CD8⁺ T cells contribute largely to the immune control of infectious diseases or tumor growth, CD4⁺ T cells are the major immune players. In addition to their long lifespan and their own direct effector functions, CD4⁺ T cells orchestrate the immune system by regulating innate immunity, tailoring B-cell responses and supporting CD8⁺ T cell effector functions. Therefore, leveraging the potential of LV to induce CD4⁺ T cells will maximize their success rate in vaccine strategies.

### SUMMARY OF THE INVENTION

In one aspect, the present invention relates to a recombinant lentiviral vector genome comprising a polynucleotide encoding a fusion polypeptide, wherein said fusion polypeptide comprises, arranged from N-terminal to C-terminal ends:
a first polypeptide comprising (i) an MHC-II-associated light invariant chain (li), preferably of SEQ ID No. 11, or (ii) the transmembrane domain of the transferrin receptor (TfR), preferably of SEQ ID No. 13, and
at least one antigenic polypeptide of a pathogen.

The present invention further relates to a DNA plasmid comprising the recombinant vector genome according to the invention.

The present invention also relates to a recombinant lentiviral vector or a recombinant lentiviral vector particle which comprises the recombinant lentiviral vector genome according to the invention.

The present invention also relates to a fusion polypeptide which comprises, arranged from N-terminal to C-terminal ends:
a first polypeptide comprising (i) an MHC-II-associated light invariant chain (li), preferably of SEQ ID No. 11, or (ii) the transmembrane domain of the transferrin receptor (TfR), preferably of SEQ ID No. 13, and
at least one antigenic polypeptide of a pathogen.

The present invention also relates to a polynucleotide encoding said polypeptide.

The invention further relates to a host cell, preferably a mammalian host cell, in particular a human host cell, transfected with a DNA plasmid according to the invention, in particular wherein said host cell is a HEK-293T cell line or a K562 cell line.

In another aspect, the invention relates to a pharmaceutical composition, in particular a vaccine composition, suitable for administration to a mammalian host, in particular a human host, comprising a recombinant lentiviral vector of the invention, a recombinant lentiviral vector particle of the invention, or a host cell of the invention together with one or more pharmaceutically acceptable excipient(s) suitable for administration to a host in need thereof, in particular a mammalian host, especially a human host.

In particular, the invention relates to the pharmaceutical composition for use in the elicitation of a protective, preferentially prophylactic, immune response by the elicitation of T-cell responses directed against epitopes contained in the antigenic polypeptide or immunogenic fragments thereof, and/or cellular and/or humoral response in a host in need thereof, in particular a mammalian host, especially a human host.

Another aspect of the invention relates to a method for the preparation of recombinant lentiviral vector particles suitable for the preparation of a pharmaceutical composition, in particular a vaccine composition, comprising the following steps:
a) transfecting the recombinant lentiviral transfer vector carrying the lentiviral vector genome according to the invention, or the DNA plasmid according to the invention in a host cell, for example a HEK-293T cell line or a K562 cell line;
b) co-transfecting the cell of step a) with: (i) a plasmid vector encoding the lentiviral GAG and POL or mutated POL protein as packaging construct; and (ii) a plasmid encoding VSV-G Indiana or New Jersey envelope;
c) culturing the host cell under conditions suitable for the production of recombinant lentiviral vector particles expressing the fusion polypeptide of the invention;
d) recovering the recombinant lentiviral particles expressing the fusion polypeptide of the invention.

### DETAILED DESCRIPTION OF THE INVENTION

The inventors have designed and prepared a platform of lentiviral vector encoding a recombinant fusion protein, in which one or several antigens are fused to a protein domain, generating a membrane-bound protein which traffics through the endosomes, thus delivering the antigen(s) to the MHC-II machinery. The inventors have discovered that MHC-II-pathway-delivering protein domains, in particular the light invariant chain (li) associated with the MHC-II complex, and the transmembrane domain of the transferrin receptor, could elicit an MHC-II antigen presentation and a strong CD4⁺ T-cell immune response, when fused with the antigen(s) of a pathogen, when the antigen is processed into antigen presentation cells expressing MHC-II molecules, using a recombinant lentiviral vector expressing said antigen. This was unexpected, since the T-cell immunogenicity of the existing lentiviral platforms were mostly restricted to a CD8⁺ T-cell immune response.

The inventors have also observed that the MHC-II presentation of the antigen(s) does not show detrimental impact on the MHC-I presentation of the antigen(s) thereby enabling elicitation of an immune response involving both presentation pathways.

The invention hence discloses a recombinant lentiviral vector genome comprising a polynucleotide encoding a fusion polypeptide expressed as a multi-domain recombinant protein comprising an MHC-II-pathway-delivering domain fused with one or several antigenic domains.

The fusion polypeptide is encoded by a polynucleotide that is recombined in the backbone of the lentiviral transfer vector in order to enable preparing lentiviral vector particles expressing the fusion polypeptide harboring the antigen(s) for elicitation of an immunological response, in particular a protective immunogenic response or advantageously a sterile protection against the pathogen providing the antigen(s).

In one aspect, the invention thus relates to a recombinant lentiviral vector genome comprising a polynucleotide encoding a fusion polypeptide, wherein said fusion polypeptide comprises, arranged from N-terminal to C-terminal ends:
a polypeptide comprising (i) an MHC-II-associated light invariant chain (li), preferably of SEQ ID No. 11, or (ii) the transmembrane domain of the transferrin receptor (TfR), preferably of SEQ ID No. 13, and
at least one antigenic polypeptide of a pathogen.

In one embodiment, the fusion polypeptide comprises or consists of a MHC-II-associated light invariant chain (li) fused with at least one antigenic polypeptide of a pathogen.

In another embodiment, the fusion polypeptide comprises or consists of the transmembrane domain of the transferrin receptor (TfR) fused with at least one antigenic polypeptide of a pathogen.

According to the invention, two polypeptides are fused to each other when the nucleotide sequences encoding the two polypeptides are ligated to each other in-frame to create a chimeric gene encoding a fusion polypeptide or protein. In the invention, the nucleotide sequence of the antigenic polypeptide is generally ligated in 3′ position with respect to the nucleotide sequence of the first polypeptide. The fusion between two polypeptide sequences may be direct or indirect. Two polypeptides are fused directly when the C-terminus of the first polypeptide chain is covalently bonded to the N-terminus of the second polypeptide chain. Preferably, the polypeptides are fused indirectly, i.e. a linker or spacer peptide or a further polypeptide is present between the two fused polypeptides.

The invariant chain is preferably the human MHC-II associated light invariant chain. In one embodiment, the light invariant chain comprises, in particular consists of, a sequence of SEQ ID No. 11 or an amino acid sequence with at least 70% amino acid sequence identity, preferably 80% or 85%, preferably 90% or 95% still preferably 98 or 99% with SEQ ID No. 11. In one embodiment, the light invariant chain has 1 to 10, in particular 1 to 5, more particularly 1 to 3 amino acid changes with respect SEQ ID No. 11. As used herein, an amino acid change may consist in an amino acid substitution, addition or deletion. Preferably, the amino acid substitution is a conservative amino acid substitution.

Transferrin receptor naturally acts as a carrier protein for transferrin. Its function is to import iron into the cell by internalizing the transferrin-iron complex through receptor-mediated endocytosis. In the invention, the transferrin receptor is preferably the human transferrin receptor.

Accordingly, the fusion polypeptide may comprise the transmembrane domain of the human transferrin receptor, preferably amino acids 1 to 118 of the human transferrin receptor. In one embodiment, the transmembrane domain of the transferrin receptor, as comprised within the fusion polypeptide of the invention, comprises, preferably consists of, a sequence of SEQ ID No. 13 or an amino acid sequence with at least 70% amino acid sequence identity, preferably 80% or 85%, preferably 90% or 95% still preferably 98 or 99% with SEQ ID No. 13. In one embodiment, the transmembrane domain of the transferrin receptor has 1 to 10, in particular 1 to 5, more particularly 1 to 3 amino acid changes with respect SEQ ID No. 13.

According to the invention, the fusion polypeptide carries one or several antigens.

In an embodiment, the antigenic polypeptide is a mono-antigenic polypeptide comprising one antigen of a pathogen or immunogenic fragment thereof. Alternatively, said antigenic polypeptide a poly-antigenic polypeptide comprising at least two antigens of one or more pathogens or immunogenic fragments thereof.

An “antigen” or an “antigenic polypeptide” as defined herein as a wild type or native antigen of a pathogenic organism or as a fragment of such wild type a native antigen or as a mutated polypeptide comprising less than 5% of mutated especially substituted amino acid residues with respect to the wild type or native antigen. Mutations are in particular point mutations of 1, 2, 3 or 4 amino acid residues of the amino acid sequence of the wild type or native antigen. A fragment of the wild type or the native antigen advantageously keeps the immunogenic properties of the polypeptide from which it derives or shows improved immunogenic properties when it is expressed by the lentiviral vector of the invention and advantageously shows immune protective properties when expressed in a host. A fragment of an antigen has an amino acid sequence which is sufficient to provide one or several epitope(s) in particular T-cell epitopes and more particularly CD4+ or CD8+ T-cell epitopes or both and which keeps the immunogenic, especially the protective properties leading to the protective activity of the antigenic polypeptide from which it derives and/or exhibits such protective properties when expressed by the lentiviral vector of the invention.

The expression “T-cell epitope” refers to antigenic determinants that are involved in the adaptive immune response driven by T cells. In particular said T-cell epitopes elicit T cells, when delivered to the host in suitable conditions. According to a particular embodiment the antigenic polypeptides targeted according to the invention and the polypeptide derivatives of these antigenic polypeptides comprise epitope(s) mediating CD4+ T-cell response and advantageously also epitope(s) mediating CD8⁺ T-cell response.

Polypeptides and antigens described and used in the invention may have at least 50% amino acid identity with the native protein, in particular at least 60%, in particular at least 70%, in particular at least 80%, more particularly at least 90 or 95%, more particularly at least 99% identity.

In a particular embodiment the fusion polypeptide provides at least 2, in particular at least 3 or at least 4 or at least 5 and in particular are especially 2, 3, 4 or 5, and accordingly encompass at least 2, at least 3 or at least 4 antigens (and/or antigenic fragments or mutated antigens with respect to a native or wild type determined antigen of a pathogen). In a particular embodiment the antigenic polypeptide contained in the fusion polypeptide comprises or consists of a fusion of up to 6 antigens or antigenic fragments or mutated fragments thereof. The inventors have demonstrated that the fusion polypeptide of the invention is capable of driving the expression of large antigenic polypeptides, fused behind the first polypeptide. In one embodiment, the fusion polypeptide comprises at least 200 amino acids, in particular at least 300 amino acids, in particular at least 400 amino acids, more particularly at least 500 or 600 amino acids. In one embodiment, the fusion polypeptide comprises from 200 to 1000 amino acids, in particular from 200 to 800 amino acids. In one embodiment, the antigenic polypeptide, comprising the one or several antigens expressed by the lentiviral vector, comprises at least 100 amino acids, in particular at least 300 amino acids, more particularly at least 400 or 500 amino acids. In one embodiment, the antigenic polypeptide comprises from 100 to 1000 amino acids, in particular from 200 to 600 amino acids.

The antigenic polypeptide may be fused to the first polypeptide via a linker. Similarly, when several antigen(s) or immunogenic fragments thereof are present within the fusion polypeptide, the sequences of the antigens may be separated by linker sequences, to avoid the formation of neo-epitopes. In particular, peptide linkers may be used, such as four amino acid linkers GGGD, NNGG or NNDD. Suitable linkers are also shown in the Examples, in particular in Table S3.

Preferably, the one or more antigens are selected and arranged within the fusion polypeptide such as to preserve the native tertiary structure of the antigen(s) when the fusion polypeptide is expressed. By preserving the native protein folding, the lentiviral vector can induce efficient antigen routing to the MHC-II machinery.

In one embodiment, the pathogen is selected from a bacterial, parasite or viral pathogen, in particular a pathogen infecting mammals or human hosts or is a tumoral antigen or immunogenic fragment thereof, in particular an antigen from a mammalian tumor, especially a human tumor or an immunogenic fragment thereof. In one embodiment, the fusion polypeptide comprises at least two antigens or immunogenic fragments thereof, wherein the at least two antigens or immunogenic fragments thereof are selected from the same of from distinct pathogens. In one embodiment, the pathogen is associated with an acute or a chronic respiratory infectious disease and in particular may be selected from Mycobacterium tuberculosis (Mtb), an influenza virus in particular a type A, type B or type C influenza virus, more specifically an H1N1, H2N2 or H3N2 influenza virus, or a coronavirus, in particular SARS-COV-2.

In particular, the antigenic polypeptide may comprise one or more Mycobacterium tuberculosis (Mtb) antigens, in particular selected from EsxA (UniProtKB-P9WNK7), EspC (UniProtKB-P9WJD7), EsxH (UniProtKB-P9WNK3), PE19 (UniProtKB-Q79FK4), or Ag85A (UniProtKB-P9WQP3), or (an) immunogenic fragment(s) thereof, in particular a fragment lacking the initial methionine. Preferably, the immunogenic fragment of EsxH comprises the MHC epitope of SEQ ID No. 15 and/or the MHC epitope of SEQ ID No. 16. Preferably, the immunogenic fragment of EsxA comprises the MHC epitope of SEQ ID No. 17. Preferably, the immunogenic fragment of PE19 comprises the MHC epitope of SEQ ID No. 18. Preferably, the immunogenic fragment of Ag85A comprises the MHC epitope of SEQ ID no. 19. In one embodiment, the antigenic polypeptide may comprise one of the following Mtb antigenic combinations:
(a) EsxH;
(b) EsxH and EsxA;
(c) EsxH, EsxA and PE19;
(d) EsxH, EsxA, EspC and PE19;
(e) EsxH, EsxA, EspC, PE19 and Ag85A;
or immunogenic fragments thereof, especially a combination of immunogenic fragments encompassing the MHC epitopes mentioned herein.

In one embodiment, the antigenic polypeptide and/or the fusion polypeptide containing the antigenic polypeptide does not comprise the sequence of ovalbumin or an immunogenic fragment thereof.

In one embodiment, the fusion polypeptide has the sequence set forth in SEQ ID No. 24, wherein the sequence of the antigenic polypeptide may be replaced by another antigenic polypeptide of interest.

The invention also relates to the fusion polypeptide as defined herein.

The invention further relates to a nucleic acid molecule encoding the fusion polypeptide defined herein. The nucleic acid may be DNA, in particular cDNA or may be RNA, in particular stabilized RNA. The RNA sequences are deducted from the DNA sequences wherein the Thymine (T) nucleobase is replaced by an Uracile (U) nucleobase. RNA polynucleotides may be obtained by transcription of DNA or cDNA or may be synthesized.

The nucleic acid molecule may further comprise control nucleotide sequences for the transcription or for the expression of the fusion polypeptide comprising the antigen(s). It may also be modified, in order to be operably ligated to a distinct polynucleotide such as a plasmid or a vector genome (transfer plasmid), in particular a lentiviral vector genome. It may also be modified, in particular to be rendered more stable such as for use as RNA. In a further embodiment, the nucleic acid is a mammalian codon-optimized, in particular a human codon-optimized sequence for expression in mammalian, respectively human cells.

The invention also relates to a plasmid vector recombined with a nucleic acid molecule encoding the fusion polypeptide carrying antigen(s) selected for the elicitation of an immune response in a host.

In an embodiment, the plasmid vector is a transfer vector in particular a lentiviral transfer vector suitable to provide the genome of a lentiviral vector of the invention. The lentiviral vector expresses the selected antigenic polypeptide(s) within their fusion polypeptide when expressed in vivo in a host.

In a particular embodiment, the nucleic acid molecule containing the genome of the transfer vector is provided as a plasmid comprising the lentiviral backbone vector recombined with a polynucleotide encoding the selected antigen(s) of the pathogen, for their expression as a fusion polypeptide when said vector genome is provided in a lentiviral vector particle that is used for administration to a host.

Additionally, the nucleic acid molecule may contain sequences for the control of transcription and/or for the control of expression, and/or may contain sequences for ligation to a distinct nucleic acid such as for ligation to a plasmid or a vector genome. Hence the nucleic acid may contain one or more sequences for restriction site(s), Kozak sequence, promoter or other sequences as disclosed herein and illustrated in the examples.

The expression “vector” relates to biological or chemical entities suitable for the delivery of the polynucleotides encoding the polypeptides of the invention to the cells of the host administered with such vectors. Vectors are well known in the art and may be viral vectors as those described herein such as lentiviruses which infect human. The invention relates in particular to the use of HIV vectors, especially HIV-1 vectors which are illustrated in the Examples. Details for the construction for HIV-1 vectors are known in the art and provided in the examples.

In accordance with the invention, lentiviral vectors expressing antigenic polypeptides are provided wherein the vectors have or comprise in their genome (vector genome) a recombinant polynucleotide which encodes a fusion polypeptide according to the invention, wherein said fusion polypeptide comprises at least one antigenic polypeptide, in particular of a pathogen.

The lentiviral vectors of the invention, especially the preferred HIV-1 based vectors, may be replication-incompetent pseudotyped lentiviral vectors, in particular a replication-incompetent pseudotyped HIV-1 lentiviral vector, wherein said vector contains a genome comprising a mammal codon-optimized synthetic nucleic acid, in particular a human-codon optimized synthetic nucleic acid, wherein said synthetic nucleic acid encodes a fusion polypeptide according to the invention, comprising (an) antigenic polypeptide(s), in particular the antigenic polypeptide(s) of a determined pathogen infecting a mammal, in particular a human host. The lentiviral vector may be pseudotyped with the glycoprotein G from a Vesicular Stomatitis Virus (V-SVG) of Indiana or of New-Jersey serotype.

Use of codon-optimized sequences in the genome of the vector particles allows in particular strong expression of the antigenic polypeptide in the cells of the host administered with the vector, especially by improving mRNA stability or reducing secondary structures. In addition, the expressed antigenic polypeptide undergoes post translational modifications which are suitable for processing of the antigenic polypeptide in the cells of the host, in particular by modifying translation modification sites (such as glycosylation sites) in the encoded polypeptide. Codon optimization tools are well known in the art, including algorithms and services such as those made available by GeneArt (Life technologies-USA) and DNA2.0 (Menlo Park, California-USA). In a particular embodiment codon-optimization is carried out on the open reading frame (ORF) sequence encoding the antigenic polypeptide and the optimization is carried out prior to the introduction of the sequence encoding the ORF into the plasmid intended for the preparation of the vector genome. In another embodiment, additional sequences of the vector genome are also codon-optimized.

The active ingredients consisting of the viral vectors may be integrative pseudotyped lentiviral vectors, especially replication-incompetent integrative pseudotyped lentiviral vectors, in particular a HIV-1 vector. Such lentiviral vectors may in addition contain a genome comprising a mammal-codon optimized synthetic nucleic acid, in particular a human-codon optimized synthetic nucleic acid, wherein said synthetic nucleic acid encodes a fusion polypeptide according to the invention, comprising (an) antigenic polypeptide(s), in particular the antigenic polypeptide(s) of a determined pathogen infecting a mammal such as disclosed herein, in particular a virus or a bacteria or a parasite infecting a human host.

Alternatively, the lentiviral vector and in particular the HIV-1 based vector may be a non-integrative replication-incompetent pseudotyped lentiviral vector.

A particular embodiment of a lentiviral vector suitable to achieve the invention relates to a lentiviral vector whose genome is obtained from the pTRIP vector plasmid or the pFLAPdeltaU3 vector plasmid, preferably the pFLAPdeltaU3 plasmid, in particular the vector plasmid of nucleotide sequence SEQ ID No. 20, wherein the nucleic acid encoding the fusion polypeptide has been cloned under control of a promoter functional in mammalian cells, in particular the CMV promoter, the human β2-microglobulin promoter, the SP1-β2m promoter of SEQ ID No. 21 or the composite “BCUAG” promoter of SEQ ID No. 22, preferably the SP1-β2m promoter, and wherein the vector optionally comprises post-transcriptional regulatory element of the woodchuck hepatitis virus (WPRE), wild type or mutated. In particular, the WPRE is a mutant WPRE as set forth in SEQ ID No. 23.

The pFLAPdeltaU3 plasmid or pFLAP plasmid, is a lentiviral plasmid vector derived from the pTRIP plasmid. Examples of pFLAP plasmids are shown in FIGS. 13, 14 and 15.

In a further embodiment of the invention, the lentiviral vector particle expressing the fusion polypeptide according to the features herein described is pseudotyped with the glycoprotein G from a Vesicular Stomatitis Virus (V-SVG) of Indiana or of New-Jersey serotype.

The particular features of such lentiviral vectors will be further discussed in detail below.

The invention also relates to a DNA plasmid comprising the recombinant lentiviral vector genome according to the definitions provided herein, in particular wherein said genome is inserted within the pFLAPdeltaU3 vector plasmid, preferably the vector plasmid of nucleotide sequence SEQ ID No. 20, wherein the fusion polypeptide according to the invention is inserted between restriction sites BamHI and Xhol in replacement of the GFP sequence.

The invention further relates to a host cell, preferably a mammalian host cell, comprising the lentiviral vector genome of the invention, or transfected with a DNA plasmid according to the invention. In particular, said host cell is a HEK-293T cell line or a K562 cell line. The invention further relates to a culture of said host cells.

The invention also relates to a formulation or pharmaceutical composition, in particular a vaccine composition, suitable for administration to a mammalian host, comprising a recombinant lentiviral vector of the invention together with one or more pharmaceutically acceptable excipient(s) suitable for administration to a host in need thereof, in particular a mammalian host, especially a human host.

The invention also relates to a formulation suitable for administration to a mammalian host, in particular a human host comprising as an active ingredient lentiviral vector particles as defined herein for protection against a pathogen infection or against the pathogen-induced condition or disease, together with excipient(s) suitable for administration to a host in need thereof, in particular a human host. The disease may be an acute or a chronic respiratory infectious disease such as tuberculosis, influenza, in particular caused by a type A, type B or type C influenza virus, more specifically an H1N1, H2N2 or H3N2 influenza virus. The disease may also be a coronavirus disease, in particular caused by SARS-COV-2.

The pharmaceutical composition, in particular the vaccine composition, or the formulation according to the invention may also comprise an adjuvant component, in particular a pro-Th1 and/or pro-Th17 adjuvant, and/or an immunostimulatory component.

In particular, the composition or formulation may comprise a pro-Th1 adjuvant such as polyinosinic-polycytidylic acid (polyI:C) or a derivative thereof. A derivative of poly (I:C) refers to a mismatched dsRNA obtained by modifying the specific configuration of poly (I:C) through the introduction of unpaired bases thereinto, and includes poly (I:CxU), poly (IxU:C) (where x is on average a number from 3 to 40) and the like. Preferably, a derivative of poly (I:C) is poly (I:C12U) or poly (C:I12U), which is commercially available under the trade name Ampligen™.

The composition or formulation may also comprise a pro-Th1/Th17 adjuvant such as a cyclic dinucleotide adjuvant. Cyclic nucleotide adjuvants are also referred to as STING-activating cyclic dinucleotide adjuvant. The term “cyclic dinucleotides” (“CDNs”) as used herein refers to a class of molecules comprising 2′-5′ and/or 3′-5′ phosphodiester linkages between two purine nucleotides. This includes 2′-5′-2′,5′, 2′-5′-3′5′, and 3′,5′-3′,5′ linkages. CDNs are ubiquitous small molecule second messengers synthesized by bacteria that regulate diverse processes and are a relatively new class of adjuvants that have been shown to increase vaccine potency. CDNs activate innate immunity by directly binding the endoplasmic reticulum-resident receptor STING (stimulator of interferon genes), activating a signaling pathway that induces the expression of interferon-β (IFN-β) and also nuclear factor-KB (NF-κB) dependent inflammatory cytokines. Preferably, the CDN is cyclic Guanine-Adenine dinucleotide (cGAMP).

The inventors have shown that the use of adjuvants, in particular pro-Th1 and/or pro Th17 adjuvants, together with the lentiviral vector of the invention, elicited the generation of Th1 CD4⁺ or CD8⁺ T cells, as well as IL-17A-producing Th17 CD4⁺ T cells.

In another aspect of the invention, the active ingredient, in particular the lentiviral vector particles, or the composition or the formulation comprising the same is for use in the protective immunization against a pathogenic infection or against pathogen-induced condition or disease, in a mammalian host, especially a human host, optionally in association with an appropriate delivery vehicle and optionally with an adjuvant component and/or with an immunostimulant component, e.g. an adjuvant component and/or immunostimulant component as defined in the present specification.

Accordingly, the active ingredient, or the composition, in particular the lentiviral vector particles of the invention, when administered to a host in need thereof, especially to a mammalian, in particular to a human host, elicits an immune response by the elicitation of antibodies directed against the antigenic polypeptide or immunogenic fragments thereof. Said immune response may encompass activation of naïve lymphocytes and generation of effector T-cell response and generation of immune memory antigen-specific T-cell response against antigen(s) of the pathogen.

One aspect of the invention relates to the active ingredient, in particular the lentiviral vector particles, the pharmaceutical composition and/or formulation of the invention, for use in preventing and/or treating an infection by a pathogen in a mammalian host in need thereof, in particular a human host in particular an infection by a pathogen associated with an acute or chronic respiratory infectious disease in a mammal. The invention also relates to a method of preventing and/or treating an infection by a pathogen in a mammalian host in need thereof, in particular a human host in particular an infection by a pathogen associated with an acute or chronic respiratory infectious disease in a mammal, wherein said method comprises administering an effective dose of the active ingredient, pharmaceutical composition and/or formulation of the invention to said mammalian host.

The products, methods and uses described herein may be for human or veterinary applications.

The immune response involves the induction of MHC-I restricted presentation and MHC-II restricted presentation of the antigenic polypeptide or immunogenic fragments thereof, by an antigen-presenting cell, in particular a dendritic cell, and the induction of a CD4- and CD8-mediated immune response. By way of contrast, a comparable vector using the same antigenic polypeptide not fused to the invariant chain (li) or the transmembrane domain of the transferrin receptor, does not trigger a significant CD4+ T-cell response. The robust CD4+ T-cell response elicited by the lentiviral vector of the invention is thus unexpected and overcomes the drawbacks encountered using the existing lentiviral vectors, which are restricted to a CD8+ T-cell response. In one embodiment, the CD4+ T-cell response elicited by a lentiviral vector of the invention is at least 30% higher, preferably at least 50% higher, still preferably at least 100% higher, even preferably at least 200% higher, in comparison with a comparable lentiviral vector in which the antigenic polypeptide(s) is expressed alone and not within a fusion protein, fused to the invariant chain (li) or the transmembrane domain of the transferrin receptor. The CD4+ T-cell response may be measured by assessing the expansion of antigen-specific CD4+ T cells in response to an administration, e.g. an injection, of the lentiviral vector of the invention, preferably in a pharmaceutical composition, in particular vaccine composition. The Examples of the present specification illustrate such as measurement.

The lentiviral vector of the invention is particularly capable of eliciting the generation of IFN-γ/TNF-α-producing CD4+ or CD8+ T cells.

The immune response may either prevent the infection by the pathogen or may prevent the onset or the development of a pathological state resulting from infection.

Physiologically acceptable vehicles may be chosen with respect to the administration route of the immunization composition. In a preferred embodiment, administration may be carried out by injection, in particular intramuscularly, intradermally, subcutaneously, or, by intranasal administration or topical skin application.

Recombinant lentiviral vector particles of the invention are used for elicitation in a host, in particular a mammalian host, especially a human host, of an immune response against the pathogen providing the antigens expressed by the particles, said use involving an immunization pattern comprising administering an effective amount of an active ingredient, in particular the lentiviral particles that elicits the cellular immune response of the host as a prime, and later in time administering an effective amount of the same active ingredient or another active ingredient, e.g. the lentiviral particles, to boost the cellular immune response of the host, and optionally repeating (once or several times) said administration step for boosting.

For each step of administration of the lentiviral vector particles, in particular in a regimen that encompasses multiple administration steps, it is preferred that the pseudotyping envelope protein(s) of the vector particles is (are) different from the one used in the other step(s), especially originate from different viruses, in particular different VSVs. In the prime-boost regimen, the administered combination of compounds of each step comprises lentiviral vectors as defined herein.

Priming and boosting steps are separated in time by at least 2 weeks, in particular 6 weeks, in particular by at least 8 weeks.

In a particular embodiment, the recombinant lentiviral vector particles of the invention are used for elicitation in a host, in particular a mammalian host, especially a human host, of an immune response against the pathogen providing the antigens expressed by the particles, said use involving an immunization pattern comprising a heterologous prime-boost regimen wherein the recombinant lentiviral vector particles of the invention are used for a boost. The priming step may be performed using a live-attenuated pathogen vaccine or another heterologous immunogenic composition with respect to the recombinant lentiviral vector particles of the invention. Details on the administration regimen will be discussed further below.

The LV particles provides a cellular immune response (T-cell immune response), particularly a CD4+ T-cell immune response and advantageously a CD8+-T-cell immune response, i.e., an adaptive immune response which is mediated by activated cells harbouring respectively CD4 or CD8 receptors.

In a particularly advantageous embodiment, the immune response conferred by the LV particles, is a long-lasting immune response i.e., said immune response encompasses memory cells response and in particular central memory cells response; in a particular embodiment it can be still detected at least several months after the last administration step.

In accordance with the invention when the lentiviral particles are used in a prime-boost regimen or a multiple steps administration regimen, lentiviral vector particles are provided which are pseudotyped with a first determined pseudotyping envelope G protein obtained from the VSV, strain Indiana or New-Jersey, and later administered lentiviral vector particles are provided which are pseudotyped with a second determined pseudotyping envelope G protein obtained from a VSV, strain New Jersey or Indiana. The order of use in the prime-boost regimen of the first and second compounds thus described may alternatively be inversed. Thus, the lentiviral vector particles contained in the separate active ingredients/compounds of the combinations or compositions of the invention when intended for use in a prime-boost regimen are distinct from each other, at least due to the particular pseudotyping envelope protein(s) used for pseudotyping the vector particles.

Doses of lentiviral vectors intended for elicitation of the cellular immune response which are used in the administration pattern, may comprise from 10⁵ TU to 10¹⁰ TU of recombinant lentiviral particles especially from 10⁵ to 10⁸ TU, when integrative vectors are used. The dose intended for administration to the host may comprise from 10⁸ to 10¹⁰ of each type of recombinant lentiviral vector particles when integrative-incompetent vectors are used.

The invention also concerns a method of providing immunization in a mammalian host, especially in a human host, comprising the step of administering, as a prime or as a boost, the recombinant lentiviral vector particles of the invention to elicit the immune response, and optionally repeating the administration steps one or several times, in particular to boost said response, in accordance with the present disclosure.

Optionally, the recombinant lentiviral vector particles may be used in association with an adjuvant compound suitable for administration to a mammalian, especially a human host, and/or with an immunostimulant compound, together with an appropriate delivery vehicle. Suitable adjuvants and immunostimulant compounds are described in the present specification.

The recombinant lentiviral vector particles can be administered to the host via injection through different routes including subcutaneous (s.c.), intradermal (i.d.), intramuscular (i.m.) or intravenous (i.v.) injection, or may be administered orally to topically trough mucosal or skin administration, especially intranasal (i.n.) administration or inhalation. The quantity to be administered (dosage) depends on the subject to be treated, including considering the condition of the patient, the state of the individual's immune system, the route of administration and the size of the host. Suitable dosages range may be determined with respect to the content in equivalent transducing units of HIV-1-derived lentiviral vector particles.

Other examples and features of the invention will be apparent when reading the examples and the figures which illustrate the preparation and application of the lentiviral vector particles with features that may be individually combined with the definitions given in the present description.

### Detailed Description of the Lentiviral Vectors for Use According to the Invention

The invention accordingly involves lentiviral vectors which are recombinant lentiviral particles (i.e. recombinant vector particles), and which may be replication-incompetent lentiviral vectors, especially replication-incompetent HIV-1 based vectors characterized in that: (i) they are pseudotyped with a determined heterologous viral envelope protein or viral envelope proteins originating from a RNA virus which is not HIV, and (ii) they comprise in their genome at least one recombinant polynucleotide encoding a fusion polypeptide of the invention, comprising at least one antigenic polypeptide (or polypeptide derivative thereof such as immunogenic fragment(s) thereof) carrying epitope(s) of an antigen of a pathogen wherein the pathogen is capable of infecting a mammalian host, in particular a human host and wherein said epitopes encompass T-cell epitope(s), in particular both CD4+ T-cell epitopes and CD8+ T-cell epitopes.

According to a particular embodiment of the invention, the lentiviral vectors are either designed to express proficient (i.e., integrative-competent) or deficient (i.e., integrative-incompetent) particles. According to a particular embodiment of the invention, the recombinant lentiviral vector particles are both integration-incompetent and replication-incompetent.

The preparation of the lentiviral vectors is well known from the skilled person and has been extensively disclosed in the literature (confer for review Sakuma T. et al (Biochem. J. (2012) 443, 603-618). The preparation of such vectors is also illustrated herein in the Examples.

In a particular embodiment of the invention, the polynucleotide(s) encoding the antigenic polypeptides (ORF) of the lentiviral vector has (have) been mammal-codon optimized (CO) in particular human-codon optimized. Optionally the lentiviral sequences of the genome of said particles have also a mammal-codon optimized nucleotide sequence. In a particular aspect of the invention the codon optimization has been carried out for expression in mouse cells. In another embodiment the sequence of the polynucleotide(s) encoding the antigenic polypeptides of the lentiviral vector has (have) been human-codon optimized (CO).

It has been observed that codon optimized nucleotide sequences, especially when optimized for expression in mammalian and in particular in human cells, enable the production of higher yield of particles in such mammalian or human cells. Production cells are illustrated in the examples. Accordingly, when lentiviral vector particles of the invention are administered to a mammalian, especially to a human host, higher amounts of particles are produced in said host which favour the elicitation of a strong immune response.

The recombinant lentiviral vector (i.e., lentiviral vectors particles or lentiviral-based vector particles) defined in the present invention are pseudotyped lentiviral vectors consisting of vector particles bearing envelope protein or envelope proteins which originate from a virus different from the particular lentivirus (especially a virus different from HIV, in particular HIV-1), which provides the vector genome of the lentiviral vector particles. Accordingly, said envelope protein or envelope proteins, are “heterologous” viral envelope protein or viral envelope proteins with respect to the vector genome of the particles. In the following pages, reference will also be made to “envelope protein(s)” to encompass any type of envelope protein or envelope proteins suitable to perform the invention.

When reference is made to “lentiviral” vectors (lentiviral-based vectors) in the application, it relates in particular, to HIV-based vectors and especially HIV-1-based vectors.

The lentiviral vectors suitable to perform the invention are so-called replacement vectors, meaning that the sequences of the original lentivirus encoding the lentiviral proteins are essentially deleted in the genome of the vector or, when present, are modified, and especially mutated, especially truncated, to prevent expression of biologically active lentiviral proteins, in particular, in the case of HIV, to prevent the expression by said transfer vector providing the genome of the recombinant lentiviral vector particles, of functional ENV, GAG, and POL proteins and optionally of further structural and/or accessory and/or regulatory proteins of the lentivirus, especially of HIV.

In a particular embodiment, the lentiviral vector is built from a first-generation vector, in particular a first-generation of a HIV-based vector which is characterized in that it is obtained using separate plasmids to provide (i) the packaging construct, (ii) the envelope and (iii) the transfer vector genome. Alternatively, it may be built from a second-generation vector, in particular a second-generation of a HIV-based vector which in addition, is devoid of viral accessory proteins (such as in the case of HIV-1, Vif, Vpu, Vpr or Nef) and therefore includes only four out of nine HIV full genes: gag, pol, tat and rev. In another embodiment, the vector is built from a third-generation vector, in particular a third-generation of a HIV-based vector which is furthermore devoid of said viral accessory proteins and also is Tat-independent; these third-generation vectors may be obtained using 4 plasmids to provide the functional elements of the vector, including one plasmid encoding the Rev protein of HIV when the vector is based on HIV-1. Such vector system comprises only three of the nine genes of HIV-1. The structure and design of such generations of HIV-based vectors is well known in the art.

In any of these generations of the vector, modifications are additionally provided according to the invention by insertion in the vector backbone of the fusion polypeptide as described herein, to provide a LV vector leveraged to target and activate APC, in particular dendritic cells to route immunogens to MHC-II pathway and to induce both CD4+ and CD8+ T-cell responses.

The “vector genome” of the vector particles is a recombinant nucleic acid which also comprises as a recombined sequence the polynucleotide or transgene of interest encoding the fusion polypeptide according to the invention comprising one or more antigenic polypeptide(s) or immunogenic fragment(s) thereof, in particular of pathogen as disclosed herein. The lentiviral-based sequence and polynucleotide/transgene of the vector genome are borne by a plasmid vector thus giving rise to the “transfer vector” also referred to as “sequence vector”. Accordingly, these expressions are used interchangeably in the present description. According to a particular embodiment, a vector genome prepared for the invention comprises a nucleic acid having a sequence of SEQ ID No. 20, in which the polynucleotide encoding the fusion polypeptide of the invention is inserted between restriction sites BamHI and Xhol in replacement of GFP sequence (SEQ ID No. 30).

The vector genome as defined herein accordingly contains, apart from the so-called recombinant polynucleotide(s) encoding the fusion polypeptide of the invention comprising the antigenic polypeptide(s) placed under control of proper regulatory sequences for its expression, the sequences of the original lentiviral genome which are non-coding regions of said genome, and are necessary to provide recognition signals for DNA or RNA synthesis and processing (mini-viral genome). These sequences are especially cis-acting sequences necessary for packaging (w), reverse transcription (LTRs possibly mutated with respect to the original ones) and transcription and optionally integration (RRE) and furthermore for the particular purpose of the invention, they contain a functional sequence favouring nuclear import in cells and accordingly transgene transfer efficiency in said cells, which element is described as a DNA Flap element that contains or consists of the so-called central cPPT-CTS nucleotidic domain present in lentiviral genome sequences especially in HIV-1 or in some retroelements such as those of yeasts.

The structure and composition of the vector genome used to prepare the lentiviral vectors of the invention are based on the principles described in the art and on examples of such lentiviral vectors primarily disclosed in (Zennou et al, 2000; Firat H. et al, 2002; VandenDriessche T. et al). Constructs of this type have been deposited at the CNCM (Institut Pasteur, France) as will be referred to herein. In this respect reference is also made to the disclosure, including to the deposited biological material, in patent applications WO 99/55892, WO 01/27300 and WO 01/27304.

According to a particular embodiment of the invention, a vector genome may be a replacement vector in which all the viral protein coding sequences between the 2 long terminal repeats (LTRs) have been replaced by the recombinant polynucleotide encoding the fusion polypeptide of the invention comprising the antigenic polypeptide(s) as disclosed herein, and wherein the DNA-Flap element has been re-inserted in association with the required cis-acting sequences described herein. Further features relating to the composition of the vector genome are disclosed in relation to the preparation of the particles.

In a particular embodiment, a lentiviral vector of the invention may comprise in its genome one or more than one recombinant polynucleotide encoding a fusion polypeptide according to the invention. In particular, said vector genome comprises two polynucleotides which are consecutive or separated on the genome and which encode different polypeptides of either the same or distinct antigens of the pathogen or of distinct pathogens.

Particular features of the lentiviral vectors used in accordance with the various embodiments of the invention are also disclosed in the Examples, such features being either taken alone or in combination to produce the vectors.

According to an embodiment of the invention, the lentiviral vector particles are pseudotyped with a heterologous viral envelope protein or viral polyprotein of envelope originating from an RNA virus which is not the lentivirus providing the lentiviral sequences of the genome of the lentiviral particles.

As examples of typing envelope proteins for the preparation of the lentiviral vector, the invention relates to viral transmembrane glycosylated (so-called G proteins) envelope protein(s) of a Vesicular Stomatitis Virus (VSV), which is (are) for example chosen in the group of VSV-G protein(s) of the Indiana strain and VSV-G protein(s) of the New Jersey strain.

Other examples of VSV-G proteins that may be used to pseudotype the lentiviral vectors of the invention encompass VSV-G glycoprotein may especially be chosen among species classified in the vesiculovirus genus: Carajas virus (CJSV), Chandipura virus (CHPV), Cocal virus (COCV), Isfahan virus (ISFV), Maraba virus (MARAV), Piry virus (PIRYV), Vesicular stomatitis Alagoas virus (VSAV), Vesicular stomatitis Indiana virus (VSIV) and Vesicular stomatitis New Jersey virus (VSNJV) and/or stains provisionally classified in the vesiculovirus genus as Grass carp rhabdovirus, BeAn 157575 virus (BeAn 157575), Boteke virus (BTKV), Calchaqui virus (CQIV), Eel virus American (EVA), Gray Lodge virus (GLOV), Jurona virus (JURV), Klamath virus (KLAV), Kwatta virus (KWAV), La Joya virus (LJV), Malpais Spring virus (MSPV), Mount Elgon bat virus (MEBV), Perinet virus (PERV), Pike fry rhabdovirus (PFRV), Porton virus (PORV), Radi virus (RADIV), Spring viremia of carp virus (SVCV), Tupaia virus (TUPV), Ulcerative disease rhabdovirus (UDRV) and Yug Bogdanovac virus (YBV).

The envelope glycoprotein of the vesicular stomatitis virus (VSV-G) is a transmembrane protein that functions as the surface coat of the wild type viral particles. It is also a suitable coat protein for engineered lentiviral vectors. Presently, nine virus species are definitively classified in the VSV gender, and nineteen rhabdoviruses are provisionally classified in this gender, all showing various degrees of cross-neutralisation. When sequenced, the protein G genes indicate sequence similarities.

The VSV-G protein presents an N-terminal ectodomain, a transmembrane region and a C-terminal cytoplasmic tail. It is exported to the cell surface via the trans-Golgi network (endoplasmic reticulum and Golgi apparatus).

Vesicular stomatitis Indiana virus (VSIV) and Vesicular stomatitis New Jersey virus (VSNJV) are preferred strains to pseudotype the lentiviral vectors of the invention, or to design recombinant envelope protein(s) to pseudotype the lentiviral vectors. Their VSV-G proteins are disclosed in GenBank, where several strains are presented. For VSV-G New Jersey strain reference is especially made to the sequence having accession number V01214. For VSV-G of the Indiana strain, reference is made to the sequence having accession number AAA48370.1 in Genbank corresponding to strain JO2428.

Said viral envelope protein(s) are capable of uptake by antigen presenting cells and especially by dendritic cells including by liver dendritic cells by mean of fusion and/or of endocytosis. In a particular embodiment, the efficiency of the uptake may be used as a feature to choose the envelope of a VSV for pseudotyping. In this respect the relative titer of transduction (Titer DC/Titer of other transduced cells e.g. 293T cells) may be considered as a test and envelope having a relatively good ability to fuse with DC would be preferred.

Antigen Presenting Cells (APC) and especially Dentritic cells (DC) are proper target cells for pseudotyped lentiviral vectors which are used as immune compositions accordingly.

The VSV-G envelope protein(s) are expressed from a polynucleotide containing the coding sequence for said protein(s), which polynucleotide is inserted in a plasmid (designated envelope expression plasmid or pseudotyping env plasmid) used for the preparation of the lentiviral vector particles of the invention. The polynucleotide encoding the envelope protein(s) is under the control of regulatory sequences for the transcription and/or expression of the coding sequence including optionally post-transcriptional regulatory elements (PRE) especially a polynucleotide such as the element of the Woodchuck hepatitis virus, i.e. the WPRE sequence, obtainable from Invitrogen or a mutant sequence of WPRE as set forth in SEQ ID No. 23.

Accordingly, a nucleic acid construct is provided which comprises an internal promoter suitable for the use in mammalian cells, especially in human cells in vivo and the nucleic acid encoding the envelope protein under the control of said promoter. A plasmid containing this construct is used for transfection of cells suitable for the preparation of vector particles. Promoters may in particular be selected for their properties as constitutive promoters, tissue-specific promoters, or inducible promoters. Examples of suitable promoters encompass the promoters of the following genes: MHC Class-I promoters, human beta-2 microglobulin gene (B2M promoter), EF1α, human PGK, PPI (preproinsulin), thiodextrin, HLA DR invariant chain (P33), HLA DR alpha chain, Ferritin L chain or Ferritin H chain, Chymosin beta 4, Chymosin beta 10, Cystatin Ribosomal Protein L41, CMVie or chimeric promoters such as GAG (CMV early enhancer/chicken β actin) disclosed in Jones S. et al (Jones S. et al Human Gene Therapy, 20:630-640 (June 2009)) or beta-2m-CMV (BCUAG) as disclosed herein.

These promoters may also be used in regulatory expression sequences involved in the expression of gag-pol derived proteins from the encapsidation plasmids, and/or to express the antigenic polypeptides from the transfer vector.

Alternatively, when the envelope expression plasmid is intended for expression in stable packaging cell lines, especially for stable expression as continuously expressed viral particles, the internal promoter to express the envelope protein(s) is advantageously an inducible promoter such as one disclosed in Cockrell A. S. et al. (Mol. Biotechnol. (2007) 36:184-204). As examples of such promoters, reference is made to tetracycline and ecdysone inducible promoters. The packaging cell line may be the STAR packaging cell line (ref Cockrell A. S. et al (2007), Ikedia Y. et al (2003) Nature Biotechnol. 21:569-572) or a SODk packaging cell line, such as SODK0 derived cell lines, including SODk1 and SODk3 (ref Cockrell A. S. et al (2007), Cockrell A S. et al (2006) Molecular Therapy, 14:276-284, Xu K. et al. (2001), Kafri T. et al (1999) Journal of Virol. 73:576-584).

According to the invention, the lentiviral vectors are the product recovered from co-transfection of mammalian cells, with:
a vector plasmid comprising (i) lentiviral, especially HIV-1, cis-active sequences necessary for packaging, reverse transcription, and transcription and further comprising a functional lentiviral, especially derived from HIV-1, DNA flap element and (ii) a polynucleotide encoding the fusion polypeptide of the invention, itself comprising one or more antigenic polypeptide(s) or immunogenic fragment(s) thereof of one or more pathogens against which an immune response is sought under the control of regulatory expression sequences, preferably a human 32 microglobulin promoter or a modified human B2-microglobulin promoter such as the SP1-32m promoter of SEQ ID No. 21, and optionally comprising sequences for integration into the genome of the host cell;
an expression plasmid encoding a pseudotyping envelope derived from an RNA virus, said expression plasmid comprising a polynucleotide encoding an envelope protein or proteins for pseudotyping, wherein said envelope pseudotyping protein is advantageously from a VSV and is in particular a VSV-G of the Indiana strain or of the New Jersey strain and,
an encapsidation plasmid, which either comprises lentiviral, especially HIV-1, gag-pol packaging sequences suitable for the production of integration-competent vector particles or modified gag-pol packaging sequences suitable for the production of integration-deficient vector particles.

The invention thus also concerns lentiviral vector particles as described above, which are the product recovered from a stable cell line transfected with:
a vector plasmid comprising (i) lentiviral, especially HIV-1, cis-active sequences necessary for packaging, reverse transcription, and transcription and further comprising a functional lentiviral, especially HIV-1, DNA flap element and optionally comprising cis-active sequences necessary for integration, said vector plasmid further comprising, (ii) a polynucleotide of a codon-optimized sequence for murine or for human of the gene encoding the fusion polypeptide of the invention, comprising one or more antigenic polypeptide(s) or immunogenic fragment(s) thereof of one or more pathogens as disclosed herein, under the control of regulatory expression sequences, especially a promoter;
a VSV-G envelope expression plasmid comprising a polynucleotide encoding a VSV-G envelope protein in particular VSV-G of the Indiana strain or of the New Jersey strain, wherein said polynucleotide is under the control of regulating expression sequences, in particular regulatory expression sequences comprising a promoter, and;
an encapsidation plasmid, wherein the encapsidation plasmid either comprises lentiviral, especially HIV-1, gag-pol coding sequences suitable for the production of integration-competent vector particles or modified gag-pol coding sequences suitable for the production of integration-deficient vector particles, wherein said gag-pol sequences are from the same lentivirus sub-family as the DNA flap element, wherein said lentiviral gag-pol or modified gag-pol sequence is under the control of regulating expression sequences.

The stable cell lines expressing the vector particles of the invention are in particular obtained by transfection of the plasmids.

The polynucleotide encodes the fusion polypeptide according to the invention, which comprises comprises a first polypeptide comprising (i) an MHC-II-associated light invariant chain (li) or (ii) the transmembrane domain of the transferrin receptor (TfR), and one or more antigenic polypeptide(s) of a pathogen, according to any embodiment disclosed in the present specification.

Accordingly, the vector plasmid may comprise one or several expression cassettes for the expression of the various antigenic polypeptides or may comprise bi-cistronic or multi-cistronic expression cassettes where the polynucleotides encoding the fusion polypeptide comprising the antigenic polypeptide(s) and optionally additional various polypeptides are separated by an IRES sequence of viral origin (Internal Ribosome Entry Site), or it may encode fusion protein(s).

The internal promoter contained in the vector genome and controlling the expression of the polynucleotide encoding an antigenic polypeptide of the pathogen (as a transgene or in an expression cassette) may be selected from the promoters of the following genes: MHC Class I promoters, such as human β2-microglobulin promoter (β2M promoter), the SP1-β2m promoter, or EF1α, human PGK, PPI (preproinsulin), thiodextrin, HLA DR invariant chain (P33), HLA DR alpha chain, Ferritin L chain or Ferritin H chain, Chymosin beta 4, Chimosin beta 10, or Cystatin Ribosomal Protein L41 CMVie or chimeric promoters such as GAG (CMV early enhancer/chicken β actin) disclosed in Jones S. et al (2009) or BCUAG.

A promoter among the above-cited internal promoters may also be selected for the expression of the envelope protein(s) and packaging (gag-pol derived) proteins.

The following particular embodiments may be carried out when preparing the lentiviral vector based on human lentivirus, and especially based on HIV-1 virus.

According to the invention, the genome of the lentiviral vector is derived from a human lentivirus, especially from the HIV lentivirus. In particular, the pseudotyped lentiviral vector is an HIV-based vector, such as an HIV-1, or HIV-2 based vector, in particular is derived from HIV-1M, for example from the BRU or LAI isolates. Alternatively, the lentiviral vector providing the necessary sequences for the vector genome may be originating from lentiviruses such as EIAV, CAEV, VISNA, FIV, BIV, SIV, HIV-2, HIV-O which are capable of transducing mammalian cells.

As stated above, when considering it apart from the recombinant polynucleotide that it finally contains, the vector genome is a replacement vector in which the nucleic acid between the 2 long terminal repeats (LTRs) in the original lentivirus genome has been restricted to cis-acting sequences for DNA or RNA synthesis and processing, including for the efficient delivery of the transgene to the nuclear of cells in the host, or at least is deleted or mutated for essential nucleic acid segments that would enable the expression of lentiviral structure proteins including biological functional GAG polyprotein and possibly POL and ENV proteins.

In a particular embodiment, the 5′ LTR and 3′ LTR sequences of the lentivirus are used in the vector genome, but the 3′ LTR at least is modified with respect to the 3′ LTR of the original lentivirus at least in the U3 region which for example can be deleted or partially deleted for the enhancer (delta U3). The 5′ LTR may also be modified, especially in its promoter region where for example a Tat-independent promoter may be substituted for the U3 endogenous promoter.

In a particular embodiment the vector genome comprises one or several of the coding sequences for Vif-, Vpr, Vpu- and Nef-accessory genes (for HIV-1 lentiviral vectors). Alternatively, these sequences can be deleted independently or each other or can be non-functional (second-generation lentiviral vector).

The vector genome of the lentiviral vector particles comprises, as an inserted cis-acting fragment, at least one polynucleotide consisting in the DNA flap element or containing such DNA flap element. In a particular embodiment, the DNA flap is inserted upstream of the polynucleotide encoding the fusion polypeptide of the invention carrying the antigenic polypeptide(s) and is advantageously—although not necessarily—located in an approximate central position in the vector genome. A DNA flap suitable for the invention may be obtained from a retrovirus, especially from a lentivirus, in particular a human lentivirus especially a HIV-1 retrovirus, or from a retrovirus-like organism such as retrotransposon. It may be alternatively obtained from the CAEV (Caprine Arthritis Encephalitis Virus) virus, the EIAV (Equine Infectious Anaemia Virus) virus, the VISNA virus, the SIV (Simian Immunodeficiency Virus) virus or the FIV (Feline Immunodeficiency Virus) virus. The DNA flap may be either prepared synthetically (chemical synthesis) or by amplification of the DNA providing the DNA Flap from the appropriate source as defined above such as by Polymerase chain reaction (PCR). In a more preferred embodiment, the DNA flap is obtained from an HIV retrovirus, for example HIV-1 or HIV-2 virus including any isolate of these two types.

The DNA flap (also designated cPPT/CTS) (defined in Zennou V. et al. ref 27, 2000, Cell vol 101, 173-185 or in WO 99/55892 and WO 01/27304), is a structure which is central in the genome of some lentiviruses especially in HIV, where it gives rise to a 3-stranded DNA structure normally synthesized during especially HIV reverse transcription and which acts as a cis-determinant of HIV genome nuclear import. The DNA flap enables a central strand displacement event controlled in cis by the central polypurine tract (cPPT) and the central termination sequence (CTS) during reverse transcription. When inserted in lentiviral-derived vectors, the polynucleotide enabling the DNA flap to be produced during reverse-transcription, stimulates gene transfer efficiency and complements the level of nuclear import to wild-type levels (Zennou et al., Cell, 2000 Cell vol 101, 173-185 or in WO 99/55892 and WO 01/27304).

Sequences of DNA flaps have been disclosed in the prior art, especially in the above cited patent applications. These sequences are also disclosed in the sequence of the pTRIP vector herein described. They are preferably inserted as a fragment, optionally with additional flanking sequences, in the vector genome, in a position which is preferably near the centre of said vector genome. Alternatively, they may be inserted immediately upstream from the promoter controlling the expression of the polynucleotide(s) encoding the fusion polypeptide of the invention. Said fragments comprising the DNA flap, inserted in the vector genome may have a sequence of about 80 to about 200 bp, depending on its origin and preparation.

According to a particular embodiment, a DNA flap has a nucleotide sequence of about 90 to about 140 nucleotides.

In HIV-1, the DNA flap is a stable 99-nucleotide-long plus strand overlap. When used in the genome vector of the lentiviral vector of the invention, it may be inserted as a longer sequence, especially when it is prepared as a PCR fragment. A particular appropriate polynucleotide comprising the structure providing the DNA flap is a 124-base pair polymerase chain reaction (PCR) fragment encompassing the cPPT and CTS regions of the HIV-1 DNA.

It is specified that the DNA flap used in the genome vector and the polynucleotides of the encapsidation plasmid encoding the GAG and POL polyproteins should originate from the same lentivirus sub-family or from the same retrovirus-like organism.

Preferably, the other cis-activating sequences of the genome vector also originate from the same lentivirus or retrovirus-like organism, as the one providing the DNA flap.

The vector genome may further comprise one or several unique restriction site(s) for cloning the recombinant polynucleotide.

In a preferred embodiment, in said vector genome, the 3′ LTR sequence of the lentiviral vector genome is devoid of at least the activator (enhancer) and possibly the promoter of the U3 region. In another particular embodiment, the 3′ LTR region is devoid of the U3 region (delta U3). In this respect, reference is made to the description in WO 01/27300 and WO 01/27304.

In a particular embodiment, in the vector genome, the U3 region of the LTR 5′ is replaced by a non lentiviral U3 or by a promoter suitable to drive tat-independent primary transcription. In such a case, the vector is independent of tat transactivator (third generation vector).

The vector genome also comprises the psi (w) packaging signal. The packaging signal is derived from the N-terminal fragment of the gag ORF. In a particular embodiment, its sequence could be modified by frameshift mutation(s) in order to prevent any interference of a possible transcription/translation of gag peptide, with that of the transgene.

The vector genome may optionally also comprise elements selected among a splice donor site (SD), a splice acceptor site (SA) and/or a Rev-responsive element (RRE).

According to a particular embodiment, the vector plasmid (or added genome vector) comprises the following cis-acting sequences for a transgenic expression cassette:
1. The LTR sequence (Long-Terminal Repeat), required for reverse transcription, the sequences required for transcription and including optionally sequences for viral DNA integration. The 3′ LTR is deleted in the U3 region at least for the promoter to provide SIN vectors (Self-inactivating), without perturbing the functions necessary for gene transfer, for two major reasons: first, to avoid trans-activation of a host gene, once the DNA is integrated in the genome and secondly to allow self-inactivation of the viral cis-sequences after retrotranscription. Optionally, the tat-dependent U3 sequence from the 5′-LTR which drives transcription of the genome is replaced by a non endogenous promoter sequence. Thus, in target cells only sequences from the internal promoter will be transcribed (transgene).
2. The ψ region, necessary for viral RNA encapsidation.
3. The RRE sequence (REV Responsive Element) allowing export of viral messenger RNA from the nucleus to the cytosol after binding of the Rev protein.
4. The DNA flap element (cPPT/CTS) to facilitate nuclear import.
5. Optionally post-transcriptional regulatory elements, especially elements that improve the expression of fusion polypeptide and/or antigenic polypeptide in dendritic cells, such as the WPRE cis-active sequence (Woodchuck hepatitis B virus Post-Responsive Element) also added to optimize stability of mRNA (Zufferey et al., 1999), the matrix or scaffold attachment regions (SAR and MAR sequences) such as those of the immunoglobulin-kappa gene (Park F. et al Mol Ther 2001; 4:164-173). 20

The lentiviral vector of the invention is non replicative (replication-incompetent) i.e., the vector and lentiviral vector genome are regarded as suitable to alleviate concerns regarding replication competent lentiviruses and especially are not able to form new particles budding from the infected host cell after administration. This may be achieved in well-known ways as the result of the absence in the lentiviral genome of the gag, pol or env genes, or their absence as “functional genes”. The gag and pol genes are thus, only provided in trans. This can also be achieved by deleting other viral coding sequence(s) and/or cis-acting genetic elements needed for particles formation.

By “functional” it is meant a gene that is correctly transcribed, and/or correctly expressed. Thus, if present in the lentiviral vector genome of the invention in this embodiment contains sequences of the gag, pol, or env are individually either not transcribed or incompletely transcribed; the expression “incompletely transcribed” refers to the alteration in the transcripts gag, gag-pro or gag-pro-pol, one of these or several of these being not transcribed. Other sequences involved in lentiviral replication may also be mutated in the vector genome, in order to achieve this status. The absence of replication of the lentiviral vector should be distinguished from the replication of the lentiviral genome. Indeed, as described before, the lentiviral genome may contain an origin of replication ensuring the replication of the lentiviral vector genome without ensuring necessarily the replication of the vector particles.

In order to obtain lentiviral vectors according to the invention, the vector genome (as a vector plasmid) must be encapsidated in particles or pseudo-particles. Accordingly, lentiviral proteins, except the envelope proteins, have to be provided in trans to the vector genome in the producing system, especially in producing cells, together with the vector genome, having recourse to at least one encapsidation plasmid carrying the gag gene and either the pol lentiviral gene or an integrative-incompetent pol gene, and preferably lacking some or all of the coding sequences for Vif-, Vpr, Vpu- and Nef-accessory genes and optionally lacking Tat (for HIV-1 lentiviral vectors).

A further plasmid is used, which carries a polynucleotide encoding the envelope pseudotyping protein(s) selected for pseudotyping lentiviral vector particles.

In a preferred embodiment, the packaging plasmid encodes only the lentiviral proteins essential for viral particle synthesis. Accessory genes whose presence in the plasmid could raise safety concerns are accordingly removed. Accordingly, viral proteins brought in trans for packaging are respectively as illustrated for those originating from HIV-1:
1. GAG proteins for building of the matrix (MA, with apparent Molecular Weight p17), the capsid (CA, p24) and nucleocapsid (NC, p6).
2. POL encoded enzymes: integrase, protease and reverse transcriptase.
3. TAT and REV regulatory proteins, when TAT is necessary for the initiation of LTR-mediated transcription; TAT expression may be omitted if the U3 region of 5′LTR is substituted for a promoter driving tat-independent transcription. REV may be modified and accordingly used for example in a recombinant protein which would enable recognition of a domain replacing the RRE sequence in the vector genome, or used as a fragment enabling binding to the RRE sequence through its RBD (RNA Binding Domain).

In order to avoid any packaging of the mRNA generated from the genes contained in the packaging plasmid in the viral particles, the w region is removed from the packaging plasmid. A heterologous promoter is inserted in the plasmid to avoid recombination issues and a poly-A tail is added 3′ from the sequences encoding the proteins. Appropriate promoters have been disclosed above.

The envelope plasmid encodes the envelope protein(s) for pseudotyping which are disclosed herein, under the control of an internal promoter, as disclosed herein.

Any or all the described plasmids for the preparation of the lentiviral vector particles of the invention may be codon optimized (CO) in the segment encoding proteins. Codon optimization according to the invention is preferably performed to improve translation of the coding sequences contained in the plasmids, in mammalian cells, murine or especially human cells. According to the invention, codon optimization is especially suited to directly or indirectly improve the preparation of the vector particles or to improve their uptake by the cells of the host to whom they are administered, or to improve the efficiency of the transfer of the polynucleotide encoding the fusion polypeptide comprising the antigenic polypeptide (transgene) in the genome of the transduced cells of the host. Methods for optimizing codons are well known in the art and codon optimization is especially performed using available programs to that effect. Codon optimization is illustrated for the coding sequences used in the examples.

In a particular embodiment of the invention, the pseudotyped lentiviral vector is also, or alternatively, integrative-competent, thus enabling the integration of the vector genome and of the recombinant polynucleotide which it contains into the genome of the transduced cells or in the cells of the host to whom it has been administered.

In another particular embodiment of the invention, the pseudotyped lentiviral vector is also, or alternatively, integrative-incompetent. In such a case, the vector genome and thus the recombinant polynucleotide which it contains do not integrate into the genome of the transduced cells or in the cells of the host to whom it has been administered.

The recombinant lentiviral vector particle of the invention may thus be a recombinant integration-deficient lentiviral vector particle, in particular wherein the recombinant integration-deficient lentiviral vector particle is a HIV-1 based vector particle and is integrase deficient as a result of a mutation of the integrase gene encoded in the genome of the lentivirus in such a way that the integrase is not expressed or not functionally expressed, in particular the mutation in the integrase gene leads to the expression of an integrase substituted on its amino acid residue 64, in particular the substitution is D64V in the catalytic domain of the HIV-1 integrase encoded by Pol.

The present invention relates to the use of a lentiviral vector wherein the expressed integrase protein is defective and which further comprises a polynucleotide especially encoding the fusion polypeptide of the invention, in particular comprising at least one antigenic polypeptide carrying epitope(s) of a pathogen, in an immunogenic composition.

By “integration-incompetent”, it is meant that the integrase, preferably of lentiviral origin, is devoid of the capacity of integration of the lentiviral genome into the genome of the host cells i.e., an integrase protein mutated to specifically alter its integrase activity.

Integration-incompetent lentiviral vectors are obtained by modifying the pol gene encoding the Integrase, resulting in a mutated po/gene encoding an integrative deficient integrase, said modified pol gene being contained in the encapsidation plasmid. Such integration-incompetent lentiviral vectors have been described in patent application WO 2006/010834. Accordingly, the integrase capacity of the protein is altered whereas the correct expression from the encapsidation plasmid of the GAG, PRO and POL proteins and/or the formation of the capsid and hence of the vector particles, as well as other steps of the viral cycle, preceding or subsequent to the integration step, such as the reverse transcription, the nuclear import, stay intact. An integrase is said defective when the integration that it should enable is altered in a way that an integration step takes place less than 1 over 1000, preferably less than 1 over 10000, when compared to a lentiviral vector containing a corresponding wild-type integrase.

In a particular embodiment of the invention, the defective integrase results from a mutation of class 1, preferably amino acid substitutions (one-amino acid substitution) or short deletions fulfilling the requirements of the expression of a defective integrase. The mutation is carried out within the po/gene. These vectors may carry a defective integrase with the mutation D64V in the catalytic domain of the enzyme, which specifically blocks the DNA cleaving and joining reactions of the integration step. The D64V mutation decreases integration of pseudotyped HIV-1 up to 1/10,000 of wild type, but keep their ability to transduce non dividing cells, allowing efficient transgene expression.

Other mutations in the pol gene which are suitable to affect the integrase capacity of the integrase of HIV-1 are the following: H12N, H12C, H16C, H16V, S81 R, D41A, K42A, H51A, Q53C, D55V, D64E, D64V, E69A, K71A, E85A, E87A, D116N, D1161, D116A, N120G, N1201, N120E, E152G, E152A, D-35-E, K156E, K156A, E157A, K159E, K159A, K160A, R166A, D167A, E170A, H171A, K173A, K186Q, K186T, K188T, E198A, R199C, R199T, R199A, D202A, K211A, Q214L, Q216L, Q221 L, W235F, W235E, K236S, K236A, K246A, G247W, D253A, R262A, R263A and K264H.

In a particular embodiment, mutation in the pol gene is performed at either of the following positions D64, D116 or E152, or at several of these positions which are in the catalytic site of the protein. Any substitution at these positions is suitable, including those described above.

Another proposed substitution is the replacement of the amino acid residues RRK (positions 262 to 264) by the amino acid residues AAH.

In a particular embodiment of the invention, when the lentiviral vector is integration-incompetent, the lentiviral genome further comprises an origin of replication (ori), whose sequence is dependent on the nature of cells where the lentiviral genome has to be expressed. Said origin of replication may be from eukaryotic origin, preferably of mammalian origin, most preferably of human origin. It may alternatively be of viral origin, especially coming from circular episomic DNA, as in SV40 or RPS. It is an advantageous embodiment of the invention to have an origin or replication inserted in the lentiviral genome of the lentiviral vector of the invention. Indeed, when the lentiviral genome does not integrate into the cell host genome (because of the defective integrase), the lentiviral genome is lost in cells that undergo frequent cell divisions; this is particularly the case in immune cells, such as B or T cells. The presence of an origin of replication ensures that at least one lentiviral genome is present in each cell, even after cell division, accordingly maximizing the efficiency of the immune response.

The lentiviral vector genome of said lentiviral vectors of the invention may especially be derived from HIV-1 plasmid pTRIPAU3.CMV-GFP deposited at the CNCM (Institut Pasteur, 25-28, rue du Docteur Roux, 75724 Paris Cedex 15, France) on Oct. 11, 1999 under number I-2330 (also described in WO01/27300) or variants thereof. The lentiviral vector genome of said lentiviral vectors of the invention may especially be derived from HIV-1 plasmid pFlap-SP1beta2m-GFP-WPREm deposited at the CNCM (Institut Pasteur, 25-28, rue du Docteur Roux, 75724 Paris Cedex 15, France) on Feb. 16, 2021 under number CNCM I-5657 or variants thereof.

In one embodiment, the lentiviral vector genome is derived from the plasmid having the sequence of SEQ ID No. 20, SEQ ID No. 25 or SEQ ID No. 26. In particular, the lentiviral vector genome comprises a sequence having at least 70%, in particular 80% or 90%, more particularly 95% or 99% sequence identity with SEQ ID No. 20, SEQ ID No. 25 or SEQ ID No. 26.

When the vector genome is derived from these particular plasmids, a sequence of a recombinant polynucleotide encoding the fusion polypeptide of the invention, in particular comprising an antigenic polypeptide of a pathogen as disclosed in the present application, is inserted therein, in addition or in replacement of the GFP coding fragment in SEQ ID No. 20, the li-EsxH fragment of SEQ ID No. 25 or the TfR-EsxH fragment of SEQ ID No. 26. The promoter, i.e. CMV or SP1-2m promoters may also be substituted by another promoter, especially one of the promoters disclosed above, especially in relation to the expression of the transgene.

The WPRE or WPREm sequences also contained in the particular pFlap (pFLAPDeltaU3) and pTRIP vectors may optionally be deleted.

Vector particles may be produced after transfection of appropriate cells (such as mammalian cells or human cells, such as Human Embryonic Kidney cells illustrated by 293 T cells) by said plasmids, or by other processes. In the cells used for the expression of the lentiviral particles, all or some of the plasmids may be used to stably express their coding polynucleotides, or to transiently or semi-stably express their coding polynucleotides.

The concentration of particles produced can be determined by measuring the P24 (capsid protein for HIV-1) content of cell supernatants.

The lentiviral vector of the invention, once administered into the host, infects cells of the host, possibly specific cells, depending on the envelope proteins it was pseudotyped with. The infection leads to the release of the lentiviral vector genome into the cytoplasm of the host cell where the retro-transcription takes place. Once under a triplex form (via the DNA flap), the lentiviral vector genome is imported into the nucleus, where the polynucleotide(s) encoding polypeptide(s) of antigen(s) of the pathogen is (are) expressed via the cellular machinery. When non-dividing cells are transduced (such as DC), the expression may be stable. When dividing cells are transduced, such as B cells, the expression is temporary in absence of origin of replication in the lentiviral genome, because of nucleic acid dilution and cell division. The expression may be longer by providing an origin of replication ensuring a proper diffusion of the lentiviral vector genome into daughter cells after cell division. The stability and/or expression may also be increased by insertion of MAR (Matrix Associated Region) or SAR (Scaffold Associated Region) elements in the vector genome.

Indeed, these SAR or MAR regions are AT-rich sequences and enable to anchor the lentiviral genome to the matrix of the cell chromosome, thus regulating the transcription of the polynucleotide encoding the fusion polypeptide of the invention comprising at least one antigenic polypeptide, and particularly stimulating gene expression of the transgene and improving chromatin accessibility.

If the lentiviral genome is non integrative, it does not integrate into the host cell genome. Nevertheless, the at least one polypeptide encoded by the transgene is sufficiently expressed and longer enough to be processed, associated with MHC molecules and finally directed towards the cell surface. Depending on the nature of the polynucleotide(s) encoding antigenic polypeptide(s) of a pathogen, the at least one polypeptide epitope associated with the MHC molecule triggers a cellular immune response.

Unless otherwise stated, or unless technically not relevant, the characteristics disclosed in the present application with respect to any of the various features, embodiments or examples of the structure or use of the lentiviral particles, especially regarding their envelope protein(s), or the recombinant polynucleotide, may be combined according to any possible combinations.

The invention further relates to a combination of compounds for separate administration to a mammalian host, which comprises at least:
(i) lentiviral vector particles of the invention which are pseudotyped with a first determined heterologous viral envelope pseudotyping protein or viral envelope pseudotyping proteins; such first pseudotyping protein may be from the New-Jersey strain of VSV;
(ii) provided separately from lentiviral vector particles in (i), lentiviral vector particles of the invention which are pseudotyped with a second determined heterologous viral envelope pseudotyping protein or viral envelope pseudotyping proteins distinct from said first heterologous viral envelope pseudotyping protein(s); such second pseudotyping protein may be from the Indiana strain of VSV.

In another embodiment of the invention, possibly in combination with the above disclosed alternative forms of the nucleic acid, the polynucleotide encoding the fusion polypeptide of the invention, comprising at least one antigenic polypeptide is structurally modified and/or chemically modified. Illustrative thereof a polynucleotide comprises a Kozak consensus sequence in its 5′ region. Other nucleic acid sequences that are not of lentiviral origin may be present in the vector genome are IRES sequence(s) (Internal Ribosome entry site) suitable to initiate polypeptide synthesis,

WPRE sequence or modified WPRE sequence as post-transcriptional regulatory element to stabilize the produced RNA.

According to another embodiment of the invention, if multiple heterologous polypeptides are encoded by one vector genome, the coding sequences may optionally be separated by a linker moiety which is either a nucleic acid-based molecule or a non-nucleic acid-based molecule. Such a molecule may be a functionalized linker molecule aimed at recognizing a 3′ functionalized nucleic acid to which it shall be linked. A sequence suitable to function as a linker may alternatively be a nucleic acid which encodes a self-cleaving peptide, such as a 2A peptide.

Further features and properties of the present invention, including features to be used in the embodiments described above will be described in the examples and figures which follow and may accordingly be used to characterise the invention.

### SEQUENCE LISTING

SEQ ID No. 1: li-HAEP amino acid sequence
SEQ ID No. 2: li-HAEP DNA sequence
SEQ ID No. 3: li-HAEPA amino acid sequence
SEQ ID No. 4: li-HAEPA DNA sequence
SEQ ID No. 5: li-EsxH amino acid sequence
SEQ ID No. 6: li-EsxH DNA sequence
SEQ ID No. 7: TfR1-118-EsxH amino acid sequence
SEQ ID No. 8: TfR1-118-EsxH DNA sequence
SEQ ID No. 9: SP-EsxH-MITD amino acid sequence
SEQ ID No. 10: SP-EsxH-MITD DNA sequence
SEQ ID No. 11: human invariant chain (li) amino acid sequence
SEQ ID No. 12: human invariant chain (li) DNA sequence
SEQ ID No. 13: transmembrane domain of the human transferrin receptor, amino acid sequence
SEQ ID No. 14: transmembrane domain of the human transferrin receptor, DNA sequence
SEQ ID No. 15: EsxH₂₀₋₂₈ epitope amino acid sequence
SEQ ID No. 16: EsxH₇₄₋₈₈ epitope amino acid sequence
SEQ ID No. 17: EsxA₁₋₂₀ epitope amino acid sequence
SEQ ID No. 18: PE-19₁₋₁₈ epitope amino acid sequence
SEQ ID No. 19: Ag85A₂₄₁₋₂₆₀ epitope amino acid sequence
SEQ ID No. 20: plasmid pFlap-SP1beta2m-GFP-WPREm (SP1beta2m promoter, GFP transgene and WPREm) DNA sequence
SEQ ID No. 21: SP1-human B2-microglobulin promoter
SEQ ID No. 22: BCUAG promoter
SEQ ID No. 23: mutant WPRE
SEQ ID No. 24: humanized li-antigen amino acid sequence (human li-EsxH)
SEQ ID No. 25: recombinant pFLAP with fusion sequence of humanized li-EsxH antigen nucleotide sequence (β2-microglobulin Promoter)
SEQ ID No. 26: recombinant pFLAP with fusion sequence of humanized TfR-EsxH antigen nucleotide sequence (β2-microglobulin Promoter)
SEQ ID No. 27: GGGD linker
SEQ ID No. 28: NNGG linker
SEQ ID No. 29: NNDD linker
SEQ ID No. 30: Nucleotide sequence of Green Fluorescent Protein (GFP) gene (codon optimized)
SEQ ID No. 31: Amino-acid sequence of Green Fluorescent Protein (GFP) gene

### LEGENDS OF THE FIGURES

FIG. 1. Intra-phagocyte quantitation of Ag85A/B and EsxA secretion by Beijing or non-Beijing Mtb clinical isolates. (A-B) Bone-marrow-derived DC (H-2^{b}) were infected with various CFU/ml of each Mtb strain from a set of non-Beijing or Beijing clinical isolates, numbered as indicated in Table S1. After overnight incubation, MHC-II-restricted T-cell hybridomas specific to Ag85A/B (DE10) (A) or EsxA (NB11) (β) were added and the concentration of IL-2 produced by T-cell hybridomas, proportional to the amounts of Mtb antigens secreted inside the DC phagosomes, was determined by ELISA, after 24h incubation. (C) Intra-phagocyte amounts of Ag85A/B or EsxA secretion, as determined in DC infected with 4×10³ CFU/ml.

FIG. 2. Tailoring LV to direct antigens towards MHC-II processing pathway. (A) Failure of CD4⁺ T-cell induction by conventional LV. Cytometric analysis of splenocytes from C57BL/6 mice immunized with a conventional LV encoding for the fusion of EsxA-Ag85A-EspC-EsxH-PE19 Mtb immunogens. Shown are CD4⁺ and CD8⁺ T-splenocyte IFN-γ responses subsequent to in vitro stimulation with EspC: 45-54 peptide, which contains both MHC-I and -II-restricted epitopes in H-2^{b}, or a negative control peptide. (β) Scheme of full length EsxH protein, with sequences potentially facilitating its routing through the MHC-II pathway added at its N- or C-ter extremity. (C) Presentation of MHC-I- or -II-restricted EsxH epitopes by DC (H-2^{d}), transduced with 1×10⁶ TU/ml of LV encoding EsxH alone, li-EsxH, TfR-EsxH or SP-EsxH-MITD, and co-cultured at day 3 post-transduction with T-cell hybridomas specific to EsxH: 20-28 and restricted by K^{d} (YB8) (top) or specific to EsxH: 74-88 and restricted by I-Ad (1H2) (bottom). Results are Mean±SD of concentrations of IL-2, produced by T-cell hybridomas after overnight co-culture.

FIG. 3. Induction of systemic or mucosal CD4⁺ and CD8⁺ T-cell responses by the optimized LV. BALB/c (H-2^{d}) mice (n=3/group) were immunized s.c. with 5×10⁷ TU of LV::li-EsxH alone (1) or adjuvanted with polyI:C (2) or cGAMP (3). At 11 dpi, EsxH-specific Th1 cytokine responses of splenocytes were analyzed by ICS in individual mice. (A) Gating strategy carried out on cytokine producing CD4⁺ or CD8⁺ T cells. (β-C) Recapitulative frequencies of each (multi) functional population within the CD4⁺ (β) or CD8⁺ (C) T subset. (D-E) BALB/c mice (n=3/group) were immunized i.n. with 5×10⁷ TU of LV::li-EsxH alone or adjuvanted with polyI:C or cGAMP. At 13 dpi, EsxH-specific lung CD4⁺ or CD8⁺ T-cell responses were analyzed by co-culture of lymphocytes enriched from the lungs with homologous DC loaded with EsxH: 74-88 (MHC-II) (D) or with EsxH: 20-28 (MHC-I) (E). IL-2, IL-17A or IFN-γ contents in the co-culture supernatants were quantitated by ELISA.

FIG. 4. Characterization of mucosal CD4⁺ or CD8⁺ T-cell responses induced by the optimized LV. BALB/c (H-2^{d}) mice (n=3/group) were immunized i.n. with 5×10⁷ TU of LV::li-EsxH alone or adjuvanted with polyI:C or cGAMP. At 13 dpi, lung CD4⁺ (A) or CD8⁺ (E) T cells were discriminated for their location inside the interstitium (CD45i.v) or in the vasculature (CD45_{i.v}⁺) by an i.v. injection of PE-anti-CD45 mAb, 3 min before sacrifice. (β, F) Profile of CD27 versus CD62L expression or (C, D, G, H) cytokine production, as detected by ICS in lung CD4⁺ or CD8⁺ T cells of the lung interstitium or vasculature. Results, representative of 2-3 independent experiments, were from lungs pooled per experimental groups to reach cell numbers high enough for accurate cytometric analyses.

FIG. 5. Characterization of mucosal innate immunity induced by LV i.n. administration. (A) Cytometric gating strategy used on total lung cells to analyze various mucosal innate immune cell populations. Shown are cells from PBS-injected negative controls. (β) Percentages of each innate immune subset versus total lung CD45⁺ cells in C57BL/6 mice injected i.n. with PBS, LV alone, or cGAMP-adjuvanted LV, as determined at 2 dpi. Results are from 3 individual mice/group. ns=not significant, as determined by One-tailed Mann Whitney test.

FIG. 6. Potential of the poly-antigenic LV::li-HAEP at inducing CD4⁺ and CD8⁺ T cells. (A) Presentation of MHC-I- or -II-restricted epitopes by H-2^{d} or H-2^{b} DC transduced with LV::li-HAEP or LV::TB as a negative control and co-cultured at day 3 post-transduction with T-cell hybridomas specific to EsxH: 20-28, restricted by K^{d} (YB8), EsxH: 74-88, restricted by I-Ad (1G1), EsxA: 1-20 (NB11) or to PE19: 1-18 (IF6), restricted by I-Ab. (β-D) C57BL/6 (H-2^{b}) mice were immunized s.c. with 5×10⁸ TU of LV::li-HAEP or injected with PBS. At 11 dpi, the antigen-specific cytokine responses of splenocytes, determined as SFU (Spot Forming Unit) by ELISPOT (β) or ICS (C-D) in individual mice. (C-D) Shown are recapitulative frequencies of each (multi) functional population within the CD4⁺ (C) or CD8⁺ (D) T subsets, after removing the background signal observed with an irrelevant negative control peptide for each mouse.

FIG. 7. Features of mucosal T-cell responses induced by LV::li-HAEP. C57BL/6 (H-2^{b}) mice were immunized i.n. with 5×10⁸ TU of LV::li-HAEP, adjuvanted with cGAMP (n=7) or instilled with PBS (n=3). At 13 dpi, following an i.v. injection of PE-anti-CD45 mAb, 3 min before sacrifice, CD4⁺ (A) or CD8⁺ (β) lung T-cell responses were analyzed by ICS after co-culture with homologous DC loaded with EsxA: 1-20 (MHC-II), PE19: 1-18 (MHC-II), EspC: 40-54 (MHC-I, and -II), EsxH: 20-28 (MHC-I) or an irrelevant negative control peptide. Shown are recapitulative absolute numbers of each (multi) functional population within the CD4⁺ (A) or CD8⁺ (β) T subsets located inside the interstitium (CD45i.v⁻) or in the vasculature (CD45i.v⁺). (C, D) Phenotyping of interstitial (CD45_{i.v}⁻) CD4⁺ (C) or CD8⁺ (D). Results were generated with cells pooled from the lungs per group to reach enough number for cytometric analyses.

FIG. 8. Protective potential of an optimized poly-antigenic LV as a booster in TB vaccination. (A) MHC-II-restricted presentation of Ag85A, in parallel to EsxA, as detected on DC (H-2^{b}) transduced with LV::li-HAEPA or LV::TB as a negative control and co-cultured at day 3 post-transduction with Ag85A- or EsxA-specific T-cell hybridomas harboring the gene encoding ZsGreen reporter under the control of IL-2 promoter. (β) Timeline of prime with BCG::ESX-1^{Mmar}, boost with cGAMP-adjuvanted LV::li-HAEPA and challenge with Mtb H37Rv strain, performed in C57BL/6 mice (n=5-9/group). (C) Mycobacterial loads determined by CFU counting in the lungs and spleen of individual mice at week 5 post challenge. ns=not significant, * (p=0.0415), ** (p=0.0040) *** (p=0.00105), statistically significant, as determined by One-tailed Mann Whitney test. (D) Representative lung hematoxylin and eosin histopathological results. Analysis was performed on the left lung lobes of unvaccinated (left), BCG: ESX-1^{Mmar}-vaccinated (middle) or BCG::ESX-1^{Mmar}-primed and cGAMP-adjuvanted LV::li-HAEPA-boosted (right) C57BL/6 mice, at week 5 post Mtb challenge.

FIG. 9. Weak capacity of LV at inducing DC maturation. Bone-marrow-derived DC from C57BL/6 mice were left untreated (negative control), treated with Mtb at MOI=3 (positive control), or LV at the high MOI of 50. (A-C) Maturation of CD11c⁺CD11b⁺ cells (A), as monitored by flow cytometry after overnight incubation for the expression of CD40, CD80, CD86, MHC-I and MHC-II surface molecules (β). (C) MFI or percentage of bright (hi) cells. Results are representative of two independent experiments.

FIG. 10. Non-dependence of LV-mediated CD8⁺ T-cell induction on IFNAR signaling in DC. (A) Verification of the IFNAR1 deficiency in DC of the KO mice by assessing the IFNAR1 surface expression by bone-marrow DC derived from hematopoietic stem cells of ifnar^{flox/flox} pCD11c-Cre⁻ (WT) or ifnar^{flox/flox} pCD11c-Cre⁺ (KO) mice. (β-G) Mice, either ifnar^{flox/flox} pCD11c-Cre⁻ (WT) or ifnar^{flox/flox} pCD11c-Cre⁺ (KO), were immunized i.m. with 5×10⁷ TU of LV::OVA (β-C) or LV::li-EsxH (D-G). At 11 dpi, antigen-specific CD8⁺ T splenocytes were assessed through tetramer staining, ELISPOT or ICS analysis. (β) Percentages of cells stained positively with “OVA tetramer”, as compared to total CD3+CD8⁺ T splenocytes. (C) Numbers of splenocytes secreting IFN-γ after ex vivo stimulation with OVA: 257-264, as detected by ELISPOT. (D) Numbers of splenocytes secreting IFN-γ or TNF-α, after ex vivo stimulation with EsxH: 3-11 or a negative ctrl peptide or, as detected by ELISPOT (SFU=Spot Forming Unit). (E) Degranulation activity of the IFN-γ-producing CD8⁺ T cells, as evaluated by the surface CD107a staining. (F) Gating strategy used in ICS analysis performed on CD8⁺ T splenocytes. (G) Recapitulative frequencies of various (poly) functional EsxH:3-11-specific CD8⁺ T-cell effectors.

FIG. 11. Non-dependence of LV-mediated CD4⁺ T-cell induction on IFNAR Signaling in DC. Ifnar^{flox/flox} pCD11c-Cre⁻ (WT) and ifnar^{flox/flox} pCD11c-Cre⁺ (KO) mice were immunized s.c. with 5×10⁸ TU of LV::li-HAEP. At 11 dpi, antigen-specific CD4⁺ T-cell responses were assessed through ICS. Shown are recapitulative frequencies of (poly) functional CD4⁺ T splenocytes, specific to EsxA or PE19, as detected after stimulation with EsxA: 1-20 or PE19: 1-18 peptides.

FIG. 12. Comparison of the immunogenicity of LV::li-HAEP injected via i.m. or s.c. systemic routes. C57BL/6 mice were immunized i.m. or s.c. with 5×10⁸ TU of LV::li-HAEP. At 14 dpi, antigen-specific, IFN-γ or TNF-α T cell responses were assessed by ELISPOT.

FIG. 13. Maps of plasmids encoding EsxH variants or poly-antigenic fusion proteins. The codon-optimized cDNA sequences, encoding EsxH variants or poly-antigenic fusion proteins of vaccine interest (Table S3), were inserted under the SP1-β2m promoter in a pFLAP backbone plasmid.

FIG. 14. Map of the pFLAP backbone plasmid containing GFP. The sequence of a GFP transgene was inserted under the SP1-2m promoter, with a WPREm sequence

FIG. 15. Maps of plasmids for human immunization. The codon-optimized cDNA sequences, encoding EsxH variants, were inserted under the SP1-B2m promoter in a pFLAP backbone plasmid. Panel A: the EsxH antigen is fused with human li. Panel B: the EsxH antigen is fused with hTfR.

### EXAMPLES

### Introduction

According to the World Health Organization, Mycobacterium tuberculosis (Mtb) causes more than 8 million new cases of pulmonary tuberculosis (TB) each year, and remains one of the top ten causes of death and the first cause of mortality due to an infectious pathogen worldwide. Of the estimated 1.7 billion asymptomatic people latently infected with Mtb, 5 to 15% will evolve toward active TB. The risk of developing the disease is higher in individuals co-infected with HIV or affected by under-nutrition, diabetes, smoking or alcoholism (1). The only current TB vaccine is the widely administrated Mycobacterium bovis Bacillus Calmette-Guérin (BCG), given early after birth is particularly effective at inducing Th1-biased responses in infants. Although BCG is effective in protecting children against pulmonary and disseminated forms, it has a limited impact on adolescent and adult pulmonary TB and reactivation of latent TB and thus cannot prevent global bacillary spread (2). Therefore, there is an urgent need for new immunization strategies: (i) effective as pre-exposure vaccines, (ii) able to decrease the risk of primary Mtb infection, (iii) preventive against latent TB progression to active disease, or (iv) usable in TB immune-therapy.

Even though the immune correlates of TB protection are poorly understood, it is well established that protective immunity against the intracellular pathogen Mtb is mainly dependent on cell-mediated immunity. The contribution of appropriate innate and T cell-mediated immunity, notably IFN-γ/TNF-α-producing CD4⁺ Th1 cells, and at a lesser extent CD8⁺ T cells, is instrumental in anti-mycobacterial host defense, even not sufficient to reach full protection (3, 4). Since 1921, four billion people have been vaccinated by BCG and numerous improved live-attenuated vaccine candidates are in development (5). The immunity conferred by BCG is of variable duration, but admittedly limited to ˜10 years. Homologous boosting with live-attenuated vaccines and repeated administration of mycobacteria may cause adverse necrotic inflammation, namely the “Koch phenomenon”, characterized by strong expression of IL-6, IL-17, TNF-α and CXCL2, and massive recruitment of neutrophils (6). In this context, heterogeneous prime-boost regimen, relying on priming with improved live-attenuated vaccines followed by boosting with subunit vaccines, is an attractive approach to synergistically enhance the Mtb-specific protective immunity (7). We have previously elaborated a promising live-attenuated TB vaccine candidate based on BCG Pasteur strain stably complemented with the esx-1 genomic region of a fish pathogen phylogenetically related to Mtb, i.e., Mycobacterium marinum (BCG::ESX-1^{Mmar}) (8). Compared to BCG, this strain shows largely improved protective potential against TB in animal models, which is consistent with its known properties. In fact, this strain displays an enlarged antigenic repertoire, capacity to trigger the cGAS (cyclic GMP-AMP Synthase)/STING (STimulator of INterferon Genes)/IRF3 (Interferon Regulatory Factor 3)/IFN-I (type-I IFN) axis, and to reinforce the NLRP3 (NOD-Like Receptor family Protein 3) and the cytosolic DNA sensor, AIM-2 (Absent In Melanoma-2) inflammasome pathways, while displaying attenuated virulence (8, 9). In the murine TB model, BCG::ESX-1^{Mmar} vaccination reduces mycobacterial loads better than the parental BCG, but does not yet lead to sterilizing immunity, leaving the possibility to evaluate the protective potential of booster vaccines.

The use of recombinant viral vaccine vectors, expressing potent Mtb antigens, may result in a synergistic enhancement of anti-mycobacterial immunity in individuals primed with live-attenuated vaccine candidates. This can be achieved by increasing the frequencies of antigen-specific T cells, improving T-cell avidity, as well as enhancing the CD8⁺ T-cell responses that are not efficaciously induced by mycobacteria in general. Replication-defective, Lentiviral Vectors (LV) are powerful delivery systems and attractive immunization tools, based on their: (i) low genotoxic potential, (ii) capacity to accept up to 8 kb inserts, (iii) strong ability to transduce in vivo both replicating and non-dividing cells, (iv) persistent antigen expression, and (v) advantage of not being the target of preexisting anti-vector immunity in the human populations (10-15). However, one limitation of LV is their inability to target antigens to the Major Histocompatibility Complex class II (MHC-II) pathway to trigger CD4⁺ T cells, which are considered so far as the best correlates of protection against TB and multiple other diseases.

Here, we describe a new generation of LV, with the capacity to route immunogens toward the MHC-II machinery, created by adding of the MHC-II light invariant chain (li) at the N-ter part of the antigen(s) encoded by the vector. This approach and immunization strategies via the systemic or mucosal route, allowed proper implementation of antigen trafficking to the MHC-II machinery, thereby inducing in addition to CD8⁺ T cells, appropriate CD4⁺ T cells, that we thoroughly characterized for their phenotype, functions and pulmonary localization. We further report the significant protective potential of a selected optimized LV, encoding five potent Mtb antigens, used as a booster, administered via systemic and intranasal (i.n.) routes into BCG::ESX-1^{Mmar}-primed mice.

### Results

### Rational Selection of Mtb Immunogens

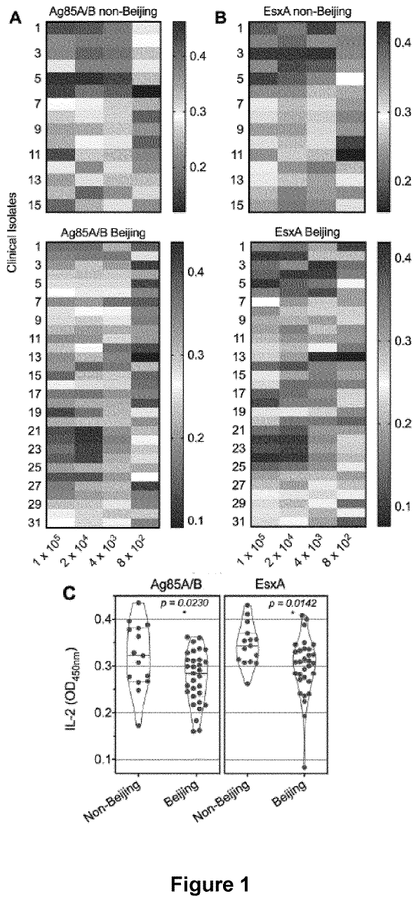

Since the T-cell targets of Mtb are predominantly secreted proteins (16, 17), to develop a poly-antigenic LV-based vaccine, we selected the following virulence-related factors: (i) EsxA (Rv3875), (ii) ESX-1 secretion-associated proteins (Esp) C (Rv3615c), both secreted via ESX-1 Type VII Secretion System (T7SS), (iii) EsxH (Rv0288, TB10.4), secreted via ESX-3 T7SS, (iv) PE19 (Rv1791), secreted via ESX-5 T7SS (18-21), and (v) Ag85A (Rv3804c) from the mycolyl transferase Ag85 complex, secreted via the Tat system (17, 22). Concerning the latter, we had previously observed that some Beijing clinical isolates expressed only minute amounts of Ag85A/B, which called into question the pertinence of the inclusion of these antigens in vaccine candidates (23). Here, to re-evaluate this assumption, we comparatively quantitated the intra-phagocyte secretion of Ag85A/B (FIG. 1A) versus EsxA (FIG. 1B) inside Dendritic Cells (DC) infected with each of the 15 non-Beijing or 31 Beijing clinical Mtb isolates listed in Table S1. This was performed using T-cell hybridomas specific to Ag85A/B or EsxA (Table S2), to measure the MHC-II-mediated presentation of T-cell epitopes derived from these antigens, which is proportional to their intra-phagocyte secretion (23). Although the average of Ag85A/B expression of the Beijing clinical isolates was statistically lower than that of the non-Beijing strains, the expression of these antigens was quite variable and many Beijing isolates were found to produce large amounts of Ag85A/B (FIG. 1C). This observation determined that Ag85A/B is in fact a relevant vaccine target.

### LV Optimization to Route Mtb Immunogens to MHC-II Pathway

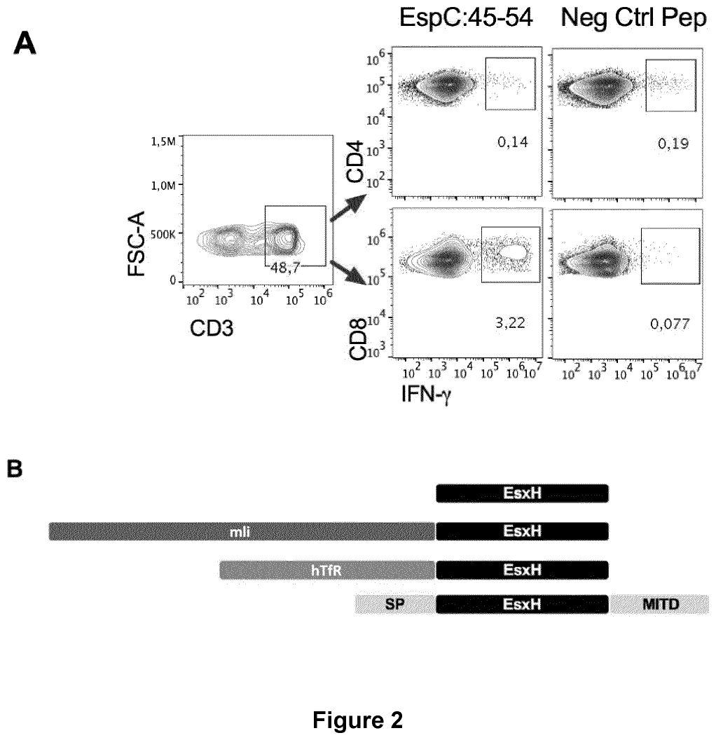

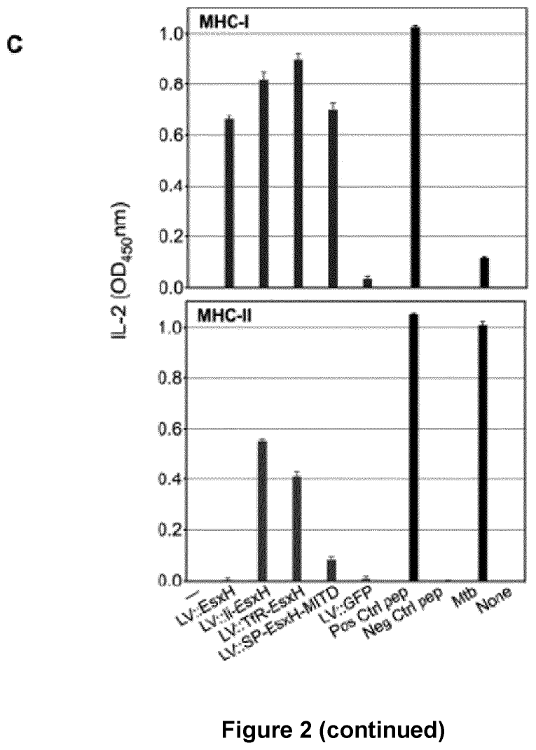

Highly efficient at routing endogenously produced antigens to MHC-I, viral vectors are however poorly effective, if not inoperative, in antigen delivery to MHC-II machinery. This was confirmed by the fact that our initial conventional LV, encoding individual EsxA, EspC, EsxH, PE19 or Ag85A, or various fusions of them, did not trigger CD4⁺ T-cell responses in mice. This was exemplified by the absence of EspC-specific CD4⁺ T cells, despite the presence of EspC-specific CD8⁺ T cells in C57BL/6 mice immunized with a conventional LV encoding a fusion of Mtb antigens including EsxA, Ag85A, EspC, EsxH, and PE19 (LV::TB) (FIG. 2A). It is noteworthy that, in addition to the MHC-II-restricted epitope within EspC:45-54 (23), this segment contains an MHC-I-restricted epitope, so far uncovered in H-2^{b} mice, and evidenced here by use of LV. To overcome LV inability to induce inducing CD4⁺ T cells, we sought to optimize this vector, as detailed below and using EsxH as reporter antigen, considering the availability of EsxH-specific, MHC-I- or -II-restricted T-cell hybridomas (Table S2) (24, 25).

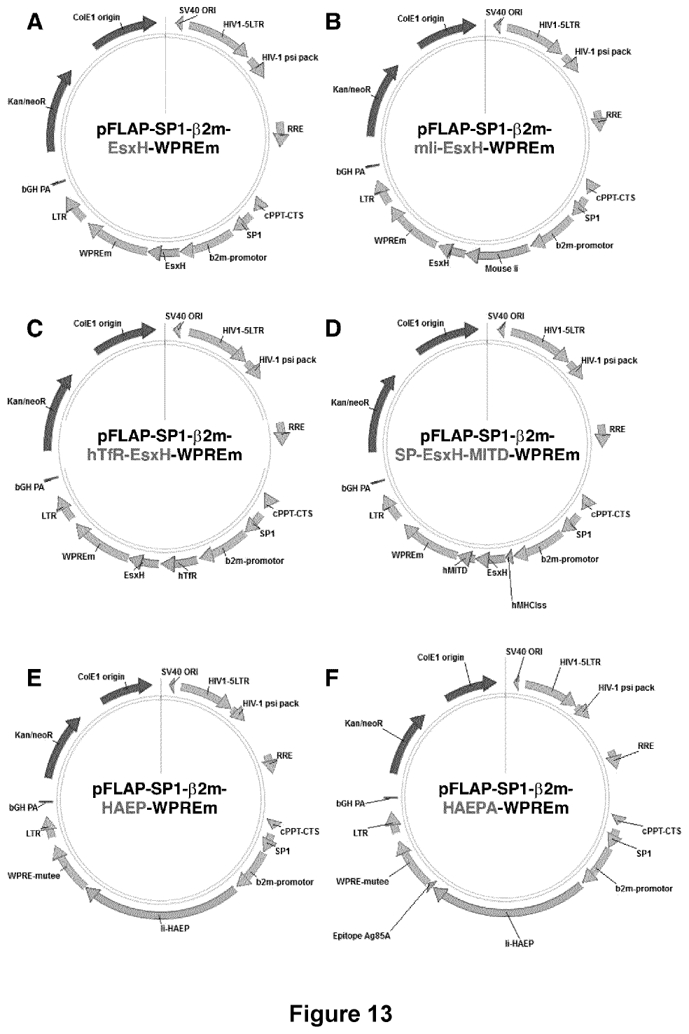

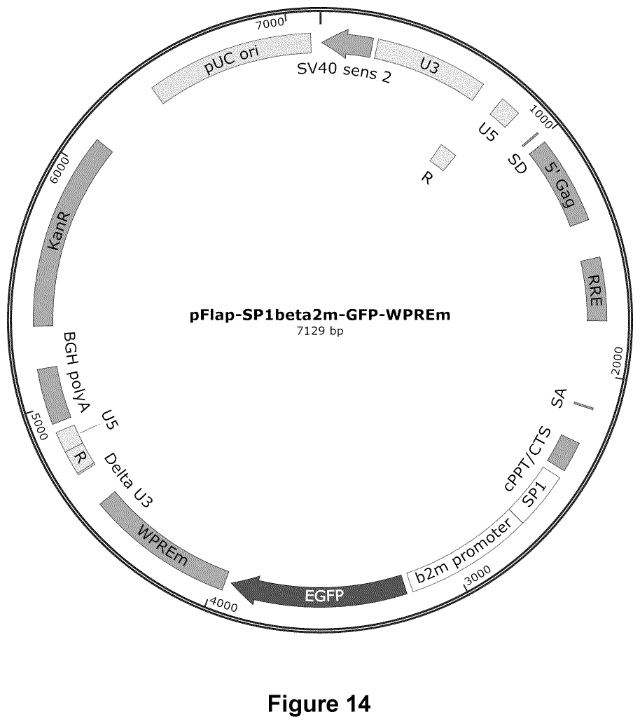

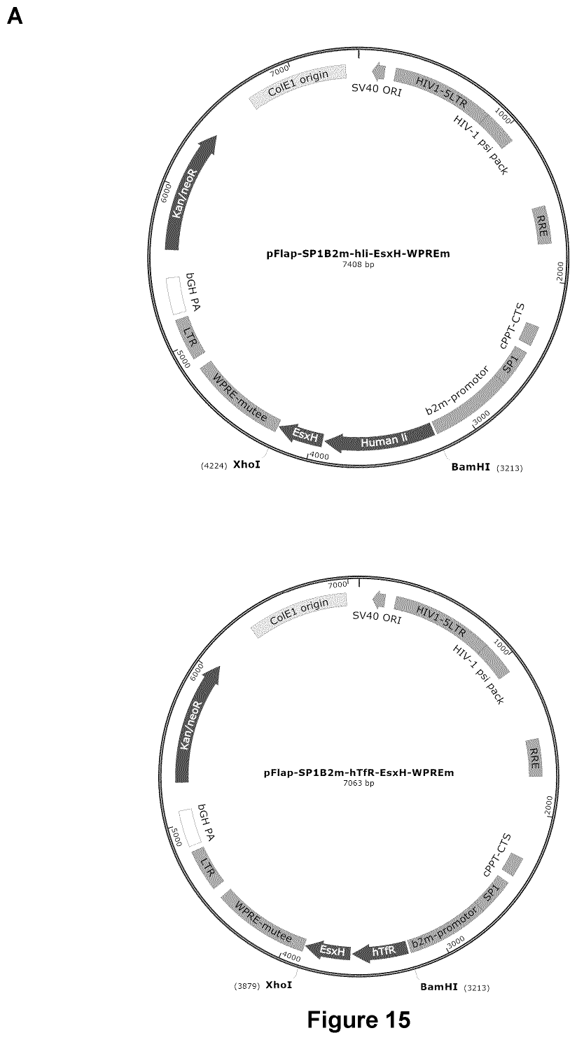

We generated a series of LV encoding: (i) EsxH alone (LV::EsxH), (ii) EsxH added at its N-ter part with the murine MHC-II “li” light invariant chain (LV::li-EsxH), to target the translated antigen to the MHC-II compartment (26, 27), (iii) EsxH added at its N-ter part with the 1-118 transmembrane domain of the human Transferrin Receptor (LV::TfR₁₋₁₁₈-EsxH), to generate a membrane-bound protein which should traffic through endosomes, to potentially gain access to the MHC-II pathway (26, 27), or (iv) EsxH added at its N-ter and C-ter ends, respectively with HLA-B-derived leader SP peptide and MHC-I Trafficking signal (LV::SP-EsxH-MITD), since the MHC-I molecules also traffic via endosomes (28) (FIG. 2B, FIG. 13). DC transduced with LV encoding either EsxH variants, were able to present efficiently the MHC-I-restricted EsxH: 20-28 epitope to specific T-cell hybridoma (FIG. 2C). In net contrast to the conventional LV::EsxH, only the optimized LV::li-EsxH, and to a lesser extent, LV::TfR₁₋₁₁₈-EsxH, were able to induce efficient MHC-II-restricted EsxH: 74-88 epitope presentation to specific T-cell hybridoma

(FIG. 2C). LV::SP-EsxH-MITD did not enable antigen presentation by MHC-II. For further experiments described below, we thus selected the li flanking strategy, which resulted in the highest presentation level via MHC-II, without impacting the presentation via MHC-I. In sum, we elaborated a new generation of LV which gains the instrumental property to provide appropriate antigen presentation, i.e., “signal 1” (29), not only via MHC-I, but also through MHC-II pathway.

Very Minor Impact of LV on Innate Immunity Versus their Noticeable T-Cell Immunogenicity

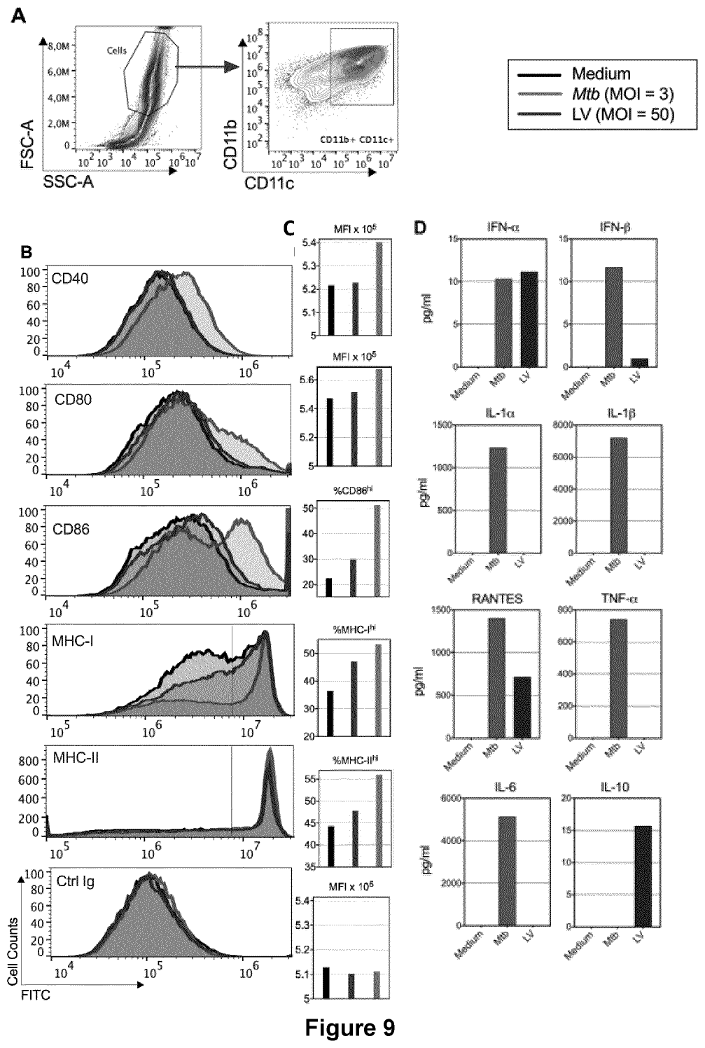

Before exploring the potential of the optimized LV at inducing CD4⁺ T cells in vivo, we assessed some properties of LV regarding induction of DC maturation or inflammatory cytokine signaling, i.e., “signal 2-3” (29). A preparation of LV, not characterized for endotoxin content, has been described to induce a few inflammatory responses in vivo (30). Another study reported some degrees of LV-induced DC maturation in vitro, attributed to the Vesicular Stomatitis Virus (VSV) G envelop glycoprotein, with which the LV are pseudo-typed (31). Murine DC, even when confronted to high amounts (MOI of 50) of our pre-GMP quality, VSV-G-pseudo-typed LV, displayed very slight phenotypic maturation, as judged by only a very minor CD86 upregulation and minute increases in the percentages of MHC-I^{hi} or -II^{hi} cells (FIG. 9A-B, C In terms of functional maturation, DC transduced with LV secreted readily detectable amounts of IFN-α, CCL5 and IL-10 and very mild amounts of IFN-β. Importantly, no IL-1α, IL-1β, IL-6 or TNF-α were detected, indicating a poor inflammatory and even anti-inflammatory properties of LV (FIG. 9 D).

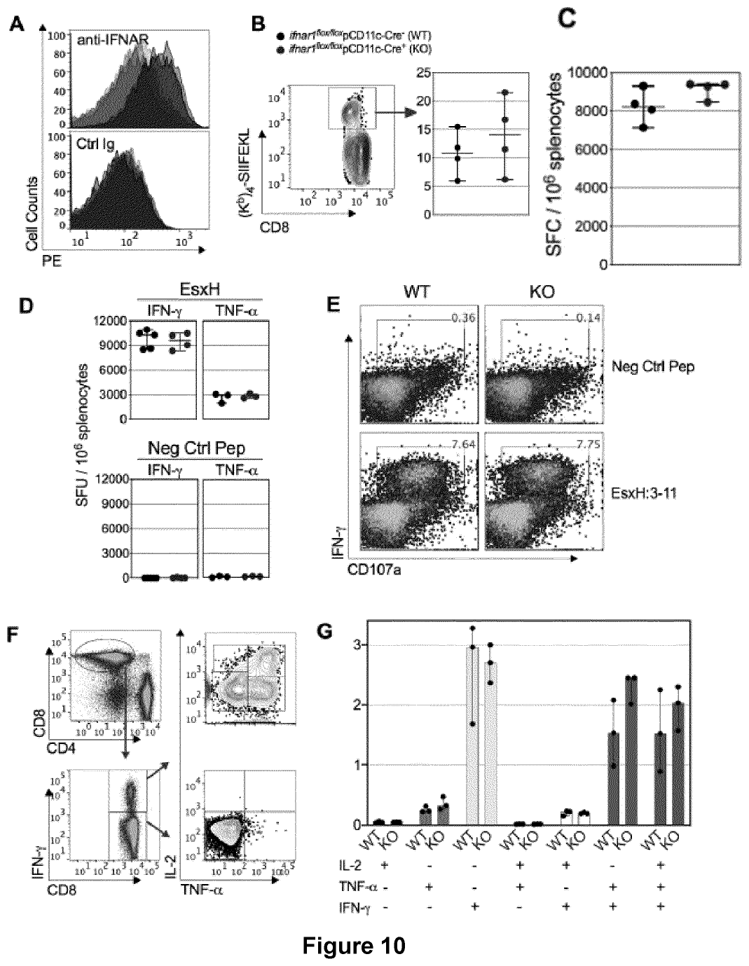

Based on the potency of LV at inducing IFN-I (30, 32) and the unique capacity of DC to activate naïve T cells, we then evaluated the dependence of LV-mediated CD8⁺ T-cell induction (12) on IFN-I signaling in DC. To this end, we used conditional C57BL/6 mutants, ifnar1^{flox/flox} pCD11c-Cre⁺ (with IFNAR-deficient DC) versus ifnar1^{flox/flox} pCD11c-Cre⁻ (with IFNAR-proficient DC), after preliminary confirmation that DC derived from the former displayed a large reduction of surface IFNAR expression (FIG. 10A). Mice, ifnar1^{flox/flox} pCD11c-Cre⁻ or Cre⁺, originated from the same litters, were immunized s.c. with 5×10⁷ Transduction Unit (TU) of LV::OVA or LV::li-EsxH. Eleven days post-immunization (dpi), tetramer staining, ELISPOT or Intracellular Cytokine Staining (ICS) assays detected in both mouse types strong and comparable CD8⁺ T splenocyte responses, specific to OVA (FIG. 10B-C) or EsxH (FIG. 10D-G), including similar proportions of IFN-γ⁺CD107a⁺ degranulating or polyfunctional CD8⁺ T cells. Therefore, the capacity of LV to induce CD8⁺ T-cell responses is not governed by IFNAR signaling in conventional DC.

The very slight DC stimulatory capacity of LV highlights the intrinsically minor inflammatory properties of these efficient vectors. In addition, the non-dependence of LV-mediated T-cell induction on DC signaling through the rare inflammatory mediators that they can induce (IFN-I) suggests that the underlying innate immune mechanisms are reduced to strict minimum.

### Substantial Capacity of the Optimized LV at Inducing Systemic and Mucosal CD4⁺ T-Cell Immunity

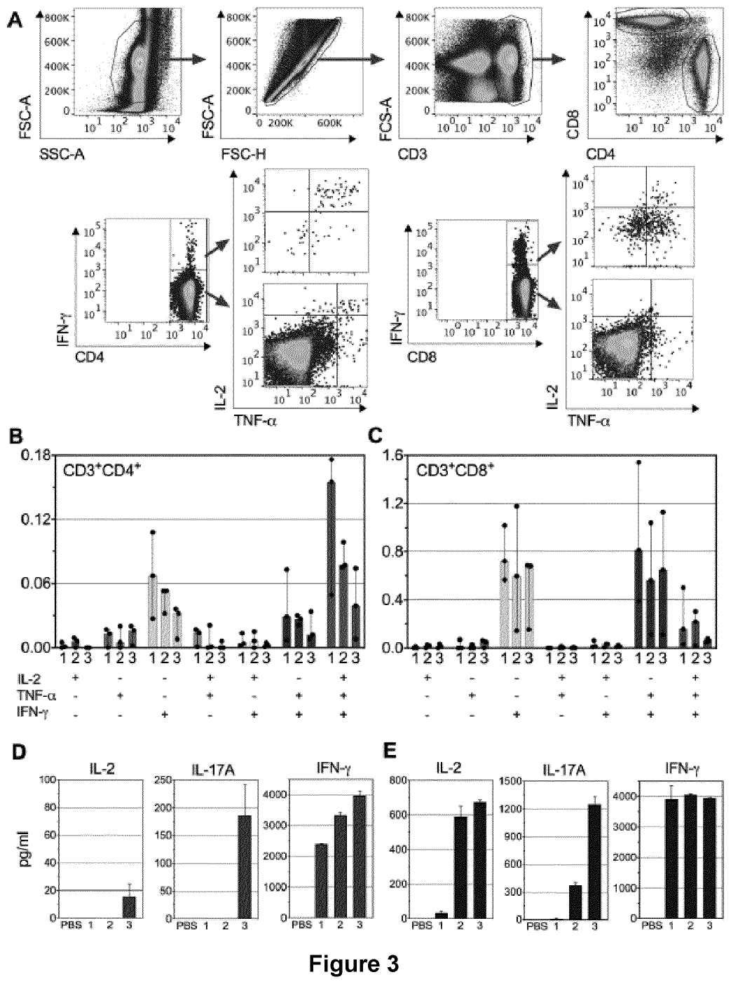

We then evaluated the potential of the optimized LV::li-EsxH at inducing CD4⁺ T-cell responses at the systemic or mucosal levels. Taking into the account the mild impact of LV on innate immune system and the so far uncharacterized CD4⁺ T-cell immunogenicity of this vector, we investigated the optimized LV::li-EsxH, either alone or adjuvanted with the pro-Th1 polyinosinic-polycytidylic acid (polyI:C) or the pro-Th1/Th17 cyclic Guanine-Adenine dinucleotide (cGAMP) (33). First, BALB/c mice were immunized s.c. with 5×10⁷ TU of LV::li-EsxH, alone or adjuvanted. At 11 dpi, ICS analysis detected remarkable amounts of EsxH-specific, Th1 cytokine-producing CD4⁺ (FIG. 3A, B), as well as CD8⁺ (FIG. 3A, C) T splenocytes. No significant impact of adjuvantation was observed in such responses induced by systemic immunization (FIG. 3B, C). Then, mucosal immunization of BALB/c mice was performed via intranasal (i.n.) route with 5×10⁷ TU of LV::li-EsxH, alone or adjuvanted. At 13 dpi, lung T cells were co-cultured with syngeneic DC loaded with EsxH: 74-88 or EsxH: 20-28 peptides, bearing respectively MHC-II or -I H-2^{d} T-cell epitopes (24, 25). Mucosal antigen-specific IL-2- or IL-17A-producing CD4⁺ T cells were only detected in the lungs of mice immunized with cGAMP-adjuvanted LV::li-EsxH (FIG. 3D). In parallel, mucosal antigen-specific IL-2- or IL-17A-producing CD8⁺ T cells were detected in the lungs of mice immunized with LV::li-EsxH combined with either adjuvant (FIG. 3E). Antigen-specific IFN-γ-producing lung CD4⁺ or CD8⁺ T cells were detected in all immunized groups (FIG. 3D, E).

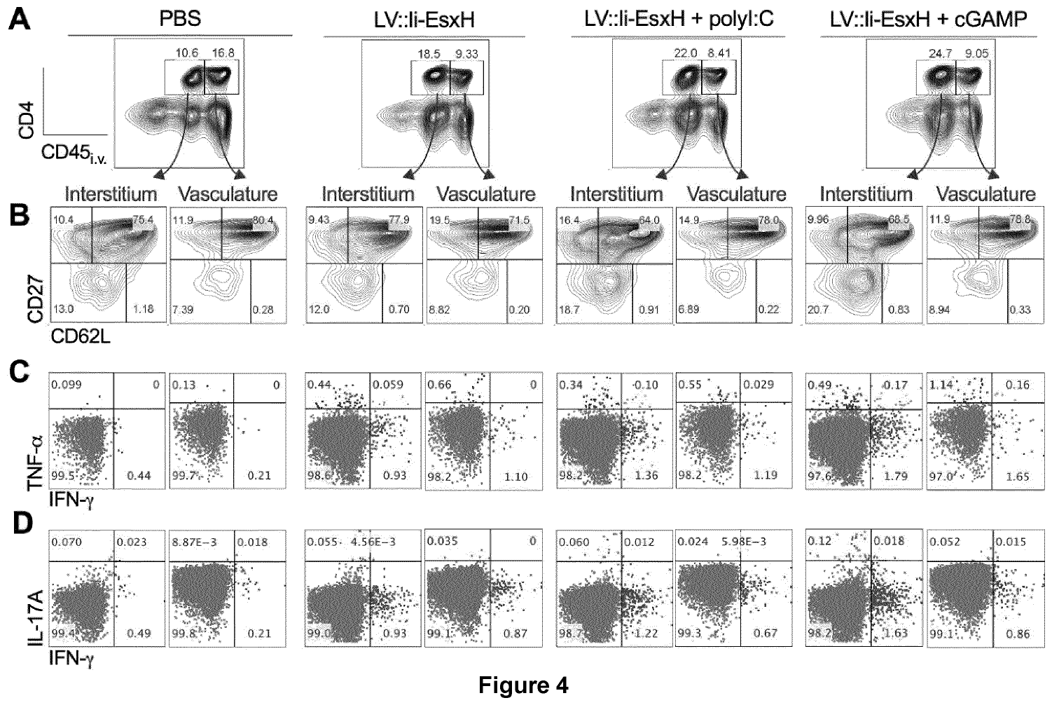

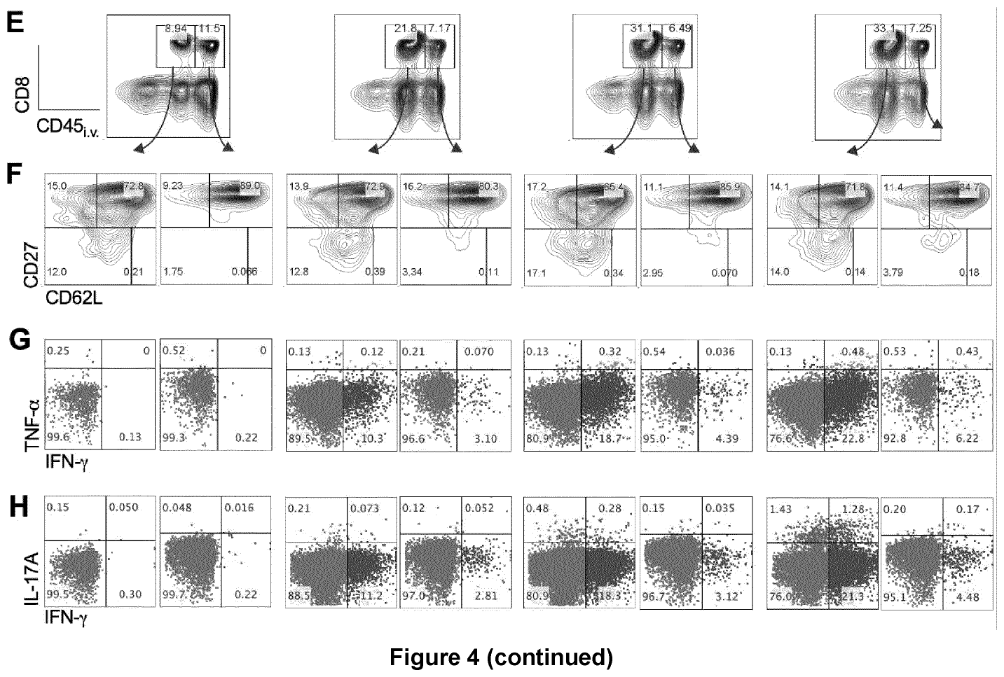

Intravenous (i.v.) injection of mice with PE-anti-CD45 mAb, 3 min before sacrifice, allows distinction of hematopoietic cells located inside the lung interstitium from those in the lung vasculature (34). Compared to the PBS-injected controls, mice immunized with LV::li-EsxH alone possessed notable percentages of CD45i.v CD4⁺ (FIG. 4A) or CD45_{i.v}⁻ CD8⁺ (FIG. 4E) T cells in the interstitium. This T-cell recruitment/expansion increased in mice immunized with adjuvanted LV::li-EsxH. Percentages of CD27⁻ CD62L⁻ recent migrant effectors, among interstitial CD4⁺ (FIG. 4B) or CD8⁺ (FIG. 4F) CD45_{i.v}⁻ T cells, were also higher in mice immunized with adjuvanted LV::li-EsxH. Notable amounts of antigen-specific IFN-γ/TNF-α-producing CD4⁺ (FIG. 4C) or CD8⁺ (FIG. 4G) T-cell effectors were detected in the interstitium of mice immunized with LV::li-EsxH alone and even more amounts of these T cells were detected in their counterparts immunized with adjuvanted LV::li-EsxH. Immunization with cGAMP-adjuvanted LV::li-EsxH generated Th17 (FIG. 4D) and Tc17 (FIG. 4H) cells, as detected in the interstitium and consistent with IL-17A released in the supernatants of lung T cells stimulated in vitro with EsxH: 74-88 or EsxH: 20-28 peptides (FIG. 3D, E). CD45i.v⁺ Th1 cytokine-producing CD4⁺ or CD8⁺ T cells were also detected in the vasculature (FIG. 4C, G), showing that the i.n. immunization also generated antigen-specific T cells that gain access to the blood circulation and can thus contribute to systemic immunity.

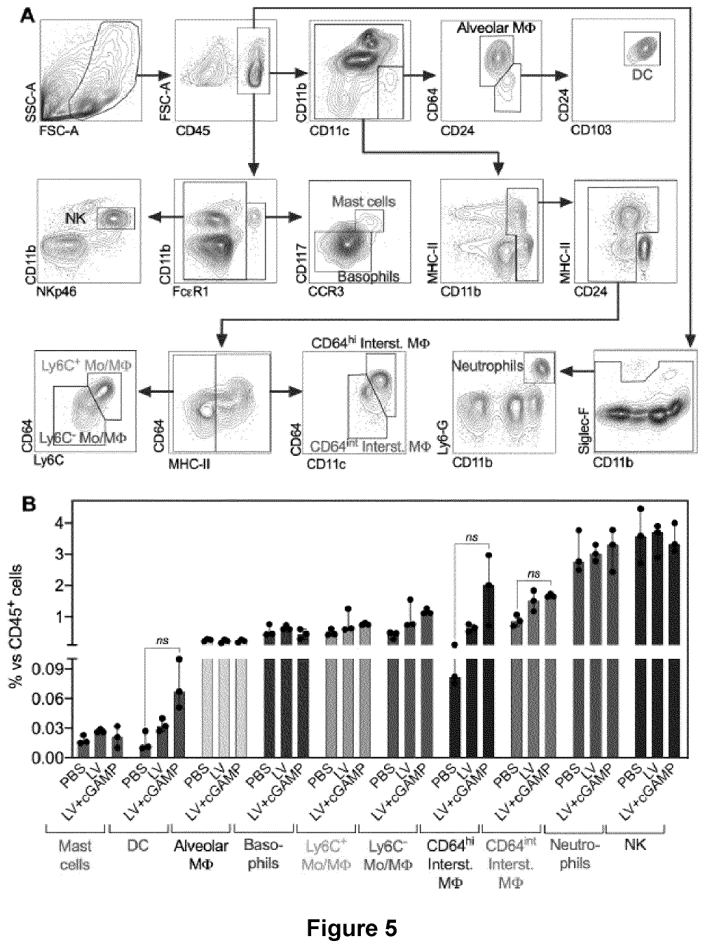

As determined by cytometry at 1 dpi, i.n. administration of LV alone did not have a significant impact on the proportions of various lung innate immune cell subsets, compared to PBS treatment alone (FIG. 5A, B). Following i.n. instillation of PolyI:C- or cGAMP-adjuvanted LV, minor and statistically unsignificant increases in the percentages of DC and interstitial macrophages were detected. Notably, the proportions of pro-allergenic mast cells or basophils, and inflammatory Ly6C⁺ macrophages/monocytes or neutrophils—potentially harmful in the context of mycobacterial infection (38)—remained unchanged in LV-treated mice.

### Generation of an Optimized Poly-Antigenic LV

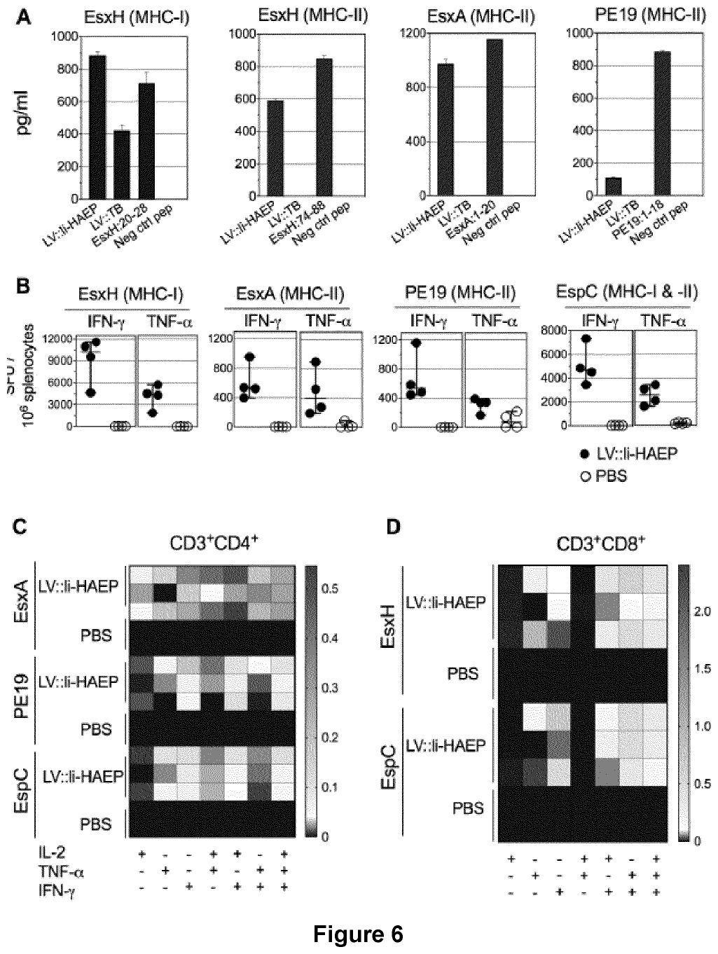

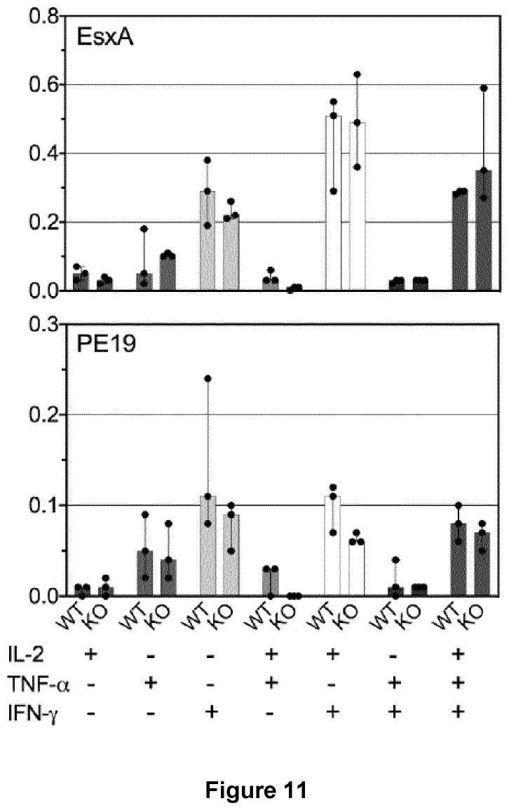

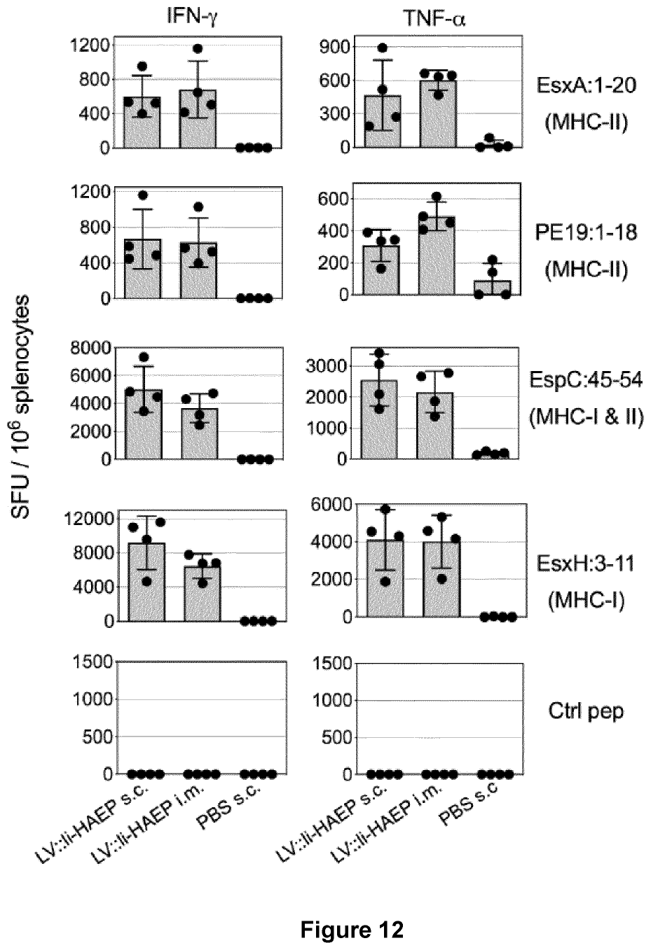

We then generated an optimized LV encoding for a fusion of li and juxtaposed sequences of EsxH, EsxA, EspC and PE19 (LV::li-HAEP) (Table S3, FIG. 5S). LV::li-HAEP-transduced DC were able to present the MHC-I- or -II-restricted epitopes of these immunogens to specific T-cell hybridomas (FIG. 6A). As determined by ELISPOT, in C57BL/6 mice, systemic s.c. immunization with 5×10⁸ TU of LV::li-HAEP alone induced IFN-γ/TNF-α-producing CD4⁺ or CD8⁺ T splenocytes specific to all included immunogens (FIG. 6B), with notable bi- or polyfunctionality in the both subsets (FIG. 6C, D). Like for LV-mediated CD8⁺ T-cell induction, with this optimized LV, we did not detect any dependence of CD4⁺ T-cell induction on DC IFNAR signaling (FIG. 11). We also established that immunization via s.c. or intramuscular (i.m.) systemic route with LV::li-HAEP alone resulted in comparable IFN-γ or TNF-α CD4⁺ and CD8⁺ T splenocyte responses (FIG. 12).

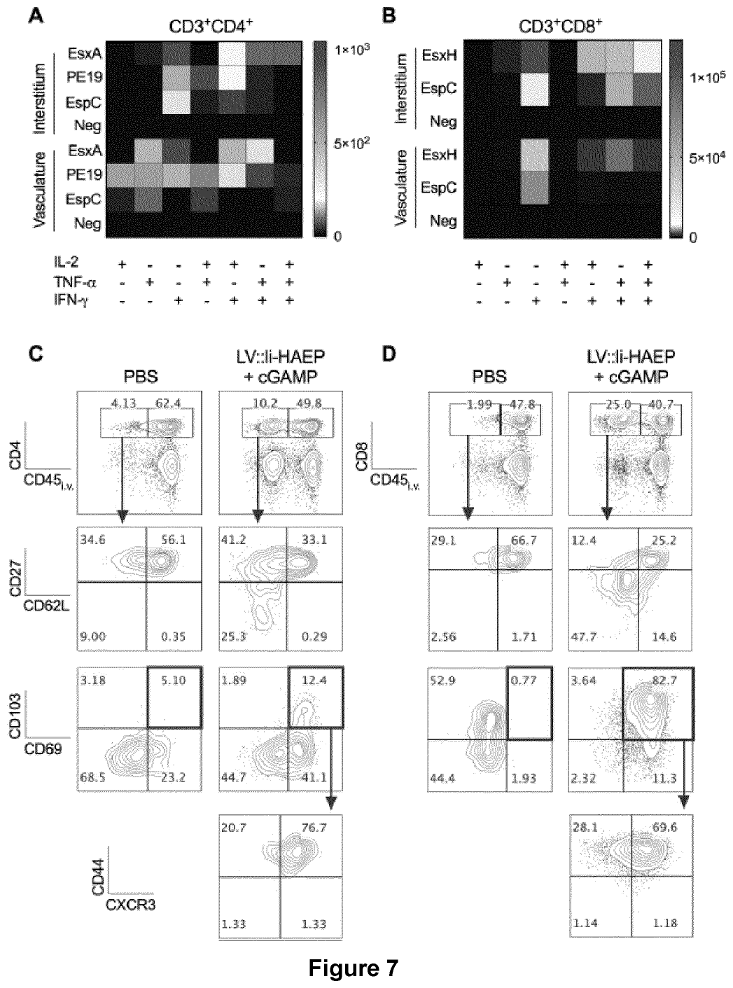

Mucosal i.n. immunization of C57BL/6 mice with cGAMP-adjuvanted LV::li-HAEP elicited (poly) functional CD4⁺ (FIG. 7A) or CD8⁺ (FIG. 7B) T cells specific to each of the 4 Mtb antigens in lung interstitium, and also at a lesser extent in the vasculature. The CD45_{i.v.}⁻ interstitial CD4⁺ or CD8⁺ T subset in the vaccinated mice contained increased proportions of CD27⁻ CD62L⁻ migrant effectors and CD69⁺ CD103⁺ lung-tissue resident cells (FIG. 7C). The majority of CD69⁺ CD103⁺ CD4⁺ T cells displayed a CD44⁺ CXCR3⁺ phenotype (FIG. 7C bottom), reminiscent of CD8⁺ T-cell resident-memory phenotype (39, 40).

### Evaluation of the Protective Booster Potential of the Optimized Poly-Antigenic LV

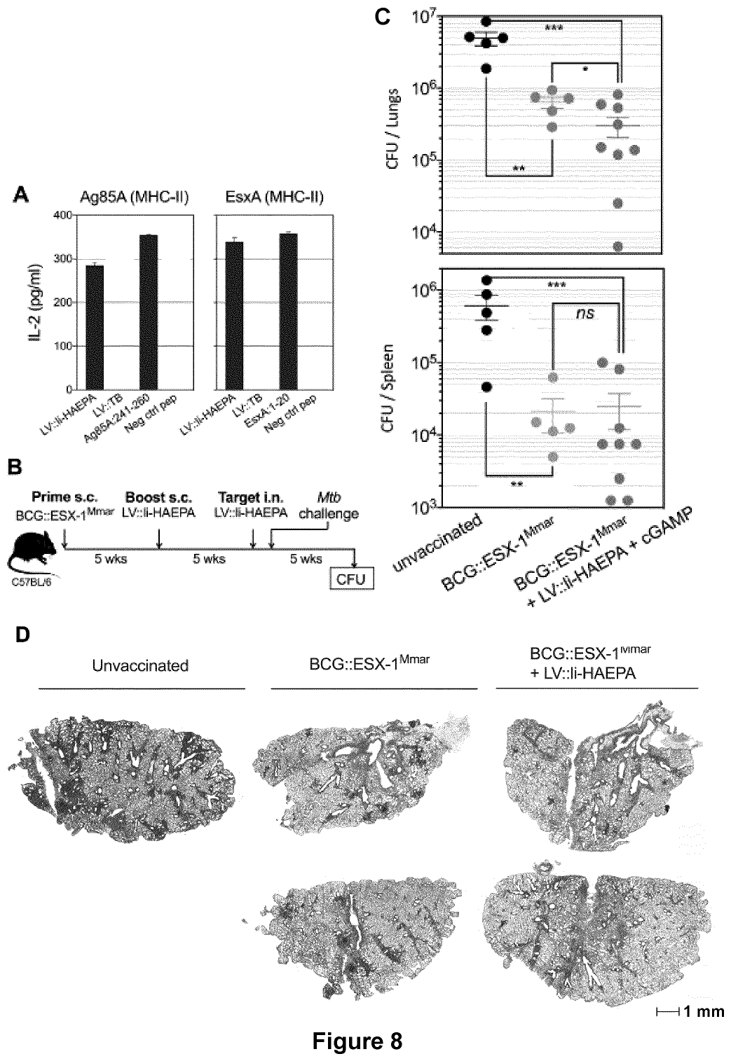

Considering the interest of Ag85A/B antigens as vaccine targets (FIG. 1), to maximize the boosting potential of the elaborated vector, we added the Ag85A: 241-260 immunogenic region (41, 42) to the C-ter end of HAEP in LV::li (LV::li-HAEPA) (Table S3, FIG. 13). LV::li-HAEPA-transduced DC were able to induce MHC-II-restricted presentation of Ag85A: 241-260 to specific T-cell hybridoma (FIG. 8A), in addition to the presentation of the other Mtb antigens, as exemplified by EsxA and as detected by specific T-cell hybridoma (ref 23) To evaluate the booster efficacy of LV::li-HAEPA, C57BL/6 mice were either left unvaccinated or primed s.c. at week 0 with 1×10⁶ CFU of BCG::ESX-1^{Mmar} vaccine candidate with increased protective capacity compared to the parental BCG (8) (FIG. 8B). The advantage of this live-attenuated vaccine candidate over BCG in prime immunization is linked to its capacity to secrete EsxA and EspC via the orthologous ESX-1 T7SS, which allows here to boost the T-cell responses against all the Mtb antigens included in the optimized poly-antigenic LV. A group of BCG::ESX-1^{Mmar} primed mice was boosted s.c. with 5×10⁸ TU of cGAMP-adjuvanted LV::li-HAEPA at week 5, and then again boosted i.n. at week 10 with the same amount of cGAMP-adjuvanted LV::li-HAEPA in order to attract the induced immune effectors to the lung mucosa. At week 12, mice were challenged with ≈200 CFU of virulent Mtb H37Rv strain via aerosol and mycobacterial burdens were determined in the lungs and spleen at week 17. The lung mycobacterial load average in the primed-boosted mice was decreased by ≈2.5 log 10 compared to unvaccinated controls (Mann-Whitney test, p value=0,0005), and by ≈1 log₁₀ compared to their BCG::ESX-1^{Mmar}-vaccinated counterparts (Mann-Whitney test, p value=0,0415) (FIG. 8C). This significant decrease in the primed and boosted mice compared to the mice vaccinated only with BCG::ESX-1^{Mmar} seems nevertheless without major impact on lung histopathology (FIG. 8D). In the spleen, the boost with cGAMP-adjuvanted LV::li-HAEPA led to a net trend to reduced mycobacterial loads, which however did not reach statistical significance. This could be explained by the particularly strong and hardly improvable protective effect of ESX-1-complemented BCG strains against dissemination to the spleen in the mouse and guinea pig models (22, 43, 44).

### Discussion

Extensive investigations of human immune cells by “omics” approaches in healthy donors or individuals with latent versus active TB allowed identification of biomarkers for developing host response-based diagnosis of active TB (45). However, these studies did not provide a thorough view of multifactorial processes which result in immune failure of granulomas and progression to active TB. Therefore, reliable correlates of optimal protection, and causative biomarkers of non-progression of latent to active TB remain largely elusive. In this context, rational design of new generation of TB vaccines is challenging (46). One consensus in the domain consists of prime-boost immunization approaches. BCG displayed an excellent safety record over the last 80 years and shows a high rate of protector effect against disseminated forms of TB in children. Therefore, the use of: (i) BCG or an improved live-attenuated vaccine for priming, and (ii) subunit vaccine candidates for boosting, represent a promising strategy.

Viral vectors, notably Modified Vaccinia Ankara (MVA) or adenoviral vectors have been used in immunization against Mtb (7). Despite its remarkable success in pre-clinical animal models, an MVA encoding Ag85A, was poorly immunogenic in clinical trials and was unable to induce protection (47). Another LV encoding Ag85A, together with an NF-kB activator, induced systemic and mucosal T-cell immunity, but did not afford protection against a BCG challenge in the mouse model (48). In BCG-primed mice, a boost with an LV encoding an Ag85B-PPE57 fusion, increased the amplitude of T-cell responses and protection against a high-dose i.v. Mtb challenge (49). In these studies, the LV encoded only for one or two Mtb antigens and were not optimized to target the antigens to the MHC-II presentation pathway, which can explain their poor capacity to induce protection against Mtb. In fact, despite their remarkable ability to target endogenously produced antigens into the MHC-I pathway of the transduced antigen presenting cells, viral vectors, including LV, mostly fail to deliver antigens to the MHC-II machinery for CD4⁺ T-cell induction. Here, we generated a new generation of LV in which the genes encoding multiple potent Mtb antigens were engineered to allow trafficking of the resulted fusion proteins through the MHC-II pathway. Addition of the li or TfR at the N-ter of a single or a poly-antigenic protein, achieved proper antigen routing to the MHC-II machinery and robust triggering of CD4⁺ T cells, without any propensity to reduce MHC-I presentation or CD8⁺ T-cell induction. However, li fusion to the N-ter of protein sequences might not be always sufficient and preservation of the native tertiary structure of the resulting proteins seems to matter as well. For instance, LV encoding for a fusion of li and a cluster of predicted T-cell epitopes, derived from EsxH, EsxA, EspC and PE19, failing to preserve protein folding and enriching the sequences in hydrophobic residues, did not induce efficient antigen routing to MHC-II machinery.

The choice of the Mtb immunogens included in the poly-antigen inserted in the optimized LV was based on their direct relationship with the mycobacterial virulence in vivo and active secretion by the ESX-1, -3, -5 T7SS or Tat systems, throughout various TB phases (16, 17). Among these proteins, PE19 is of particular interest. As a single antigen, PE19 harbors T-cell epitopes which are shared with its several homologs. The Mtb genome contains up to one hundred of pe (and ppe) genes. The resulted PE/PPE proteins, named after their N-ter PE or PPE motifs (18, 50, 51), form large multigenic families of proteins, which are secreted or cell wall-attached and many are related to pathogenic potential (18-21). Resulting from ancestral gene duplication, PE/PPE proteins display substantial sequence homologies and thus share plethora of T-cell epitopes (42). The arbitrary insertion of the pe/ppe genes all over the Mtb genome led to their expression by an array of independent promoters, which generates unprecedented degrees of variability in their expression profiles at distinct infection phases (52). This situation can readily generate consecutive display of groups of shared PE (/PPE) epitopes, during various TB phases (42, 53-55).

As we recently demonstrated with LV-based vaccination against SARS-COV-2 (56), systemic immune responses, even of high quality, may not always reach the site of the infection in the lung mucosa to prevent replication of pulmonary pathogens. Mucosal immunity, including antibodies and tissue-resident lymphocytes, have been shown to be instrumental in pathogen clearance from the respiratory tracts (56-60). In TB vaccination, our previous results demonstrated the advantages of i.n. immunization with Esx or PE/PPE antigens in various formulations (9, 61). Moreover, the protection against pulmonary TB has been correlated with the presence of antigen-specific resident-memory CD4⁺ T cells (62-65). Here, we thoroughly characterized the functions and phenotype of CD4⁺ and CD8⁺ T cells, induced through systemic or i.n. administration of the optimized LV encoding EsxH or HAEP(A) poly-antigen. Most notably, mucosal immunization induced lung CD4⁺ and CD8⁺ T cells with polyfunctional effector features, accompanied by activated, tissue-resident and memory phenotypes. When formulated with cGAMP adjuvant and administered via i.n., the optimized LV also triggered lung Th17 and Tc17 responses with prospective implications in the protection against Mtb (66, 67).

The very mild impact of LV on DC maturation in vitro, and the very slight modification of the lung innate immune cell composition subsequent to i.n. administration of LV alone, indicate the intrinsically low inflammatory properties of these vectors. Interestingly, DC signaling through IFN-I, i.e., the rare inflammatory factors induced by LV, is not involved in CD4⁺ or CD8⁺ T-cell induction by these vectors. This suggests a minimalist involvement of innate immune pathways engaged by LV to induce robust T-cell immunity. These characteristics, together with the non-replicative property of LV, reflect favorably on their safety for veterinary or human vaccination, notably via the mucosal pathways. In addition, due to the mucosal barrier, i.n. immunization could even generate minimized systemic adverse effects (68).

Finally, we investigated the protective potential of a vaccination approach based on priming with the improved live-attenuated vaccine, BCG::ESX-1^{Mmar} (8), and boosting with the optimized LV::li-HAEPA formulated in cGAMP in the prophylactic C57BL/6 mouse TB model. BCG::ESX-1^{Mmar} per se triggered a substantial reduction in the Mtb loads in the lungs and spleen, while LV boosting via systemic and nasal routes achieved significant additional decrease of bacterial loads in the lungs, accompanied by a net trend to weakened dissemination to the spleen. These data provide the proof-of-concept evidences that, in the context of LV, not only single small antigens like EsxH, but also fusion of multiple antigens, fused to li are able to gain access to the MHC-II presentation pathway to induce CD4⁺ T cells, without reduction of CD8⁺ T-cell triggering. In addition, i.n. immunization with the optimized LV induces recruitment and establishment of poly-specific lung CD4⁺ and CD8⁺ T-cell immunity with resident-memory phenotype. This approach can optionally be improved by the addition of appropriate adjuvants.

Whether the i.n. immunization involves the mediastinal lymph nodes, immune cells directly recruited and located into the lung parenchyma or the highly organized ectopic lymphoid-like structures of “tertiary lymphoid organs” remains to be uncovered. The latter mimic the immune germinal centers in the mucosal tissues, providing local and controlled inflammation and an optimal environment for innate and adaptive immune cell cross-talk to reinforce anti-microbial host immunity at the site of the potential 5 infection (74).

The non-replicative and very weakly inflammatory properties of LV, now optimized to induce CD4⁺ T-cell responses, predict these vectors as tools of choice for mucosal vaccination, especially via the i.n. route. The prospects for development of these LV-based strategies go far beyond mycobacterial infections, extending the approach to acute or chronic respiratory infectious diseases.

### Materials and Methods

### Construction of Transfer Plasmids Encoding Mtb (Poly) Antigen and LV Generation

Codon-optimized genes encoding EsxH alone or in fusion with the II, TfR, and MITD or encoding II-HAEP or II-HAEPA were synthetized by Eurofins were then cloned downstream of the “SP1” promoter: (i) based on human 32 microglobulin (β2m) promoter which derives antigen expression predominantly in immune cells and notably activated APCs (70), and (ii) containing inserted/substituted regions originated from the CMV promoter albeit with minimal proximal enhancers and thus improved vector safety (our unpublished results). The promoter is located between BamHI and Xhol sites of the pFLAPAU3 transfer plasmid (14) (FIG. 13) containing a mutated WPRE (Woodchuck Posttranscriptional Regulatory Element) sequence to increase gene transcription. Production and titration of LV were performed as described elsewhere (56)

### Mycobacteria

Mtb (H37Rv strain) or BCG: ESX-1^{Mmar} (8), were cultured to exponential phase in Dubos broth, complemented with Albumine, Dextrose and Catalase (ADC, Difco, Becton Dickinson, Le Pont-de-claix, France). Non-Beijing and Beijing clinical Mtb isolates, representative of the most prevalent genotypes in France, have been submitted to the National Reference Centre for TB for drug-resistance characterization and Mycobacterial Interspersed Repetitive-Unit-Variable-Number Tandem-Repeat (MIRU-VNTR) genotyping (75). Mtb clinical isolates were grown in Dubos broth, complemented with oleic ADC (OADC, Difco). Titers of the mycobacterial cultures were determined by OD₆₀₀ measuring. Experiments with pathogenic mycobacteria were performed in BSL3, following the hygiene and security recommendations of Institut Pasteur.

### Detection of MHC-I or -II Restricted Antigenic Presentation In Vitro

Histocompatible bone-marrow derived DC were plated at 5×10⁵ cells/well in 24-well plates in RPMI 1640 containing 5% FBS. When adherent, cells were transduced with LV vectors, or were loaded with 1 μg/ml of homologous or control synthetic peptides. At 24 h post infection 5×10⁵ appropriate T-cell hybridomas were added and the co-culture supernatants were assessed for IL-2 production at 24h by ELISA. In this assay, the amounts of released IL-2 is proportional to the efficacy of antigenic presentation by MHC molecules. The peptides harboring MHC-I or -II-restricted epitopes were synthesized by Proteogenix (Schiltigheim, France) and were reconstituted in H₂O containing 5% Di-Methyl Sulfoxyd (DMSO) (Sigma-Aldrich). When indicated antigenic presentation was assessed by use of reporter T-cell hybridomas, transduced to emit fluorescent signals subsequent to TCR triggering, as recently described (23).

### Mice, Immunization

Female BALB/c (H-2^{d}) and C57BL/6 (H-2^{b}) (Janvier Labs, Le Genest-Saint-Isle, France) were immunized after at least one week of acclimatation, with the indicated dose of LV contained in 50 μl/mouse for i.m. injection, in 200 μl/mouse for s.c. at the basis of the tail, or in 20 μl/mouse for i.n. instillation. The i.n. administration was performed under general anesthesia, obtained by i.p. injection of 100 μl of PBS containing weight-adapted quantities of Imalgène1000 (Kétamine, i.e., 100 mg/kg, Merial, France) and Rompun 2% (Xylazine solution, 10 mg/kg, Bayer, Germany). When indicated LV was adjuvanted with 10 ug/mouse of polyI:C or cGAMP (Invivogen).

The hemizygous C57BL/6 (H-2^{b}) mice, carrying the gene encoding Cre DNA recombinase, under the regulation of murine CD11c promoter (76), were crossed with C57BL/6 mice homozygous for the “floxed” ifnar1 allele (77) to obtain litters of homozygous ifnar1^{flox/flox} mice that carry or not the Cre transgene. In ifnar1^{flox/flox} pCD11c-Cre⁺ mice, with the exception of CD11c-expressing plasmacytoid DC, all other DC populations lacked IFNAR1 (77). The breeding was performed at the central animal facilities of Institut Pasteur, under SPF conditions.

All the mice were used between the age of 8 and 16 weeks, in accordance with the European and French directives (Directive 86/609/CEE and Decree 87-848 of 19 Oct. 1987), after approval by the Institut Pasteur Safety, Animal Care and Use Committee, under local ethical committee protocol agreement #CETEA 2013-0036 and CETEA 2012-0005 (APAFIS #14638-2018041214002048).

### Intracellular Cytokine Staining

Splenocytes from immunized mice were obtained by tissue homogenization and passage through 100-μm nylon filters (Cell Strainer, BD Biosciences) and were plated at 4×10⁶ cells/well in 24-well plates. Lungs were treated with 400 U/ml type IV collagenase and DNase I (Roche) for 30 min at 37° C. and homogenized by use of GentleMacs (Miltenyi). Cells were then filtered through 70-μm nylon filters (Cell Strainer, BD Biosciences), and centrifuged for 20 min at 3000 rpm at RT without brake on Ficoll gradient medium (Lympholyte M, Cedarlane Laboratories). Lung T-cell-enriched fractions were co-cultured at 4×10⁶ cells/well with histocompatible bone-marrow-derived DC (8×10⁵ cells/well) in 24-well plates. Splenocytes or lung T cells were co-cultured during 6h in the presence of 10 μg/ml of homologous or control peptide, 1 μg/ml of anti-CD28 (clone 37.51) and 1 μg/ml of anti-CD49d (clone 9C10-MFR4.B) mAbs (BD Biosciences). During the last 3h of incubation, cells were treated with a mixture of Golgi Plug and Golgi Stop, both from BD Biosciences. When indicated, PE-Cy7-anti-CD107a (clone 1D4B, BioLegend) mAb was also added to the cultures at this step. Cells were then collected, washed with PBS containing 3% FBS and 0.1% NaN₃ (FACS buffer) and incubated for 25 min at 4° C. with a mixture of FcγII/III receptor blocking anti-CD16/CD32 (clone 2.4G2) and APC-eFluor780-anti-CD3E (clone17A2), eF450-anti-CD4 (clone RM4-5), BV711-anti-CD8 (clone 53-6.7) mAbs (BD Biosciences or eBioscience). Cells were washed twice in FACS buffer, then permeabilized by use of Cytofix/Cytoperm kit (BD Bioscience). Cells were then washed twice with PermWash 1× buffer from the Cytofix/Cytoperm kit and incubated with a mixture of AF488-anti-IL-2 (clone JES6-5H4, BD Biosciences), PE/Dazzle 594-anti-TNF-α (MP6-XT22, BioLegend), and APC-anti-IFN-γ (clone XMG1.2, BD Biosciences) mAbs or a mixture of appropriate control Ig isotypes, during 30 min at 4° C. Cells were then washed twice in PermWash and once in FACS buffer, then fixed with Cytofix (BD Biosciences) overnight at 4° C. Cells were acquired in an Attune NxT cytometer system (Invitrogen) and data analysis was performed using FlowJo software (Treestar, OR, USA).

### Lung Cell Phenotyping

Lymphocyte-enriched lung cells from mice, injected i.v. with PE-anti-CD45 (clone 30-F11, BioLegend) 3 min before sacrifice, were prepared as described above and stained with a mixture of APC-eFluor780-anti-CD3 (clone17A2, eBioscience), eF450-anti-CD4 (clone RM4-5, eBioscience), BV711-anti-CD8 (clone 53-6.7, BD Biosciences) mAbs, with either: (i) PE-Cy7-anti-CD27 (clone LG.7F9, eBioscience) and AF700-anti-CD62L (clone MEL-14, BD Biosciences) mAbs, or (ii) BV605-anti-CD69 (clone H1.2F3, BioLegend), FITC-anti-CD103 (clone 2E7, BioLegend), PE-Cy7-anti-CD49a (clone HM1, BioLegend), AF700-anti-CD44 (clone IM7, BioLegend) and APC-Fire750-anti-CXCR3 (clone CXCR3-173, BioLegend) mAbs, all in the presence of FcγII/III receptor blocking anti-CD16/CD32 (BD Biosciences). After 25 min incubation at 4° C., the cells were washed twice in FACS buffer and fixed by incubation with Cytofix (BD Bioscience) overnight at 4° C. Cytometric analysis of lung innate immune cells was recently detailed elsewhere (56).

### Elispot Assay

Splenocytes from individual mice were homogenized and filtered through 100 μm-pore filters and centrifuged at 1500 rpm during 5 min. Cells were then treated with Red Blood Cell Lysing Buffer (Sigma), washed twice in PBS and counted in a MACSQuant-10 cytometer (Miltenyi Biotec). Splenocytes were then plated at 1×10⁵ cells/well in 200 μl of RPMI-GlutaMAX, containing 10% heat-inactivated FBS, 100 U/ml penicillin and 100 μg/ml streptomycin, 1×10⁻⁴ M non-essential amino-acids, 1% vol/vol HEPES, 1×10⁻³ M sodium pyruvate and 5×10⁻⁵ M of β-mercaptoethanol in ELISPOT plates (Mouse IFN-γ or TNF-α ELISPOTPLUS, Mabtech). Cells were left unstimulated or were stimulated with 2 μg/ml of appropriate synthetic peptides (Proteogenix) or 2.5 μg/ml of

Concanavalin A (Sigma), as a functionality control. For each mouse, the assay was performed in triplicates, according to the manufacturer's recommendations. Plates were analyzed in an ELR04 ELISPOT reader (AID, Strassberg, Germany).

### Protection Assay

C57BL/6 mice were primed s.c. with 1×10⁶ CFU/mouse of BCG: ESX-1^{Mmar} (8) at day 0, boosted s.c. with 5×10⁸ TU/mouse of adjuvanted LV at week 5, and boosted again i.n. with 5×10⁸ TU/mouse of adjuvanted LV at week 10. The immunized mice, as well as age-matched, unvaccinated controls, were challenged 2 weeks after the i.n. boost by use of a homemade nebulizer via aerosol, as previously described (9). Briefly, 5 ml of a suspension of 1.7×10⁶ CFU/ml of H37Rv Mtb strain were aerosolized to deliver an inhaled dose of ≈200 CFU/mouse. The infected mice were placed in isolator in BSL3 facilities at Institut Pasteur. Five weeks later, lungs or spleen of the infected mice were homogenized by using a MillMixer homogenizer (Qiagen, Courtaboeuf, France) and serial 5-fold dilutions prepared in PBS were plated on 7H11 Agar complemented with ADC (Difco, Becton Dickinson). CFU were counted after 3 weeks of incubation at 37° C. Significance of inter-group CFU differences was determined by Mann-Whitney t-test by use of Prism v8.01 (GraphPad Software, Inc.).

TABLE S1
Non-Beijing or Beijing Mtb clinical isolates from MIRU-VNTR,
tested for the intra-phagocyte Ag85A/B and EsxA expression.

| Corresponding Number | Clinical Isolate |
|---|---|
| on Heat Maps | No. lineage |
| | Non Beijing |
| 1 | 661603069400 |
| 2 | 661610071089 |
| 3 | 661702081724 |
| 4 | 661612077758 |
| 5 | 661708094529 |
| 6 | 661801095406 |
| 7 | 661805075095 |
| 8 | 661412076132 |
| 9 | 661404013957 |
| 10 | 661407086878 |
| 11 | 661405051259 |
| 12 | 661404032252 |
| 13 | 661408059503 |
| 14 | 661409034117 |
| 15 | 661410060829 |
| | Beijing |
| 1 | 661701115366 |
| 2 | 661711076067 |
| 3 | 661501008044 |
| 4 | 661508077689 |
| 5 | 661611080910 |
| 6 | 661602107040 |
| 7 | 661604058591 |
| 8 | 661609072250 |
| 9 | 661612100794 |
| 10 | 661701037389 |
| 11 | 661706099221 |
| 12 | 661707013366 |
| 13 | 661707023069 |
| 14 | 661709001208 |
| 15 | 661709065148 |
| 16 | 661805075112 |
| 17 | 661711117831 |
| 18 | 661406088111 |
| 19 | 661406088102 |
| 20 | CR 1201 6122 |
| 21 | CR 1104 7352 |
| 22 | CR 1005 5423 |
| 23 | CR 1010 5929 |
| 24 | CR 1008 5736 |
| 25 | CR 1010 5930 |
| 26 | 661305027157 |
| 27 | CR 0910 6843 |
| 28 | CR 0910 6844 |
| 29 | CR 0905 6418 |
| 30 | CR 0905 6417 |
| 31 | CR 1211 7109 |

TABLE S2
MHC-I or -II restricted T-cell hybridomas specific to the
selected Mtb immunogens

| | MHC | | T-cell | Restricting |
|---|---|---|---|---|
| Antigen | epitope | Epitope sequence | hybridoma | element |
| EsxH | 20-28 | GYAGTLQSL | YB8 | K^{d} |
| | | (SEQ ID No. 15) | | |
| EsxH | 74-88 | STHEANTMAMMARDT | 1H2 | I-A^{d} |
| | | (SEQ ID No. 16) | | |
| EsxA | 1-20 | MTEQQWNFAGIEAAASAIQG | NB11 | I-A^{b} |
| | | (SEQ ID No. 17) | | |
| PE19 | 1-18 | MSFVTTQPEALAAAAANL | IF6 | I-A^{b} |
| | | (SEQ ID No. 18) | | |
| Ag85A | 241-260 | QDAYNAGGGHNGVFDFPDSG | DE10 | I-A^{b} |
| | | (SEQ ID No. 19) | | |

TABLE S3
Sequences of fused li and selected Mtb antigens, inserted in LV

| | Insert | |
|---|---|---|
| Poly-antigenic LV | length (a.a.) | Sequence |
| LV::li-EsxH-EsxA-EspC- | 617 | MDDQRDLISNHEQLPILGNRPREPERCSRGALYTGVSVLVA |
| PE19 | | LLLAGQATTAYFLYQQQGRLDKLTITSQNLQLESLRMKLPK |
| (LV::li-HAEP) | | SAKPVSQMRMATPLLMRPMSMDNMLLGPVKNVTKYGNM |
| (SEQ ID No. 1) | | TQDHVMHLLTRSGPLEYPQLKGTFPENLKHLKNSMDGVN |
| | | WKIFESWMKQWLLFEMSKNSLEEKKPTEAPPKEPLDME |
| | | DLSSGLGVTRQELGQVTLGGGDSQIMYNYPAMLGHAGDMA |
| | | GYAGTLQSLGAEIAVEQAALQSAWQGDTGITYQAWQAQWN |
| | | QAMEDLVRAYHAMSSTHEANTMAMMARDTAEAAKWGGN |
| | | TEQQWNFAGIEAAASAIQGNVTSIHSLLDEGKQSLTKLA |
| | | AAWGGSGSEAYQGVQQKWDATATELNNALQNLARTIS |
| | | EAGQAMASTEGNVTGMFANNGGTENLTVQPERLGVLAS |
| | | HHDNAAVDASSGVEAAAGLGESVAITHGPYCSQFNDTLNV |
| | | YLTAHNALGSSLHTAGVDLAKSLRIAAKIYSEADEAWRKAI |
| | | DGLFTNNDDSFVTTQPEALAAAAANLQGIGTTMNAQNAAA |
| | | AAPTTGVVPAAADEVSALTAAQFAAHAQMYQTVSAQAAAIH |
| | | EMFVNTLVASSGSYAATEAANAAAAG |
| LV::li-EsxH-EsxA-EspC- | 636 | MDDQRDLISNHEQLPILGNRPREPERCSRGALYTGVSVLVA |
| PE19-Ag85A:241-260 | | LLLAGQATTAYFLYQQQGRLDKLTITSQNLQLESLRMKLPK |
| (LV::li-HAEPA) | | SAKPVSQMRMATPLLMRPMSMDNMLLGPVKNVTKYGNM |
| (SEQ ID No. 3) | | TQDHVMHLLTRSGPLEYPQLKGTFPENLKHLKNSMDGVN |
| | | WKIFESWMKQWLLFEMSKNSLEEKKPTEAPPKEPLDME |
| | | DLSSGLGVTRQELGQVTLGGGDSQIMYNYPAMLGHAGDMA |
| | | GYAGTLQSLGAEIAVEQAALQSAWQGDTGITYQAWQAQWN |
| | | QAMEDLVRAYHAMSSTHEANTMAMMARDTAEAAKWGGN |
| | | TEQQWNFAGIEAAASAIQGNVTSIHSLLDEGKQSLTKLA |
| | | AAWGGSGSEAYQGVQQKWDATATELNNALQNLARTIS |
| | | EAGQAMASTEGNVTGMFANNGGTENLTVQPERLGVLAS |
| | | HHDNAAVDASSGVEAAAGLGESVAITHGPYCSQFNDTLNV |
| | | YLTAHNALGSSLHTAGVDLAKSLRIAAKIYSEADEAWRKAI |
| | | DGLFTNNDDSFVTTQPEALAAAAANLQGIGTTMNAQNAAA |
| | | AAPTTGVVPAAADEVSALTAAQFAAHAQMYQTVSAQAAAIH |
| | | EMFVNTLVASSGSYAATEAANAAAAQDAYNAGGGHNGVFD |
| | | FPDSG |
| LV::li-EsxH | 315 | MDDQRDLISNHEQLPILGNRPREPERCSRGALYTGVSVLVA |
| (SEQ ID No. 5) | | LLLAGQATTAYFLYQQQGRLDKLTITSQNLQLESLRMKLPK |
| | | SAKPVSQMRMATPLLMRPMSMDNMLLGPVKNVTKYGNM |
| | | TQDHVMHLLTRSGPLEYPQLKGTFPENLKHLKNSMDGVN |
| | | WKIFESWMKQWLLFEMSKNSLEEKKPTEAPPKEPLDME |
| | | DLSSGLGVTRQELGQVTLGAGAMSQIMYNYPAMLGHAGDM |
| | | AGYAGTLQSLGAEIAVEQAALQSAWQGDTGITYQAWQAQW |
| | | NQAMEDLVRAYHAMSSTHEANTMAMMARDTAEAAKWGG |
| TfR₁₋₁₁₈-EsxH | 218 | MMDQARSAFSNLFGGEPLSYTRFSLARQVDGDNSHVEMK |
| (SEQ ID No. 7) | | LAVDEEENADNNTKANVTKPKRCSGSICYGTIAVIVFFLIGF |
| | | MIGYLGYCKGVEPKTECERLAGTESPVREEPGEDFPAGAGA |
| | | MSQIMYNYPAMLGHAGDMAGYAGTLQSLGAEIAVEQAALQS |
| | | AWQGDTGITYQAWQAQWNQAMEDLVRAYHAMSSTHEANT |
| | | MAMMARDTAEAAKWGG |

Double-underlined sequences are junctions inserted to avoid neo-epitope generation.

### REFERENCES

1. WHO. 2018. http://www.who.int/tb/publications/global_report/en/.
2. Dockrell H M, Smith S G. 2017. What Have We Learnt about BCG Vaccination in the Last 20 Years? Front Immunol 8:1134.
3. Ernst J D. 2018. Mechanisms of M. tuberculosis Immune Evasion as Challenges to T B Vaccine Design. Cell Host Microbe 24:34-42.
4. Lyadova I, Nikitina I. 2019. Cell DifferentiationDegree as a Factor Determining the Role for Different T-Helper Populations in Tuberculosis Protection. Front Immunol 10:972.
5. Scriba T J, Kaufmann S H, Henri Lambert P, Sanicas M, Martin C, Neyrolles O. 2016. Vaccination Against Tuberculosis With Whole-Cell Mycobacterial Vaccines. J Infect Dis 214:659-64.
6. Cruz A, Fraga A G, Fountain J J, Rangel-Moreno J, Torrado E, Saraiva M, Pereira D R, Randall T D, Pedrosa J, Cooper A M, Castro A G. 2010. Pathological role of interleukin 17 in mice subjected to repeated BCG vaccination after infection with Mycobacterium tuberculosis. J Exp Med 207:1609-16.
7. Dalmia N, Ramsay A J. 2012. Prime-boost approaches to tuberculosis vaccine development. Expert Rev Vaccines 11:1221-33.
8. Groschel M I, Sayes F, Shin S J, Frigui W, Pawlik A, Orgeur M, Canetti R, Honore N, Simeone R, van der Werf T S, Bitter W, Cho S N, Majlessi L, Brosch R. 2017. Recombinant BCG Expressing ESX-1 of Mycobacterium marinum Combines Low Virulence with Cytosolic Immune Signaling and Improved T B Protection. Cell Rep 18:2752-2765.
9. Sayes F, Pawlik A, Frigui W, Groschel M I, Crommelynck S, Fayolle C, Cia F, Bancroft G J, Bottai D, Leclerc C, Brosch R, Majlessi L. 2016. CD4⁺ T Cells Recognizing P E/PPE Antigens Directly or via Cross Reactivity Are Protective against Pulmonary Mycobacterium tuberculosis Infection. PLOS Pathog 12: e1005770.
10. Arhel N J, Souquere-Besse S, Munier S, Souque P, Guadagnini S, Rutherford S, Prevost M C, Allen T D, Charneau P. 2007. HIV-1 DNA Flap formation promotes uncoating of the pre-integration complex at the nuclear pore. EMBO J 26:3025-37.
11. Di Nunzio F, Felix T, Arhel N J, Nisole S, Charneau P, Beignon A S. 2012. HIV-derived vectors for therapy and vaccination against HIV. Vaccine 30:2499-509.
12. Hu B, Tai A, Wang P. 2011. Immunization delivered by lentiviral vectors for cancer and infectious diseases. Immunol Rev 239:45-61.
13. Sirven A, Pflumio F, Zennou V, Titeux M, Vainchenker W, Coulombel L, Dubart-Kupperschmitt A, Charneau P. 2000. The human immunodeficiency virus type-1 central DNA flap is a crucial determinant for lentiviral vector nuclear import and gene transduction of human hematopoietic stem cells. Blood 96:4103-10.
14. Zennou V, Petit C, Guetard D, Nerhbass U, Montagnier L, Charneau P. 2000. HIV-1 genome nuclear import is mediated by a central DNA flap. Cell 101:173-85.
15. Zennou V, Serguera C, Sarkis C, Colin P, Perret E, Mallet J, Charneau P. 2001. The HIV-1 DNA flap stimulates HIV vector-mediated cell transduction in the brain. Nat Biotechnol 19:446-50.
16. Lindestam Arlehamn C S, Gerasimova A, Mele F, Henderson R, Swann J, Greenbaum J A, Kim Y, Sidney J, James E A, Taplitz R, Mckinney D M, Kwok W W, Grey H, Sallusto F, Peters B, Sette A. 2013. Memory T cells in latent Mycobacterium tuberculosis infection are directed against three antigenic islands and largely contained in a CXCR3+CCR6+ Th1 subset. PLOS Pathog 9: e1003130.
17. Majlessi L, Prados-Rosales R, Casadevall A, Brosch R. 2015. Release of mycobacterial antigens. Immunol Rev 264:25-45.
18. Cole S T, Brosch R, Parkhill J, Garnier T, Churcher C, Harris D, Gordon S V, Eiglmeier K, Gas S, Barry C E, 3rd, Tekaia F, Badcock K, Basham D, Brown D, Chillingworth T, Connor R, Davies R, Devlin K, Feltwell T, Gentles S, Hamlin N, Holroyd S, Hornsby T, Jagels K, Krogh A, McLean J, Moule S, Murphy L, Oliver K, Osborne J, Quail M A, Rajandream M A, Rogers J, Rutter S, Seeger K, Skelton J, Squares R, Squares S, Sulston J E, Taylor K, Whitehead S, Barrell B G. 1998. Deciphering the biology of Mycobacterium tuberculosis from the complete genome sequence. Nature 393:537-44.
19. Banu S, Honore N, Saint-Joanis B, Philpott D, Prevost M C, Cole S T. 2002. Are the PE-PGRS proteins of Mycobacterium tuberculosis variable surface antigens? Mol Microbiol 44:9-19.
20. Iantomasi R, Sali M, Cascioferro A, Palucci I, Zumbo A, Soldini S, Rocca S, Greco E, Maulucci G, De Spirito M, Fraziano M, Fadda G, Manganelli R, Delogu G. 2011. PE_PGRS30 is required for the full virulence of Mycobacterium tuberculosis. Cell Microbiol doi: 10.1111/j. 1462-5822.2011.01721.x: doi: 10.1111/j. 1462-5822.
21. Abdallah A M, Verboom T, Weerdenburg E M, Gey van Pittius N C, Mahasha P W, Jimenez C, Parra M, Cadieux N, Brennan M J, Appelmelk B, Bitter W. 2009. PPE and PE_PGRS proteins of Mycobacterium marinum are transported via the type VII secretion system ESX-5. Molecular Microbiology 73:329-340.
22. Groschel M I, Sayes F, Simeone R, Majlessi L, Brosch R. 2016. ESX secretion systems: mycobacterial evolution to counter host immunity. Nat Rev Microbiol 14:677-691.
23. Sayes F, Blanc C, Ates L S, Deboosere N, Orgeur M, Le Chevalier F, Groschel M I, Frigui W, Song O R, Lo-Man R, Brossier F, Sougakoff W, Bottai D, Brodin P, Charneau P, Brosch R, Majlessi L. 2018. Multiplexed Quantitation of Intraphagocyte Mycobacterium tuberculosis Secreted Protein Effectors. Cell Rep 23:1072-1084.
24. Hervas-Stubbs S, Majlessi L, Simsova M, Morova J, Rojas M J, Nouze C, Brodin P, Sebo P, Leclerc C. 2006. High frequency of CD4⁺ T cells specific for the TB10.4 protein correlates with protection against Mycobacterium tuberculosis infection. Infect Immun 74:3396-407.
25. Majlessi L, Rojas M J, Brodin P, Leclerc C. 2003. CD8⁺-T-cell responses of Mycobacterium-infected mice to a newly identified major histocompatibility complex class I-restricted epitope shared by proteins of the ESAT-6 family. Infect Immun 71:7173-7.
26. Diebold S S, Cotten M, Koch N, Zenke M. 2001. MHC class II presentation of endogenously expressed antigens by transfected dendritic cells. Gene Ther 8:487-93. Rowe H M, Lopes L, Ikeda Y, Bailey R, Barde I, Zenke M, Chain B M, Collins M K. 2006. 27. Immunization with a lentiviral vector stimulates both CD4 and CD8 T cell responses to an ovalbumin transgene. Mol Ther 13:310-9.
28. Kreiter S, Selmi A, Diken M, Sebastian M, Osterloh P, Schild H, Huber C, Tureci O, Sahin U. 2008. Increased antigen presentation efficiency by coupling antigens to MHC class | trafficking signals. J Immunol 180:309-18.
29. Liechtenstein T, Dufait I, Bricogne C, Lanna A, Pen J, Breckpot K, Escors D. 2012. P D-L1/PD-1 Co-Stimulation, a Brake for T cell Activation and a T cell Differentiation Signal. J Clin Cell Immunol S12.
30. Cousin C, Oberkampf M, Felix T, Rosenbaum P, Weil R, Fabrega S, Morante V, Negri D, Cara A, Dadaglio G, Leclerc C. 2019. Persistence of Integrase-Deficient Lentiviral Vectors Correlates with the Induction of STING-Independent CD8 (+) T Cell Responses. Cell Rep 26:1242-1257 e7.
31. Pichlmair A, Diebold S S, Gschmeissner S, Takeuchi Y, Ikeda Y, Collins M K, Reis e Sousa C. 2007. Tubulovesicular structures within vesicular stomatitis virus G protein-pseudotyped lentiviral vector preparations carry DNA and stimulate antiviral responses via Toll-like receptor 9. J Virol 81:539-47.
32. Brown B D, Sitia G, Annoni A, Hauben E, Sergi L S, Zingale A, Roncarolo M G, Guidotti L G, Naldini L. 2007. In vivo administration of lentiviral vectors triggers a type I interferon response that restricts hepatocyte gene transfer and promotes vector clearance. Blood 109:2797-805.
33. Van Dis E, Sogi K M, Rae C S, Sivick K E, Surh N H, Leong M L, Kanne D B, Metchette K, Leong J J, Bruml J R, Chen V, Heydari K, Cadieux N, Evans T, McWhirter S M, Dubensky T W, Jr., Portnoy D A, Stanley S A. 2018. STING-Activating Adjuvants Elicit a Th17 Immune Response and Protect against Mycobacterium tuberculosis Infection. Cell Rep 23:1435-1447.
34. Anderson K G, Mayer-Barber K, Sung H, Beura L, James B R, Taylor J J, Qunaj L, Griffith T S, Vezys V, Barber D L, Masopust D. 2014. Intravascular staining for discrimination of vascular and tissue leukocytes. Nat Protoc 9:209-22.
35. Vroman H, Hendriks R W, Kool M. 2017. Dendritic Cell Subsets in Asthma: Impaired Tolerance or Exaggerated Inflammation? Front Immunol 8:941.
36. Gibbings S L, Thomas S M, Atif S M, McCubbrey A L, Desch A N, Danhorn T, Leach S M, Bratton D L, Henson P M, Janssen W J, Jakubzick C V. 2017. Three Unique Interstitial Macrophages in the Murine Lung at Steady State. Am J Respir Cell Mol Biol 57:66-76.
37. Kubo M. 2018. Mast cells and basophils in allergic inflammation. Curr Opin Immunol 54:74-79.
38. Lyadova I V. 2017. Neutrophils in Tuberculosis: Heterogeneity Shapes the Way? Mediators Inflamm 2017:8619307.
39. Schenkel J M, Masopust D. 2014. Tissue-resident memory T cells. Immunity 41:886-97.
40. Turner D L, Bickham K L, Thome J J, Kim C Y, D'Ovidio F, Wherry E J, Farber D L. 2014. Lung niches for the generation and maintenance of tissue-resident memory T cells. Mucosal Immunol 7:501-10.
41. D'Souza S, Rosseels V, Romano M, Tanghe A, Denis O, Jurion F, Castiglione N, Vanonckelen A, Palfliet K, Huygen K. 2003. Mapping of murine Th1 helper T-Cell epitopes of mycolyl transferases Ag85A, Ag85B, and Ag85C from Mycobacterium tuberculosis. Infect Immun 71:483-93.
42. Sayes F, Sun L, Di Luca M, Simeone R, Degaiffier N, Fiette L, Esin S, Brosch R, Bottai D, Leclerc C, Majlessi L. 2012. Strong immunogenicity and cross-reactivity of Mycobacterium tuberculosis ESX-5 type VII secretion: encoded PE-PPE proteins predicts vaccine potential. Cell Host Microbe 11:352-63.
43. Majlessi L, Brodin P, Brosch R, Rojas M J, Khun H, Huerre M, Cole S T, Leclerc C. 2005. Influence of ESAT-6 secretion system 1 (RD1) of Mycobacterium tuberculosis on the interaction between mycobacteria and the host immune system. J Immunol 174:3570-9.
44. Pym A S, Brodin P, Majlessi L, Brosch R, Demangel C, Williams A, Griffiths K E, Marchal 44. G, Leclerc C, Cole S T. 2003. Recombinant BCG exporting ESAT-6 confers enhanced protection against tuberculosis. Nat Med 9:533-9.
45. Haas C T, Roe J K, Pollara G, Mehta M, Noursadeghi M. 2016. Diagnostic ‘omics’ for active tuberculosis. BMC Med 14:37.
46. Bhatt K, Verma S, Ellner J J, Salgame P. 2015. Quest for correlates of protection 46. against tuberculosis. Clin Vaccine Immunol 22:258-66.
47. Tameris M D, Hatherill M, Landry B S, Scriba T J, Snowden M A, Lockhart S, Shea J E, McClain J B, Hussey G D, Hanekom W A, Mahomed H, McShane H, Team MATS. 2013. Safety and efficacy of MVA85A, a new tuberculosis vaccine, in infants previously vaccinated with BCG: a randomised, placebo-controlled phase 2b trial. Lancet 381:1021-8.
48. Britton G, MacDonald D C, Brown J S, Collins M K, Goodman A L. 2015. Using a prime and pull approach, lentivector vaccines expressing Ag85A induce immunogenicity but fail to induce protection against Mycobacterium bovis bacillus Calmette-Guerin challenge in mice. Immunology 146:264-70.
49. Xu Y, Yang E, Wang J, Li R, Li G, Liu G, Song N, Huang Q, Kong C, Wang H. 2014. Prime-boost bacillus Calmette-Guerin vaccination with lentivirus-vectored and DNA-based vaccines expressing antigens Ag85B and Rv3425 improves protective efficacy against Mycobacterium tuberculosis in mice. Immunology 143:277-86.
50. Bottai D, Brosch R. 2009. Mycobacterial P E, PPE and ESX clusters: novel insights into the secretion of these most unusual protein families. Mol Microbiol 73:325-8.
51. Brennan M J, Delogu G. 2002. The P E multigene family: a ‘molecular mantra’ for mycobacteria. Trends Microbiol 10:246-9.
52. Voskuil M I, Schnappinger D, Rutherford R, Liu Y, Schoolnik G K. 2004. Regulation of the Mycobacterium tuberculosis P E/PPE genes. Tuberculosis (Edinb) 84:256-62.
53. Bottai D, Di Luca M, Majlessi L, Frigui W, Simeone R, Sayes F, Bitter W, Brennan M J, Leclerc C, Batoni G, Campa M, Brosch R, Esin S. 2012. Disruption of the ESX-5 system of Mycobacterium tuberculosis causes loss of PPE protein secretion, reduction of cell wall integrity and strong attenuation. Mol Microbiol 83:1195-209.
54. Fishbein S, van Wyk N, Warren R M, Sampson S L. 2015. Phylogeny to function: P E/PPE protein evolution and impact on Mycobacterium tuberculosis pathogenicity. Mol Microbiol 96:901-16.
55. Gey van Pittius N C, Sampson S L, Lee H, Kim Y, van Helden P D, Warren R M. 2006. Evolution and expansion of the Mycobacterium tuberculosis P E and PPE multigene families and their association with the duplication of the ESAT-6 (esx) gene cluster regions. BMC Evol Biol 6:95.
56. Ku M W, Bourgine M, Authie P, Lopez J, Nemirov N, Moncoq F, Noirat A, Vesin B, Nevo F, Blanc C, Souque P, Simon E, Tabbal H, Mouquet H, Anna F, Martin A, Escriou, Majlessi L, Charneau P. 2020. Intranasal Immunization with a Lentiviral Vector Coding for SARS-COV-2 Spike Protein Confers Vigorous Protection in Pre-Clinical Animal Models. BioRxiv
57. Chiu C, Openshaw P J. 2015. Antiviral B cell and T cell immunity in the lungs. Nat Immunol 16:18-26.
58. Holmgren J, Czerkinsky C. 2005. Mucosal immunity and vaccines. Nat Med 11: S45-53.
59. Mueller S N, Mackay L K. 2016. Tissue-resident memory T cells: local specialists in immune defence. Nat Rev Immunol 16:79-89.
60. Park C O, Kupper T S. 2015. The emerging role of resident memory T cells in protective immunity and inflammatory disease. Nat Med 21:688-97.
61. Dong H, Stanek O, Salvador F R, Langer U, Morillon E, Ung C, Sebo P, Leclerc C, Majlessi L. 2013. Induction of protective immunity against Mycobacterium tuberculosis by delivery of ESX antigens into airway dendritic cells. Mucosal Immunol 6:522-34.
62. Bull N C, Stylianou E, Kaveh D A, Pinpathomrat N, Pasricha J, Harrington-Kandt R, Garcia-Pelayo M C, Hogarth P J, McShane H. 2019. Enhanced protection conferred by mucosal BCG vaccination associates with presence of antigen-specific lung tissue-resident PD-1 (+) KLRG1 (−) CD4 (+) T cells. Mucosal Immunol 12:555-564.
63. Florido M, Muflihah H, Lin LCW, Xia Y, Sierro F, Palendira M, Feng C G, Bertolino P, Stambas J, Triccas J A, Britton W J. 2018. Pulmonary immunization with a recombinant influenza A virus vaccine induces lung-resident CD4 (+) memory T cells that are associated with protection against tuberculosis. Mucosal Immunol 11:1743-1752.
64. Perdomo C, Zedler U, Kuhl A A, Lozza L, Saikali P, Sander L E, Vogelzang A, Kaufmann S H, Kupz A. 2016. Mucosal BCG Vaccination Induces Protective Lung-Resident Memory T Cell Populations against Tuberculosis. mBio 7.
65. Sakai S, Kauffman K D, Schenkel J M, McBerry C C, Mayer-Barber K D, Masopust D, Barber D L. 2014. Cutting edge: control of Mycobacterium tuberculosis infection by a subset of lung parenchyma-homing CD4 T cells. J Immunol 192:2965-9.
66. Desel C, Dorhoi A, Bandermann S, Grode L, Eisele B, Kaufmann S H. 2011. Recombinant BCG DeltaureC hly+ induces superior protection over parental BCG by stimulating a balanced combination of type 1 and type 17 cytokine responses. J Infect Dis 204:1573-84.
67. Shen H, Chen Z W. 2018. The crucial roles of Th17-related cytokines/signal pathways in M. tuberculosis infection. Cell Mol Immunol 15:216-225.
68. Raeven RHM, Rockx-Brouwer D, Kanojia G, van der Maas L, Bindels THE, Ten Have R, van Riet E, Metz B, Kersten GFA. 2020. Intranasal immunization with outer membrane vesicle pertussis vaccine confers broad protection through mucosal IgA and Th17 responses. Sci Rep 10:7396.
69. Kim T S, Gorski S A, Hahn S, Murphy K M, Braciale T J. 2014. Distinct dendritic cell subsets dictate the fate decision between effector and memory CD8 (+) T cell differentiation by a CD24-dependent mechanism. Immunity 40:400-13.
70. Ng S L, Teo Y J, Setiagani Y A, Karjalainen K, Ruedl C. 2018. Type 1 Conventional CD103 (+) Dendritic Cells Control Effector CD8 (+) T Cell Migration, Survival, and Memory Responses During Influenza Infection. Front Immunol 9:3043.
71. Zelante T, Wong A Y, Ping T J, Chen J, Sumatoh H R, Vigano E, Hong Bing Y, Lee B, Zolezzi F, Fric J, Newell E W, Mortellaro A, Poidinger M, Puccetti P, Ricciardi-Castagnoli P. 2015. CD103 (+) Dendritic Cells Control Th17 Cell Function in the Lung. Cell Rep 12:1789-801.
72. Kaufmann E, Sanz J, Dunn J L, Khan N, Mendonca L E, Pacis A, Tzelepis F, Pernet E, Dumaine A, Grenier J C, Mailhot-Leonard F, Ahmed E, Belle J, Besla R, Mazer B, King I L, Nijnik A, Robbins C S, Barreiro L B, Divangahi M. 2018. BCG Educates Hematopoietic Stem Cells to Generate Protective Innate Immunity against Tuberculosis. Cell 172:176-190 e19.
73. Kaufmann S H. 2010. Future vaccination strategies against tuberculosis: thinking outside the box. Immunity 33:567-77.
74. Jones G W, Hill D G, Jones S A. 2016. Understanding Immune Cells in Tertiary Lymphoid Organ Development: It Is All Starting to Come Together. Front Immunol 7:401.
75. Allix-Beguec C, Harmsen D, Weniger T, Supply P, Niemann S. 2008. Evaluation and strategy for use of MIRU-VNTRplus, a multifunctional database for online analysis of genotyping data and phylogenetic identification of Mycobacterium tuberculosis complex isolates. J Clin Microbiol 46:2692-9.
76. Caton M L, Smith-Raska M R, Reizis B. 2007. Notch-RBP-J signaling controls the homeostasis of CD8-dendritic cells in the spleen. J Exp Med 204:1653-64.
77. Le Bon A, Thompson C, Kamphuis E, Durand V, Rossmann C, Kalinke U, Tough D F. 2006. Cutting edge: enhancement of antibody responses through direct stimulation of B and T cells by type | IFN. J Immunol 176:2074-8.

1. A recombinant lentiviral vector genome comprising a polynucleotide encoding a fusion polypeptide, wherein said fusion polypeptide comprises, arranged from N-terminal to C-terminal ends:
a first polypeptide comprising (i) an MHC-II-associated light invariant chain (li), preferably of SEQ ID No. 11, or (ii) the transmembrane domain of the transferrin receptor (TfR), preferably of SEQ ID No. 13, and
at least one antigenic polypeptide of a pathogen.

2. The recombinant lentiviral vector genome according to claim 1, wherein said antigenic polypeptide is a mono-antigenic polypeptide comprising one antigen of a pathogen or immunogenic fragment thereof, or is a poly-antigenic polypeptide comprising at least two antigens of one or more pathogens or immunogenic fragments thereof.

3. The recombinant lentiviral vector genome according to claim 1, wherein the pathogen is a bacterial, parasite or viral pathogen, in particular a pathogen associated with an acute or chronic respiratory infectious disease in a mammal, more particularly is Mycobacterium tuberculosis, an influenza virus or a coronavirus such as SARS-CoV-2.

4. The recombinant lentiviral vector genome according to claim 3, wherein said antigenic polypeptide comprises one or more Mycobacterium tuberculosis (Mtb) antigens selected from EsxA, EspC, EsxH, PE19 or Ag85A, or an immunogenic fragment thereof, in particular one of the following Mtb antigenic combinations:
(a) EsxH;
(b) EsxH and EsxA;
(c) EsxH, EsxA and PE19;
(d) EsxH, EsxA, EspC and PE19;
(e) EsxH, EsxA, EspC, PE19 and Ag85A;
or immunogenic fragments thereof.

5. The recombinant lentiviral vector genome according to claim 1, wherein said genome is obtained from a pFLAP vector plasmid, in particular the vector plasmid of nucleotide sequence SEQ ID No. 20, wherein the polynucleotide encoding the fusion polypeptide has been cloned under control of a promoter functional in mammalian cells, in particular the CMV promoter, the human beta-2 microglobulin promoter, the SP1-human beta-2 microglobulin promoter of SEQ ID No. 21 or the composite BCUAG promoter of SEQ ID No. 22 and wherein the vector optionally comprises post-transcriptional regulatory element of the woodchuck hepatitis virus (WPRE), in particular a mutant WPRE as set forth in SEQ ID No. 23.

6. A DNA plasmid comprising the recombinant lentiviral vector genome according to claim 1, in particular wherein said genome is inserted within a pFLAP vector plasmid, preferably the vector plasmid of nucleotide sequence SEQ ID No. 20, wherein the fusion polypeptide encoded by the polynucleotide comprised within the recombinant lentiviral vector genome is inserted between restriction sites BamHI and Xhol in replacement of the GFP sequence.

7. A recombinant lentiviral vector particle which comprises the recombinant lentiviral vector genome according to claim 1.

8. The recombinant lentiviral vector particle according to claim 7, which is a recombinant integration-deficient lentiviral vector particle, in particular wherein the recombinant integration-deficient lentiviral vector particle is a HIV-1 based vector particle and is integrase deficient as a result of a mutation of the integrase gene encoded in the genome of the lentivirus in such a way that the integrase is not expressed or not functionally expressed, in particular the mutation in the integrase gene leads to the expression of an integrase substituted on its amino acid residue 64, in particular the substitution is D64V in the catalytic domain of the HIV-1 integrase encoded by Pol.

9. The recombinant lentiviral vector particle according to claim 7, wherein said recombinant lentiviral vector particle is a recombinant replication-incompetent pseudotyped lentiviral vector particle, in particular a replication-incompetent pseudotyped HIV-1 lentiviral vector particle, in particular wherein the lentiviral vector particle is pseudotyped with the glycoprotein G from a Vesicular Stomatitis Virus (V-SVG) of Indiana or of New-Jersey serotype.

10. A host cell, preferably a mammalian host cell, transfected with a DNA plasmid according to claim 6, in particular wherein said host cell is a HEK-293T cell line or a K562 cell line.

11. A pharmaceutical composition, in particular a vaccine composition, suitable for administration to a mammalian host, comprising a recombinant lentiviral vector particle of claim 7 together with one or more pharmaceutically acceptable excipient(s) suitable for administration to a host in need thereof, in particular a human host.

12. The pharmaceutical composition of claim 11, further comprising an adjuvant, in particular a pro-Th1 and/or pro-Th17 adjuvant such as polyinosinic-polycytidylic acid (polyI:C) or a derivative thereof, or a cyclic dinucleotide adjuvant, in particular cyclic Guanine-Adenine dinucleotide (cGAMP).

13. The pharmaceutical composition of claim 11, for use in the elicitation of a protective, preferentially prophylactic, immune response by the elicitation of antibodies directed against the antigenic polypeptide or immunogenic fragments thereof in a host in need thereof, in particular a human host.

14. The pharmaceutical composition of claim 13, wherein the immune response involves the induction of MHC-I restricted presentation and MHC-II restricted presentation of the antigenic polypeptide or immunogenic fragments thereof, by an antigen-presenting cell, in particular a dendritic cell, and the induction of a CD4- and CD8-mediated cellular immune response.

15. The pharmaceutical composition of claim 11, for use in preventing and/or treating an infection by a pathogen in a mammalian host in need thereof, in particular a human host in particular an infection by a pathogen associated with an acute or chronic respiratory infectious disease in a mammal.

16. A method for the preparation of recombinant lentiviral vector particles suitable for the preparation of a pharmaceutical composition, in particular a vaccine composition, comprising the following steps:
a) transfecting the recombinant lentiviral transfer vector carrying the lentiviral vector genome comprising a polynucleotide encoding a fusion polypeptide, according to claim 1 in a host cell, for example a HEK-293T cell line or a K562 cell line;
b) co-transfecting the cell of step a) with: (i) a plasmid vector encoding envelope proteins and with a plasmid vector encoding the lentiviral GAG and POL or mutated POL protein as packaging construct; and (ii) a plasmid encoding VSV-G Indiana or New Jersey envelope,
c) culturing the host cell under conditions suitable for the production of recombinant lentiviral vector particles expressing the fusion polypeptide;
d) recovering the recombinant lentiviral particles expressing the fusion polypeptide.
